(19) United States
(12) Patent Application Publication
(10) Pub. No.: US 2024/0317740 A1
(43) Pub. Date: Sep. 26, 2024

(54) NOVEL COMPOUNDS AND THEIR USE

(71) Applicant: Debiopharm International S.A., Lausanne (CH)

(72) Inventors: Vincent Gerusz, Neuvecelle (FR); Terry Finn, Geneve (CH); Heinz Pauls, Guelph (CA); Judd Berman, Encinitas, CA (US); Juan Bravo, Cudrefin (CH)

(73) Assignee: Debiopharm International S.A., Lausanne (CH)

(21) Appl. No.: 18/572,589

(22) PCT Filed: Jun. 22, 2022
(86) PCT No.: PCT/EP2022/067039

§ 371 Date: Dec. 20, 2023

(30) Foreign Application Priority Data
Jun. 23, 2021 (EP) EP 21 181 117.9

(51) Int. Cl. C07D 471/04; A61K 31/55; A61K 31/551

(52) U.S. Cl. CPC C07D 471/04; A61K 31/55; A61K 31/551

(57) ABSTRACT

The present invention provides compounds of the general formula (I) or a pharmaceutically acceptable prodrugs, salts and/or solvates thereof, wherein LHS is formula (II). And wherein, the asterisk (*) marks the point of attachment; These compounds exhibit antibacterial activity against Gram-negative and Gram-positive bacteria, especially S. aureus, E. coli, K. pneumoniae and A. baumannii. Pharmaceutical compositions containing these compounds, therapeutic uses thereof and methods for manufacturing the same are also provided.

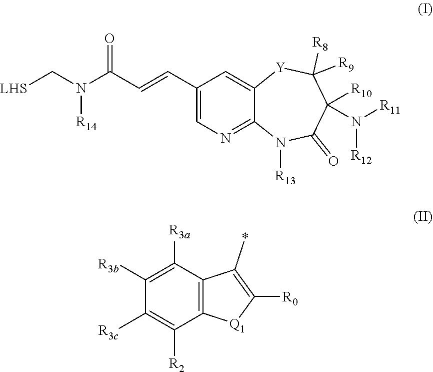

### FIELD OF THE INVENTION

The present invention relates to antibiotic compounds, pharmaceutical compositions comprising them, and to the use of these compounds and compositions for the treatment of bacterial infections. The invention further relates to methods of making said compounds of the invention.

### BACKGROUND OF THE INVENTION

Antibiotic resistance is rising to dangerously high levels in all parts of the world, threatening our ability to effectively treat and prevent an ever-increasing range of infections. Accordingly, there is a need for the development of novel antibiotic compounds that may show activity in cases where established antibiotics fail.

Whilst all types of bacteria (both Gram-negative and Gram-positive) are believed to have developed some measure of antibiotic resistance, certain bacterial species are more associated with antibiotic resistance than others e.g. Staphylococcus aureus (S. aureus), Klebsiella pneumoniae (K. pneumoniae), Acinetobacter baumannii (A. baumannii) and Escherichia coli (E. coli). Accordingly, there may be a particular need for novel antibiotic compound active against one or more of these species of bacteria.

A recently developed new class of antibiotics compounds are Fabl inhibitors. These compounds inhibit the NADH-dependent enoyl reductase (Fabl) from the type II bacterial fatty acid biosynthesis pathway (FAS-II), thereby providing an alternative approach for treating bacterial infections in cases where established antibiotics fail. Advantageously, this Fabl mode of action is not expected to display any cross resistance to established antibiotics. However, whilst known Fabl inhibitor compounds can be extremely effective against some bacterial species, said compounds may not be active or may have inadequate activity against other species such as S. aureus, E. coli, A. baumannii, and K. pneumoniae, and in particular the Gram-negative bacterial species E. coli, A. baumannii, and K. pneumoniae. For instance, WO 2020/099341 A1 discloses Fabl inhibitors that are effective in the treatment of N. gonorrhoeae bacterial infections. It remains uncertain whether these compounds may also be used for treating infections by E. coli, A. baumannii, and K. pneumoniae. This may be because of the challenge of penetrating both the outer and inner membranes of these Gram-negative bacteria, a challenge that can be further compounded by efflux. Accordingly, there is still a need for compounds and pharmaceutical compositions comprising the same that may show antibiotic activity (especially in cases where established antibiotics fail) against Gram-positive and/or Gram-negative bacteria, and especially against one or more of S. aureus, E. coli, K. pneumoniae and A. baumannii, and most especially E. coli, K. pneumoniae and A. baumannii. Furthermore, it is preferable that such compounds show favourable lung exposure and do not give rise to cross resistance to established antibiotics, and it is desirable that such compounds give rise to a low/acceptable rate of side effects.

It is an object of the invention to address one or more of these aforementioned needs. Further objectives and problems underlying the present invention may become apparent from the subsequent description of the invention.

### SUMMARY OF THE INVENTION

Surprisingly the inventors have found that an objective of the invention may be accomplished by the compounds, pharmaceutical compositions, therapeutic uses thereof, and synthetic methods of the present invention. The present invention includes compounds, pharmaceutically acceptable salts, prodrugs, and/or solvates as specified in the appended claim 1. Preferred embodiments are specified in the subsequent claims 2 to 12.

The invention further provides such compounds for use in medicine. Pharmaceutical compositions comprising the compounds of the present invention are also provided. These are specified in appended claim 13.

Yet another aspect of the present invention relates to the provision of the compounds described herein for use in a method of therapy wherein preferably the method of therapy is a method of treating a bacterial infection and wherein preferably the bacterial infection is associated with one or more of bacteria selected from the group consisting of: S. aureus, E. coli, Klebsiella pneumoniae and A. baumannii and most preferably wherein the bacterial infection is associated with A. baumannii and is preferably pneumonia and most preferably nosocomial pneumonia. The present invention thus also provides methods for treating a bacterial infection in a patient in need thereof, said method comprising the administration of a compound described herein to the patient. Preferably said bacterial infection is associated with one or more of bacteria selected from the group consisting of: S. aureus, E. coli, Klebsiella pneumoniae and A. baumannii and most preferably wherein the bacterial infection is associated with A. baumannii. The infection is preferably pneumonia and most preferably nosocomial pneumonia.

The present invention further provides methods for manufacturing the compounds of the present invention, for instance as specified in appended claim 15.

### DETAILED DESCRIPTION OF THE INVENTION

### Definitions

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

In some embodiments, the term “about” refers to a deviation of ±10% from the recited value. When the word “about” is used herein in reference to a number, it should be understood that still another embodiment of the invention includes that number not modified by the presence of the word “about” “Administering” or “administration of” a drug to a patient (and grammatical equivalents of this phrase) refers to direct administration, which may be administration to a patient by a medical professional or may be self-administration, and/or indirect administration, which may be the act of prescribing a drug.

E.g., a physician who instructs a patient to self-administer a drug or provides a patient with a prescription for a drug is administering the drug to the patient.

“Dose” and “dosage” refer to a specific amount of active or therapeutic agents for administration. Such amounts are included in a “dosage form,” which refers to physically discrete units suitable as unitary dosages for human subjects and other mammals, each unit containing a predetermined quantity of active agent calculated to produce the desired onset, tolerability, and therapeutic effects, in association with one or more suitable pharmaceutical excipients such as carriers.

The terms “treatment” and “therapy”, as used in the present application, refer to a set of hygienic, pharmacological, surgical and/or physical means used with the intent to cure and/or alleviate a disease and/or symptoms with the goal of remediating the health problem. The terms “treatment” and “therapy” include preventive and curative methods, since both are directed to the maintenance and/or reestablishment of the health of an individual or animal. Regardless of the origin of the symptoms, disease and disability, the administration of a suitable medicament to alleviate and/or cure a health problem should be interpreted as a form of treatment or therapy within the context of this application.

“Unit dosage form” as used herein refers to a physically discrete unit of therapeutic formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

The articles “a” and “an” are used herein to refer to one or to more than one (i.e., to at least one) of the grammatical object of the article. By way of example, “an element” means one element or more than one element.

The term “including” is used to mean “including but not limited to”. “Including” and “including but not limited to” are used interchangeably. The term “comprising” is used to have the same meaning as “including”. The term “consisting of” is used to indicate that the listed element(s) is/are present but no other unmentioned elements. The term “comprising” is used to include the meaning of “consisting of” as a preferred embodiment.

The term “Fabl” is art-recognized and refers to the bacterial enzyme believed to function as an enoyl-acyl carrier protein (ACP) reductase in the final step of the four reactions involved in each cycle of bacterial fatty acid biosynthesis. This enzyme is believed to be widely distributed in bacteria and plants.

The term “enzyme inhibitor” refers to any compound that prevents an enzyme from effectively carrying out its respective biochemical roles. Therefore a “Fabl inhibitor” is any compound that inhibits Fabl from carrying out its biochemical role. The amount of inhibition of the enzyme by any such compound will vary and is described herein and elsewhere.

The term “antibiotic agent” or “antibacterial agent” shall mean any drug that is useful in treating, preventing, or otherwise reducing the severity of any bacterial disorder, or any complications thereof, including any of the conditions, disease, or complications arising therefrom and/or described herein.

Antibiotic agents include, for example, cephalosporins, quinolones and fluoroquinolones, penicillins and beta lactamase inhibitors, carbapenems, monobactams, macrolides and lincosamides, glycopeptides, rifampin, oxazolidinones, tetracyclines, aminoglycosides, streptogramins, sulfonamides, and the like.

Other antibiotic or antibacterial agents are disclosed herein, and are known to those of skill in the art. In certain embodiments, the term “antibiotic agent” does not include an agent that is a Fabl inhibitor, so that the combinations of the present invention in certain instances will include one agent that is a Fabl inhibitor and another agent that is not.

The term “drug” as used herein refers to any substance falling within at least one of the definitions given in Article 1, Items 2(a), 2(b) or 3a. of Directive 2001/83/EC of Nov. 6, 2001 in the version of Nov. 16, 2012 or in Article 1, Items 2(a) or 2(b) of Directive 2001/82/EC of Nov. 6, 2001 in the version of Aug. 7, 2009 and in Article 2 of Regulation (EC) No. 726/2004 of Mar. 31, 2004.

The term “illness” as used herein refers to any illness caused by or related to infection by an organism.

The term “bacterial illness” as used herein refers to any illness caused by or related to infection by bacteria.

The term “cis” is art-recognized and refers to the arrangement of two atoms or groups around a double bond such that the atoms or groups are on the same side of the double bond. Cis configurations are often labeled as (Z) configurations.

The term “trans” is art-recognized and refers to the arrangement of two atoms or groups around a double bond such that the atoms or groups are on the opposite sides of a double bond. Trans configurations are often labeled as (E) configurations.

The term “therapeutic effect” is art-recognized and refers to a local or systemic effect in animals, particularly mammals, and more particularly humans caused by a pharmacologically active substance.

The term thus means any measurable effect in the diagnosis, cure, mitigation, treatment or prevention of disease or in the enhancement of desirable physical or mental development and/or conditions in an animal or human. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions of the present invention may be administered in a sufficient amount to produce a at a reasonable benefit/risk ratio applicable to such treatment.

The term “chiral” is art-recognized and refers to molecules which have the property of non-superimposability of the mirror image partner, while the term “achiral” refers to molecules which are superimposable on their mirror image partner. A “prochiral molecule” is a molecule which has the potential to be converted to a chiral molecule in a particular process.

The compounds of the disclosure may contain one or more chiral centers and/or double bonds and, therefore, exist as geometric isomers, enantiomers or diastereomers. The enantiomer and diastereomers may be designated by the symbols “(+)”, “(−)”, “R” or “S,” depending on the configuration of substituents around the stereogenic carbon atom, but the skilled artisan will recognize that a structure may denote one or more chiral centers implicitly. Mixtures of enantiomers or diastereomers may be designated “(±)” in nomenclature, but the skilled artisan will recognize that a structure may denote a chiral center implicitly. Geometric isomers, resulting from the arrangement of substituents around a carbon-carbon double bond or arrangement of substituents around a cycloalkyl or heterocyclic ring, can also exist in the compounds of the present invention.

The symbol-denotes a bond that may be a single, double or triple bond as described herein.

Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans” or “Z/E.”

The term “stereoisomers” when used herein consist of all geometric isomers, enantiomers or diastereomers. The present invention encompasses various stereoisomers of these compounds and mixtures thereof. Conformational isomers and rotamers of disclosed compounds are also contemplated.

The term “IC50” is art-recognised and refers to the effectiveness of a substance in inhibiting a given biological or biochemical process (or component of a process, i.e. an enzyme, cell, cell receptor or microorganism). IC50 represents the concentration of a drug e.g. a compound of the invention, that is required for 50% inhibition in vitro.

The term “MIC” is art-recognised and refers to the Minimum Inhibitory Concentration, that is the lowest concentration of an antimicrobial that will inhibit the visible growth of a microorganism following overnight incubation, usually reported as mg/L or μg/mL.

The term “antimicrobial” is art-recognized and refers to the ability of the compounds disclosed herein to prevent, inhibit or destroy the growth of microbes such as bacteria, fungi, protozoa and viruses.

The term “antibacterial” is art-recognized and refers to the ability of the compounds disclosed herein to prevent, inhibit or destroy the growth of microbes of bacteria.

The term “microbe” is art-recognized and refers to a microscopic organism. In certain embodiments the term microbe is applied to bacteria. In other embodiments the term refers to pathogenic forms of a microscopic organism.

Unless specified otherwise, the term “lung exposure” characterizes the amount of drug delivered to the lung of the patient. This property can be quantified by murine PK with the compound of interest being administered IV at 1 mg/kg and then determining lung AUC last. A “favorable lung exposure” may for example be considered to be lung exposure having a lung AUC last >1000 h*ng/ml. AUC values above this threshold value are considered to be particularly well suited for the treatment of pulmonary infections.

The term “alkyl” as used herein refers to a saturated straight or branched hydrocarbon, such as a straight or branched group of 1-8 or 1-6 carbon atoms referred to herein as C₁-C₈alkyl, or C₁-C₆alkyl, respectively. The term “lower alkyl” as used herein specifically refers to a saturated straight or branched hydrocarbon, such as a straight or branched group of 1-4 or 1-3 carbon atoms, referred to herein as C₁-C₄alkyl, and C₁-C₃alkyl, respectively. Exemplary alkyl groups and lower alkyl grous include, but are not limited to, methyl, ethyl, propyl, isopropyl, 2-methyl-1-propyl, 2-methyl-2-propyl, 2-methyl-1-butyl, 3-methyl-1-butyl, 3-methyl-2-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl.

Moreover, the term “alkyl” (or “lower alkyl”) includes also divalent saturated straight or branched hydrocarbon groups, which are sometimes referred to as alkanediyl groups or alkylene groups. The term “alkyl” not only covers unsubstituted groups but also “substituted alkyls”, i.e. it should be understood as optionally carrying one or more substituents at one or more positions. That is, it refers also to alkyl moieties having one or more (e.g. two, three, four, five, six, etc.) substituents, each replacing a hydrogen on a carbon of the hydrocarbon backbone. Such substituents may include, for example, a hydroxyl, a carbonyl group (wherein the carbonyl group carries a hydrogen atom, an alkyl group or another group as defined in this paragraph, such as to yield a carboxyl, an alkoxycarbonyl, a formyl, or an acyl group), a thiocarbonyl-containing group (wherein the carbonyl group carries a hydrogen atom, an alkyl group or another group as defined in this paragraph, such as to yield a thioester, a thioacetate, or a thioformate), an alkoxyl, a phosphoryl, a phosphonate, a phosphinate, a phosphate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, a cycloalkyl, a heterocycle or an aromatic or heteroaromatic moiety. In all instances, wherein the above-mentioned groups have more than one valency, the further free valency can be saturated by a hydrogen atom, an alkyl group, a cycloalkyl group, a heterocyclic group, an aryl group or a heteroaryl group. It will further be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate, phosphinate and phosphate), sulfonyl (including sulfate, sulfonamido, sulfamoyl and sulfonate), and silyl groups, as well as ethers, alkylthios, carbonyls (including ketones, aldehydes, carboxylates, and esters), nitrile and isonitrile. For the avoidance of doubt, an alkyl group carrying another alkyl group should not be regarded as an alkyl group substituted with another alkyl group, but as a single branched alkyl group.

The term “alkylene” is art-recognized and refers to a group corresponding to the alkyl group defined above, but having two free valencies. The alkylene group is sometimes also referred to as alkanediyl group.

The term “alkenyl” is art-recognized and refers to a group corresponding to the alkyl group defined above, but carrying one or more carbon-carbon double bonds. Of course, the total number of double bonds is restricted by the number of carbon atoms in the alkenyl group and in order to allow for at least one double bond, the alkenyl group must have at least two carbon atoms. Except for this difference, the definitions and characterizations given for the alkyl group above apply equally to the alkenyl group.

The term “alkynyl” is art-recognized and refers to a group corresponding to the alkyl group defined above, but carrying one or more carbon-carbon triple bonds. Of course, the total number of double bonds is restricted by the number of carbon atoms in the alkenyl group and in order to allow for at least one triple bond, the alkynyl group must have at least two carbon atoms. Except for this difference, the definitions and characterizations given for the alkyl group above apply equally to the alkynyl group.

The term “aryl” is art-recognized and refers to 5- or 6-membered single-ring aromatic groups that can be pure aromatic carbocycles or may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “heteroaryl” or “heteroaromatics.” The aromatic ring may be unsubstituted or substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, phosphate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic as defined above, while there is no particular restriction regarding the fused further ring or rings, which may for instance be cycloalkyls, cycloalkenyls, cycloalkynyls, aryls and/or heterocyclyls.

The term “aralkyl” or “arylalkyl” is art-recognized and refers to an alkyl group substituted with an aryl group (e.g., an aromatic or heteroaromatic group).

The term “carbocycle” is art-recognized and refers to an aromatic or non-aromatic ring in which each atom of the ring is carbon.

The term “cycloalkyl” as used herein refers to a monocyclic saturated or partically unsatured alkyl or alkenyl group of for example 3-6, or 4-6 carbons, referred to herein, e.g., as “C₃₋₆cycloalkyl” or “C₄₋₆ cycloalkyl,” and derived from a cycloalkane. Exemplary cycloalkyl groups include, but are not limited to, cyclohexane, cyclohexene, cyclopentane, cyclobutane, cyclopropane or cyclopentene. Said cycloalkyl group may be unsubstituted or substituted at one or more positions with one or more substituents as described above.

The terms “halogen” as used herein refer to F, Cl, Br, or I. “Halide” designates the corresponding anion of the halogens.

The term “amino” as used herein refers to any group of the general structure —NRₐR_{b}, wherein, unless specified otherwise, Rₐ and R_{b} are independently selected from the group consisting of H, alkyl, cycloalkyl, alkenyl, alkynyl, aryl, heteroaryl, heterocyclic groups, as well as any other substituent group listed above with respect to the scope of substituted alkyl groups, with the exception of carbonyl groups, thiocarbonyl groups, imine groups, and substituent groups in which attachment to the remaining molecule is via a heteroatom selected from N, O, S and P. Alternatively, Rₐ and R_{b} may represent hydrocarbon groups that are linked to form a heterocycle together with the nitrogen atom to which they are attached.

The term “heteroaryl” as used herein refers to a monocyclic aromatic 5-6 membered ring system containing one or more heteroatoms, for example one to three heteroatoms, which may be the same or different, such as nitrogen, oxygen, and sulfur. Where possible, said heteroaryl ring may be linked to the adjacent radical through carbon or nitrogen. Examples of heteroaryl rings include but are not limited to furan, benzofuran, thiophene, pyrrole, thiazole, oxazole, isothiazole, isoxazole, imidazole, pyrazole, triazole, pyridine, and pyrimidine. Said heteroaryl group may be unsubstituted or substituted with one or more substituents as described for the aryl group above. The term “heteroaryl” also includes polycyclic ring systems having two or more cyclic rings in which two or more carbons or heteroatoms are common to two adjoining rings (the rings are “fused rings”) wherein at least one of the rings is a heteroaryl as defined above whereas the other cyclic rings may be cycloalkyls, cycloalkenyls, cycloalkynyls, aromatic rings and/or saturated, unsaturated or aromatic heterocycles.

The term “heterocycle” as used herein refers to a monocyclic ring containing one or more heteroatoms, for example one to three heteroatoms, which may be the same or different, such as nitrogen, oxygen, and sulfur. The remaining ring members are formed by carbon atoms. The heterocycle typically has 4 to 8 ring members and preferably 5 or 6 ring members. Unless specified otherwise, a heterocycle may be aromatic, partially or fully saturated. Unless specified otherwise, it may or may not contain permissible substituents as specified herein.

The term “heterocyclic spiro” as used herein refers to a spirocyclic ring structure e.g. a bicyclic structure containing one or more heteroatoms, for example one to three heteroatoms, which may be the same or different, such as nitrogen, oxygen, and sulfur. The remaining ring members are formed by carbon atoms. The heterocyclic spiro typically has 7 to 11 ring members and preferably 7 or 9 ring members.

Unless specified otherwise, a heterocyclic spiro may be partially or fully saturated. Unless specified otherwise, it may or may not contain permissible substituents as specified herein.

The terms “hydroxy” and “hydroxyl” as used herein refer to the radical —OH.

The term “nitro” is art-recognized and refers to —NO₂; the term “sulfhydryl” is art-recognized and refers to —SH; and the term “sulfonyl” is art-recognized and refers to —SO₂—.

The definition of each expression, when it occurs more than once in any structure, is intended to be independent of its definition elsewhere in the same structure.

The terms “triflyl”, “tosyl”, “mesyl”, and “nonaflyl” are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate, p-toluenesulfonate, methanesulfonate, and nonafluorobutanesulfonate functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

The “pKa” of an amino group, as referred to herein, is intended to characterize the equilibrium of the following acid-base reaction

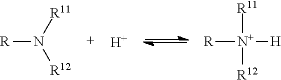

as follows: pKa=−log₁₀ (Ka)

wherein Ka=[c(R—N(R¹¹)(R¹²))*c(H⁺)]/c(R—NH(R¹¹)(R¹²)⁺)

with c( ) denoting the concentration in mol/I of the component specified in parentheses and wherein the unit mol/I of the equilibrium constant Ka is omitted when calculating its logarithm. The pKa value of an amino group may be determined by half-titrating the compound (such that c(R—N(R¹¹)(R¹²))=c(R—NH(R¹¹)(R¹²)⁺) and determining the pH. According to the Henderson-Hasselbalch equation, pKa=pH at that point of the titration, so that the pKa is obtained by the determination of pH. According to one embodiment of the invention, pKa may be determined by calculation relying on the corresponding feature within the chemical properties (standard parameters) of the software ChemDraw Professional, version 17.1.0.105.

The term “prodrug” refers to a derivative of an active compound (drug) that undergoes a transformation under the conditions of use, such as within the body, to release the active drug.

Prodrugs are frequently, but not necessarily, pharmacologically inactive until converted into the active drug.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above, e.g. in connection with substituted alkyls. The permissible substituents may be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. In this context, the term “permissible substituents” means any substituent that can be bonded to the core molecule without contravening general principles of chemical bond formation such as the maximum number of valence electrons for an atom of interest, and without making the compound so toxic for the patient that inacceptable toxicity is found even at the minimum dosage required for achieving a therapeutic effect.

For purposes of this invention, the chemical elements are identified in accordance with the Periodic Table of the Elements, CAS version, Handbook of Chemistry and Physics, 67th Ed., 1986-87, inside cover.

Also for purposes of the disclosure, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds that may be substituted or unsubstituted.

The term “pharmaceutically-acceptable salts” is art-recognized and refers to the relatively non-toxic, inorganic and organic acid addition salts, or inorganic or organic base addition salts of compounds, including, for example, those contained in compositions of the present invention, and including those present in other approved drugs (wherein approval may be by any competent authority in the EU, USA, CA, JP, CN or KR at date up to the effective date of the present application).

The term “diastereomerically pure” indicates that a chiral compound having two or more chiral centers is provided such that 90% or more, preferably 95% or more, of the molecules are in one diastereomeric configuration and 10% or less, preferably 5% or less, of the molecules are in other diastereomeric configurations. Likewise, the indication “enantiomerically pure” indicates that 90% or more, preferably 95% or more, of the molecules are present in the form of one of the enantiomers and 10% or less, preferably 5% or less, are present in the form of the other enantiomer (i.e. the compound has an enantiomeric excess of 80% ee or more and preferably 90% ee or more). The indication of a compound by means of a structural formula including information on the stereochemistry indicates that the compound is diastereomerically pure or enantiomerically pure in accordance with the above definitions.

A verbal statement that a compound is diastereomerically pure in connection with a structural formula not showing information on the stereochemistry of one of the chiral atoms indicates that said chiral atom with no information on the stereochemistry may be present in either one of the two possible configurations, but with the proviso that the one of the possible configurations must be predominant such that the above criteria are fulfilled, i.e. one diastereomer being present in a relative amount of 90% or more or preferably 95% or more.

The term “treating” includes any significant effect, e.g., lessening, reducing, modulating, or eliminating, that results in the improvement of the condition, disease, disorder and the like.

The term “prophylactic” or “therapeutic” treatment is art-recognized and refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

A “patient,” “subject” or “host” to be treated by the subject method may mean either a human or non-human animal. Non-human animals include companion animals (e.g. cats, dogs) and animals raised for consumption (i.e. food animals), such as cows, pigs, chickens. Non-human animals are preferably mammals.

The term “mammal” is known in the art, and exemplary mammals include humans, primates, bovines, porcines, canines, felines, and rodents (e.g., mice and rats).

The term “bioavailable” is art-recognized and refers to a form of the subject disclosure that allows for it, or a portion of the amount administered, to be absorbed by, incorporated to, or otherwise physiologically available to a subject or patient to whom it is administered.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as dextrose, lactose, glucose and sucrose; (2) starches, such as corn starch and potato starch as well as starch derivatives such as cyclodextrins and modified cyclodextrins including preferably (2-hydroxypropyl)-β-cyclodextrin and sulfobutylether-3-cyclodextrin; (3) cellulose, and its derivatives, such as microcrystalline cellulose, sodium carboxymethyl cellulose, methyl cellulose, ethyl cellulose, hydroxypropylmethyl cellulose (HPMC), and cellulose acetate; (4) powdered tragacanth; (5) malt; (6) gelatin; (7) talc; (8) matrix-forming polymeric excipients such as polyvinyl pyrrolidine (PVP), e.g. PVP K30, acrylic polymers and co-polymers such as the different grades of Eudragit and preferably Eurdragit L100, hydroxypropylmethyl cellulose acetate succinate (HPMCAS), other copolymers such as polyethylene glycol-based copolymers like Soluplus; (9) excipients, such as cocoa butter and suppository waxes; (10) oils, such as peanut oil, cottonseed oil, safflower oil, sesame oil, olive oil, corn oil and soybean oil; (11) glycols, such as propylene glycol; (12) polyols, such as glycerin, sorbitol, mannitol and polyethylene glycol; (13) esters, such as ethyl oleate, glyceryl behenate and ethyl laurate; (14) agar; (15) buffering agents, such as magnesium hydroxide and aluminum hydroxide; (16) alginic acid; (17) pyrogen-free water; (18) isotonic saline; (19) Ringer's solution; (20) ethyl alcohol; (21) phosphate buffer solutions; and (22) other non-toxic compatible substances employed in pharmaceutical formulations. The disclosed excipients may serve more than one function.

For example, fillers or binders may also be disintegrants, glidants, anti-adherents, lubricants, sweeteners and the like.

The term “solvent” is used herein to mean a liquid chemical substance that is capable of dissolving a significant quantity of another substance of interest, the “solute”, to thereby generate a clear homogeneous solution. The term “significant quantity” is determined by the intended use of the solution in such a manner that the intended use must be possible by the dissolved quantity of the solute. For instance, if it is intended to administer a compound of the present invention in the form of a solution by injection, the solvent must be capable of dissolving the compound in such amounts, to make administration of a therapeutic dose possible.

The terms “acid” and “base” are used to have their conventional meanings as proton donators and proton acceptors, respectively (i.e. Broensted acids and bases). A “strong base” is meant to be any base having a basicity of t-BuOK in THF or stronger. A “mild acid” is meant to be any acid having acidity of 1M H₂SO₄ or weaker.

Unless specified otherwise, all reactions described herein are carried out at reaction temperatures that yield the desired target compound and that provide a reasonable compromise between reaction rate and selectivity. Typical reaction temperatures for Pd-based coupling reactions and Fe-based cyclization reactions are 80° C. to 90° C. while removal of protecting groups is typically accomplished at a temperature of from 0° C. to room temperature (25° C.).

Unless specified otherwise, all indications in dependent claims that variable groups are the same as specified for the compound of formula I and its specific embodiments of formulae la and Ib, are to be understood such that the more specific meanings described for these variable groups in other dependent claims, are also possible and even preferred. The same applies to the description of meanings of variable groups in the general description. It is particularly preferred to rely on a combination of meanings for the different variable groups, wherein two, three or more and ideally all of these meanings are individually described as being preferred.

Unless specified otherwise, the term “protective group” is used herein to characterize a group that is bonded to a functional group to prevent this functional group from participating in a contemplated chemical reaction. The protective group must be inert under the conditions of the contemplated chemical reaction, but it must be possible to remove the protective group from the compound such that no further transformations take place in other parts of the molecule. Suitable protective groups are described for each functional group in “Greene's Protective Groups in Organic Synthesis”, Peter G. M. Wuts, Theodora W. Greene, John Wiley & Sons, 20 Dec. 2012.

### Overview

Surprisingly, it has been found that antibacterial activity against Gram-positive and/or Gram-negative bacteria, and more specifically S. aureus, E. coli, K. pneumoniae and/or A. baumannii, may be accomplished with a compound of formulae (1) and/or (Ia) as described herein. It has also surprisingly been found that a compound of the present invention may have a low MIC with respect to Gram-positive and/or Gram-negative bacteria and more specifically S. aureus, E. coli, K. pneumoniae and/or A. baumannii, indicating that a compound of formulae (1) and/or (Ia) may not only be effective against these types of bacteria, but may also be effective in low dosages which can thereby minimize side effects. Without wishing to be bound by theory, the inventors believe that the compounds of the invention may work through the mechanism of Fabl inhibition and, with respect to previous generations of Fabl inhibitor compounds and Gram-negative bacteria such as A. baumannii, E. coli, K. pneumoniae, may be better able to penetrate the cytoplasm of such bacteria and or may be less prone to efflux from said bacteria and/or may be more potent.

More specifically, in a first aspect, hereinafter referred to as first and second embodiment and as embodiment (A) in the appended claims, the invention relates to compounds of formula (1), wherein the amino group at the right-hand-side of the molecule is substituted with one or two small alkyl substituents or is incorporated into a small heterocycle formed by connecting such small alkyl substituents. Surprisingly, it has been found that such compounds exhibit particularly high affinity towards Fabl and especially A. Baumannii Fabl. Moreover, such compounds exhibit favourable lung exposure, which makes them particularly suitable for treating infections of the lung such as pneumonia and especially nosocomial pneumonia. According to a specific preferred aspect, the compounds of the first aspect have an amino group at the right-hand-side of the molecule that is substituted such that it exhibits a degree of basicity within a specified range. More specifically, the substituents of the amino group are selected such that the pKa of the amino group falls into the range of from 6.0 to 8.5, preferably 6.2 to 7.5 and more preferably 6.4 to 7.0; A surprisingly high affinity towards Fabl and especially A. Baumannii Fabl can be accomplished with such compounds. The compounds of this aspect also exhibit favourable lung exposure.

In a further aspect, hereinafter referred to as third and fourth embodiment and as embodiment (B) in the appended claims, the present invention provides compounds having defined stereochemistry in the 7-membered heterocycle of the right-hand-side of the molecule. Surprisingly, it was found that compounds having this defined stereochemistry exhibit surprisingly high affinity towards Fabl and especially A. Baumannii Fabl.

### Compounds of the Invention

The present invention relates to compounds represented by the following general formula I

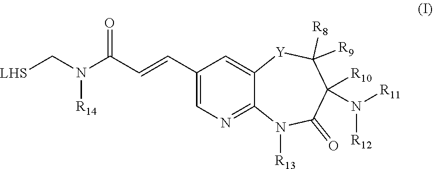

and pharmaceutically acceptable prodrugs, salts and/or solvates thereof, wherein LHS is

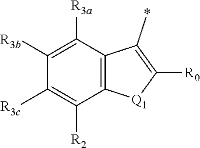

wherein, the asterisk (*) marks the point of attachment.

In a first embodiment, the variable groups have the following meanings:
Y is CH₂;
Q₁ is selected from the group consisting of O, S, NH and N—C₁₋₄-alkyl, preferably, Q₁ is O;
R₀ is selected from the group consisting of F, CH₂F, CH₃ and Cl, or alternatively R₀ together with R₁₄ form a heterocycle comprising the N to which R₁₄ is attached and having 5 to 8 ring members, wherein preferably the only heteroatom in said ring is the N to which R₁₄ is attached; it is preferred that R₀ is CH₃;

R₂ is selected from the group consisting of H, F, Cl, Br, I, C₁₋₄-alkyl, C₁₋₄-alkylene-OR₅, NR₅R₆, CO—NR₅R₆, C₁₋₄-alkylene-NR₅R₆, C₃₋₆-cycloalkyl, phenyl, and a heterocyclic group having 5 or 6 ring members and 1, 2 or 3 heteroatoms independently selected from N, O and S, wherein said C₁₋₄-alkyl, cycloalkyl, phenyl, or heterocyclic group may optionally be substituted with 1-3 R₇ groups; preferably R₂ is selected from the group consisting of H, F, Cl, Br, I, NR₅R₆, more preferably R₂ is H or F;

R₃ₐ, R_{3b} and R_{3c} are independently selected from the group consisting of H, F, Cl, Br, I, OH, NH₂, CH₃, preferably each of R₃ₐ, R_{3b} and R_{3c} is H;

R₅ and R₆ are independently selected from the group consisting of H, C₁₋₄-alkyl, C₃₋₆-cycloalkyl, phenyl, and a heterocyclic group having 5 or 6 ring members and 1, 2 or 3 heteroatoms independently selected from N, O and S, wherein said cycloalkyl, phenyl, or heterocyclic group may optionally be substituted with 1-3 R₇ groups; preferably R₅ is H while R₆ is selected from the group consisting of H, C₁₋₄-alkyl, C₃₋₆-cycloalkyl, phenyl, and a heterocyclic group having 5 or 6 ring members and 1, 2 or 3 heteroatoms independently selected from N, O and S, wherein said cycloalkyl, phenyl, or heterocyclic group may optionally be substituted with 1-3 R₇ groups;

R₇ is selected from the group consisting of H, F, I, Br, Cl, O, C₁₋₄-alkyl, CONH₂, OH, NH₂, O—C₁₋₄-alkyl, NH—C₁₋₄-alkyl, N(C₁₋₄-alkyl)₂, C₁₋₄-alkylene-OH, and C₁₋₄-alkylene-NH₂, NO₂, CN, C₂₋₄-alkenyl, C₂₋₄-alkynyl, C₂₋₄-alkynylene-OH, C₂₋₄-alkynylene-NH₂, SO₂CH₃, and O—C₁₋₄-alkylene-OH; preferably R₇ is selected from the group consisting of H, F, Br, Cl, C₁₋₄-alkyl, NH₂, O—C₁₋₄-alkyl, NH—C₁₋₄-alkyl, N(C₁₋₄-alkyl)₂, C₁₋₄-alkylene-OH, and C₁₋₄-alkylene-NH₂ and CN;

R₈ is H or F, preferably H and R₉ is selected from the group consisting of H, F, methyl, ethyl, CN, OH, NH₂ and CH₂—OH; preferably H, F, methyl, CN, OH, or CH₂—OH;

R₁₀ is H or methyl;

R₁₁ and R₁₂ are independently selected from the group consisting of H, R_{d}, optionally substituted C₁₋₄-alkyl, wherein each of the optionally substituted C₁₋₄-alkyl groups may carry a substituent selected from F, OH, OMe and NH₂, NHMe, NMe₂, or alternatively, R₁₁ and R₁₂together with the N to which they are attached form a heterocyclic group having 4, 5 or 6 ring members optionally comprising an oxygen atom or nitrogen atom in addition to the nitrogen atom bonded to the bicyclic group and carrying the R₁₁ and R₁₂ groups, wherein said heterocyclic group may carry one or two substituents independently selected from F, methyl, OH and NH₂, preferably a single substituent selected from F, methyl, OH and NH₂ or two methyl group substituents, which may be in geminal or different positions;

R₁₃ is selected from the group consisting of H or R_{d};

R₁₄ is CH₃, or alternatively R₁₄ together with R₀ of LHS form a heterocycle comprising the N to which R₁₄ is attached and having 5 to 8 ring members, wherein preferably the only heteroatom in said ring is the N to which R₁₄ is attached; preferably R₁₄ is CH₃;

and,

R_{d} is selected from the group consisting of —PO₃Rₑ₂, —CH₂—OPO₃Rₑ₂, wherein Rₑ is selected from the group consisting of H and a cation suitable for forming a pharmaceutically acceptable salt,

wherein the pharmaceutically acceptable prodrug is preferably a compound based on formula (1) and modified by attaching a methylene phosphate moiety or a phosphoramidate moiety;

wherein the compound of formula (1) is none of the following compounds:

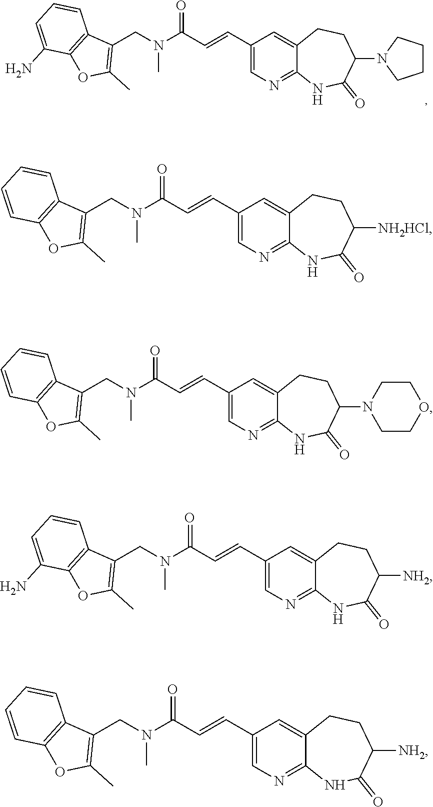

and neither any of the stereoisomers of these compounds such as

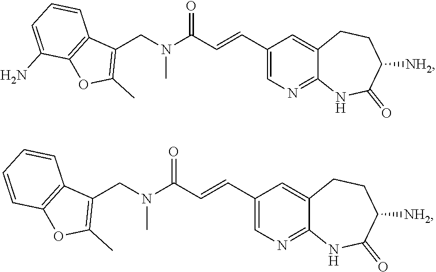 [SMILES: Cc1oc2c(N)cccc2c1CN(C)C(=O)/C=C/c1cnc2c(c1)CC[C@H](N)C(=O)N2 ; Cc1oc2ccccc2c1CN(C)C(=O)/C=C/c1cnc2c(c1)CC[C@H](N)C(=O)N2]

(E)-3-(7-amino-8-oxo-6,7,8,9-tetrahydro-5H-pyrido[2,3-b]azepin-3-yl)-N-methyl —N-((2-methyl benzofuran-3-yl)methyl)acrylamide,

(E)-N-methyl-N-((2-methylbenzofuran-3-yl)methyl)-3-(7-morpholino-8-oxo-6,7,8,9-tetrahydro-5H-pyrido[2,3-b]azepin-3-yl)acrylamide,

(E)-N-((7-Amino-2-methylbenzofuran-3-yl)methyl)-N-methyl-3-(8-oxo-7-(pyrrolidin-1-yl)-6,7,8,9-tetrahydro-5H-pyrido[2,3-b]azepin-3-yl)acrylamide,

(S,E)-N-((7-amino-2-methylbenzofuran-3-yl)methyl)-3-(7-amino-8-oxo-6,7,8,9-tetrahydro-5H-pyrido[2,3-b]azepin-3-yl)-N-methylacrylamide, and (S,E)-3-(7-amino-8-oxo-6,7,8,9-tetrahydro-5H-pyrido[2,3-b]azepin-3-yl)-N-methyl-N-((2-methylbenzofuran-3-yl)methyl)acrylamide.

In a preferred aspect of the first embodiment, R₂ is selected from the group consisting of H, F, Cl, Br, I, C₁₋₄-alkyl, ORs, C₁₋₄-alkylene-OR₅, CN, C₃₋₆-cycloalkyl, phenyl, and a heterocyclic group having 5 or 6 ring members and 1, 2 or 3 heteroatoms independently selected from N, O and S, wherein said C₁₋₄-alkyl, cycloalkyl, phenyl, or heterocyclic group may optionally be substituted with 1-3 R₇ groups. In a more preferred aspect of the first embodiment, R₂ is selected from the group consisting of H, F, Cl, Br and I. In a still more preferred aspect of the first embodiment, R₂ is H or F. In an even more preferred aspect of the first embodiment, R₂ is H.

According to a preferred aspect of the first embodiment, Q₁ is O, R₀ is CH₃, R₂ is H and each of R₃ₐ, R_{3b} and R_{3c} is H.

In a preferred aspect of the first embodiment, R₃ is H and R₉ is selected from the group consisting of H, F, CN, OH, or CH₂—OH.

According to another preferred aspect of the first embodiment, R₃ and R₉ are each H and R₁₀ is methyl. More specific compounds of this aspect are characterized not only by R₃ and R₉ each being H and R₁₀ being methyl, but additionally by R₁₁ and R₁₂each being H. Within this sub-genus of compounds with R₃ and R₉ each being H and R₁₀ being methyl, and optionally R₁₁ and R₁₂ each being H, R₁₃ is typically H and R₁₄ is typically methyl.

According to certain embodiments, R₃ₐ is H. According to some embodiments, R_{3b} is H. According to some embodiments, R_{3c} is H. According to some embodiments, R₉ is H, a hydroxyl group, a nitrile group or a methyl group. According to some embodiments, R₂ is H or F.

According to preferred aspects of the first embodiment, the compound of formula (I) is characterized by a formula (II) or, when taking the meaning for Y into account, formula (II-1), both of which are shown below:

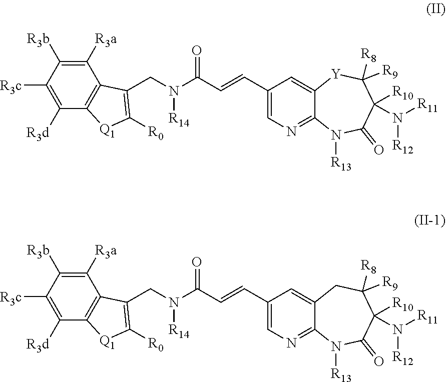

wherein the meanings of the variable groups are as specified above for the first embodiment. In specific aspects, two, three, four, five, six or more and most preferably all of the the variable groups have the following specific meanings, while the remaining variable groups are in accordance with the broader definitions specified above for the first embodiment:
(a) Q₁ is O;
(b) R₀ is CH₃;
(c) R₂ is selected from the group consisting of H, F, Cl, Br, I, NR₅R₆;
(d) each of R₃ₐ, R_{3b} and R_{3c} is H;
(e) R₅ is H;
(f) R₆ is selected from the group consisting of H, C₁₋₄-alkyl, C₃₋₆-cycloalkyl, phenyl, and a heterocyclic group having 5 or 6 ring members and 1, 2 or 3 heteroatoms independently selected from N, O and S, wherein said cycloalkyl, phenyl, or heterocyclic group may optionally be substituted with 1-3 R₇ groups;
(g) R₇ is selected from the group consisting of H, F, Br, Cl, C₁₋₄-alkyl, NH₂, O—C₁₋₄-alkyl, NH—C₁₋₄-alkyl, N(C₁₋₄-alkyl)₂, C₁₋₄-alkylene-OH, and C₁₋₄-alkylene-NH₂ and CN;
(h) R₅ is H;
(i) R₉ is selected from the group consisting of H, F, methyl, CN, OH, or CH₂—OH;
(j) R₁₁ and R₁₂ are independently selected from H, methyl, ethyl, 2-hydroxyethyl, 2-aminoethyl, or R₁₁ and R₁₂ together with the adjacent nitrogen atom form a heterocycle selected from an azetidine group, a pyrrolidine group, a piperidine group, a morpholine group and a piperazine group, wherein each of these heterocycles may be substituted by a group selected from F, OH, NH₂ and methyl or substituted by two methyl groups, which may be in geminal or different positions;
(k) R₁₄ is CH₃.

In certain preferred aspects, the compounds have at least the above-identified specific meanings (a), (b), (c), (d) and optionally one or more of (e) to (k).

In certain other preferred aspects, the compounds have at least the above-identified specific meanings (a), (b), (c), (d), (e) and optionally one or more of (f) to (k).

In certain other preferred aspects, the compounds have at least the above-identified specific meanings (a), (b), (c), (d), (e), (f) and optionally one or more of (g) to (k).

In certain other preferred aspects, the compounds have at least the above-identified specific meanings (a), (b), (c), (d), (e), (f), (g) and optionally one or more of (h) to (k).

In certain other preferred aspects, the compounds have at least the above-identified specific meanings (a), (b), (c), (d), (e), (f), (g), (h) and optionally one or more of (i) to (k).

In certain other preferred aspects, the compounds have at least the above-identified specific meanings (a), (b), (c), (d), (e), (f), (g), (h), (i) and optionally one or more of (j) to (k).

In certain other preferred aspects, the compounds have at least the above-identified specific meanings (a), (b), (c), (d), (e), (f), (g), (h), (i), (j) and optionally (k).

In certain other preferred aspects, the compounds have the above-identified specific meanings (b), (c), (d), (e), (f), (g), (h), (i), (j) and (k).

In certain other preferred aspects, the compounds have the above-identified specific meanings (a), (c), (d), (e), (f), (g), (h), (i), (j) and (k).

In certain other preferred aspects, the compounds have the above-identified specific meanings (a), (b), (d), (e), (f), (g), (h), (i), (j) and (k).

In certain other preferred aspects, the compounds have the above-identified specific meanings (a), (b), (c), (e), (f), (g), (h), (i), (j) and (k).

In certain other preferred aspects, the compounds have the above-identified specific meanings (a), (b), (c), (d), (f), (g), (h), (i), (j) and (k).

In certain other preferred aspects, the compounds have the above-identified specific meanings (a), (b), (c), (d), (e), (g), (h), (i), (j) and (k).

In certain other preferred aspects, the compounds have the above-identified specific meanings (a), (b), (c), (d), (e), (f), (h), (i), (j) and (k).

In certain other preferred aspects, the compounds have the above-identified specific meanings (a), (b), (c), (d), (e), (f), (g), (i), (j) and (k).

In certain other preferred aspects, the compounds have the above-identified specific meanings (a), (b), (c), (d), (e), (f), (g), (h), (j) and (k).

In certain other preferred aspects, the compounds have the above-identified specific meanings (a), (b), (c), (d), (e), (f), (g), (h), (i) and (k).

In certain other preferred aspects, the compounds have the above-identified specific meanings (a), (b), (c), (d), (e), (f), (g), (h), (i) and (j).

Particularly preferred are those of the above-identified compounds in which the meaning for R₂ under item (c) is either H or F.

In preferred aspects of the first embodiment, R₁₁ and R₁₂ are selected such that the nitrogen, to which R₁₁ and R₁₂ are attached, exhibits a pKa in the range of from 6.0 to 8.5, preferably 6.2 to 7.5 and more preferably 6.4 to 7.0; According to further preferred aspects of the first embodiment, the chiral ring atom carrying the NR₁₁R₁₂ group has a defined stereochemistry. That is, the present invention also relates to compounds in which 90% or more, preferably 95% or more, of the molecules are characterized by a configuration wherein the NR₁₁R₁₂ group is beneath the plane of the heterocyclic ring, as shown in the following formula (Ia):

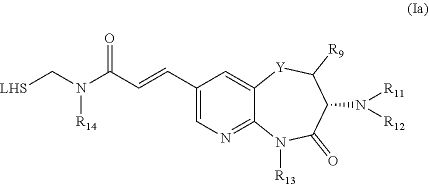

wherein LHS, Y, R₉, R₁₁, R₁₂, R₁₃ and R₁₄ are as specified above for the first embodiment. In relation to the preferred aspect of formula (II), this means that the compounds are characterized by the following formula (IIa):

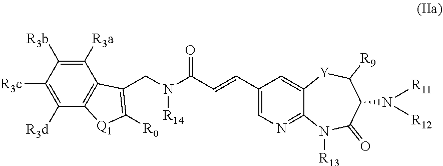

wherein the variable groups have the meanings specified for the first embodiment above, and especially the meanings specified as being preferred. Having regard to the meaning of Y, the formula can be expressed as formula (IIa-1), as shown below:

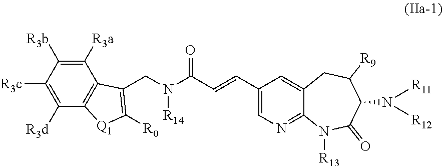

According to further advantageous aspects of the first embodiment, the stereochemistry of the chiral carbon carrying R₉ is also defined. According to preferred embodiments, the chiral ring atom carrying the NR₁₁R₁₂ group and the adjacent carbon carrying R₉ have defined stereochemistry such that the compound is diastereomerically pure. The present invention thus relates specifically to the following preferred compounds of formulae (Ib) and (Ic) wherein at least 90% of the molecules, preferably 95% of the molecules are characterized by the stereochemistry shown for formula (Ib) or (Ic) below.

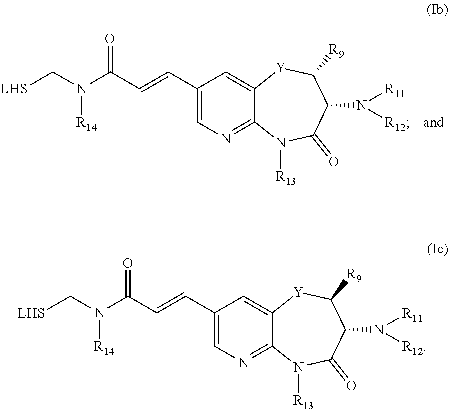

wherein the variable groups have the meanings specified for the first embodiment above, and especially the meanings specified as being preferred.

In a second embodiment, the variable groups have the following meanings:

Y is selected from the group consisting of NH, and NR_{d};

Q₁ is selected from the group consisting of O, S, NH and N—C₁₋₄-alkyl, preferably, Q₁ is O;

R₀ is selected from the group consisting of F, CH₂F, CH₃ and Cl, or alternatively R₀ together with R₁₄ form a heterocycle comprising the N to which R₁₄ is attached and having 5 to 8 ring members, wherein preferably the only heteroatom in said ring is the N to which R₁₄ is attached; it is preferred that R₀ is CH₃;

R₂ is selected from the group consisting of H, F, Cl, Br, I, C₁₋₄-alkyl, C₁₋₄-alkylene-OR₅, NR₅R₆, CO—NR₅R₆, C₁₋₄-alkylene-NR₅R₆, C₃₋₆-cycloalkyl, phenyl, and a heterocyclic group having 5 or 6 ring members and 1, 2 or 3 heteroatoms independently selected from N, O and S, wherein said C₁₋₄-alkyl, cycloalkyl, phenyl, or heterocyclic group may optionally be substituted with 1-3 R₇ groups; preferably R₂ is selected from the group consisting of H, F, Cl, Br, I, NR₅R₆, more preferably R₂ is H or F;

R₃ₐ, R_{3b} and R_{3c} are independently selected from the group consisting of H, F, Cl, Br, I, OH, NH₂, CH₃; preferably each of R₃ₐ, R_{3b} and R_{3c} is H;

R₅ and R₆ are independently selected from the group consisting of H, C₁₋₄-alkyl, C₃₋₆-cycloalkyl, phenyl, and a heterocyclic group having 5 or 6 ring members and 1, 2 or 3 heteroatoms independently selected from N, O and S, wherein said cycloalkyl, phenyl, or heterocyclic group may optionally be substituted with 1-3 R₇ groups; preferably R₅ is H while R₆ is selected from the group consisting of H, C₁₋₄-alkyl, C₃₋₆-cycloalkyl, phenyl, and a heterocyclic group having 5 or 6 ring members and 1, 2 or 3 heteroatoms independently selected from N, O and S, wherein said cycloalkyl, phenyl, or heterocyclic group may optionally be substituted with 1-3 R₇ groups;

R₇ is selected from the group consisting of H, F, I, Br, Cl, 0, C₁₋₄-alkyl, CONH₂, OH, NH₂, O—C₁₋₄-alkyl, NH—C₁₋₄-alkyl, N(C₁₋₄-alkyl)₂, C₁₋₄-alkylene-OH, and C₁₋₄-alkylene-NH₂, NO₂, CN, C₂₋₄-alkenyl, C₂₋₄-alkynyl, C₂₋₄-alkynylene-OH, C₂₋₄-alkynylene-NH₂, SO₂CH₃, and O—C₁₋₄-alkylene-OH; preferably R₇ is selected from the group consisting of H, F, Br, Cl, C₁₋₄-alkyl, NH₂, O—C₁₋₄-alkyl, NH—C₁₋₄-alkyl, N(C₁₋₄-alkyl)₂, C₁₋₄-alkylene-OH, and C₁₋₄-alkylene-NH₂ and CN;

R₈ is H or F and preferably H and R₉ is selected from the group consisting of H, F, methyl, ethyl, CN, OH, NH₂ and CH₂—OH; preferably H, methyl, CN, or CH₂—OH; R₁₀ is H or methyl;

R₁₁ and R₁₂ are independently selected from the group consisting of H, R_{d}, optionally substituted C₁₋₄-alkyl, wherein each of the optionally substituted C₁₋₄-alkyl groups may carry a substituent selected from F, OH, OMe and NH₂, NHMe, NMe₂, or alternatively, R₁₁ and R₁₂together with the N to which they are attached form a heterocyclic group having 4, 5 or 6 ring members optionally comprising an oxygen atom or nitrogen atom in addition to the nitrogen atom bonded to the bicyclic group and carrying the R₁₁ and R₁₂ groups, wherein said heterocyclic group may carry one or two substituents independently selected from F, methyl, OH and NH₂, preferably a single substituent selected from F, methyl, OH and NH₂ or two methyl group substituents, which may be in geminal or different positions;

R₁₃ is selected from the group consisting of H or R_{d};

R₁₄ is CH₃, or alternatively R₁₄ together with R₀ of LHS form a heterocycle comprising the N to which R₁₄ is attached and having 5 to 8 ring members, wherein preferably the only heteroatom in said ring is the N to which R₁₄ is attached; preferably R₁₄ is CH₃;

and,

R_{d} is selected from the group consisting of —PO₃Rₑ₂, —CH₂—OPO₃Rₑ₂, wherein Rₑ is selected from the group consisting of H and a cation suitable for forming a pharmaceutically acceptable salt, wherein the compound of formula (1) is none of the following compounds:

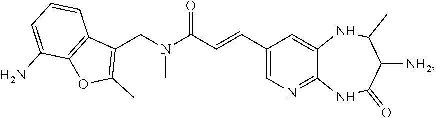 [SMILES: Cc1oc2c(N)cccc2c1CN(C)C(=O)/C=C/c1cnc2c(c1)NC(C)C(N)C(=O)N2]

and neither any of the stereoisomers of this compound such as

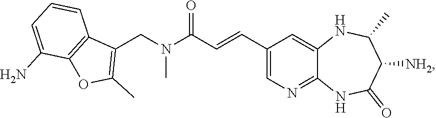 [SMILES: Cc1oc2c(N)cccc2c1CN(C)C(=O)/C=C/c1cnc2c(c1)N[C@H](C)[C@H](N)C(=O)N2]

(E)-3-((2R,3S)-3-Amino-2-methyl-4-oxo-2,3,4,5-tetrahydro-1H-pyrido[2,3-b][1,4]diazepin-8-yl)-N-((7-amino-2-methylbenzofuran-3-yl)methyl)-N-methylacrylamide.

In a preferred aspect of the second embodiment, R₂ is selected from the group consisting of H, F, Cl, Br, I, C₁₋₄-alkyl, ORs, C₁₋₄-alkylene-OR₅, CN, C₃₋₆-cycloalkyl, phenyl, and a heterocyclic group having 5 or 6 ring members and 1, 2 or 3 heteroatoms independently selected from N, O and S, wherein said C₁₋₄-alkyl, cycloalkyl, phenyl, or heterocyclic group may optionally be substituted with 1-3 R₇ groups.

In a more preferred aspect of the second embodiment, R₂ is selected from the group consisting of H, F, Cl, Br and I. In a still more preferred aspect of the second embodiment, R₂ is H or F. In an even more preferred aspect of the second embodiment, R₂ is H.

According to a preferred aspect of the second embodiment, Q₁ is O, R₀ is CH₃, R₂ is H and each of R₃ₐ, R_{3b} and R_{3c} is H.

According to certain embodiments, R₃ₐ is H. According to some embodiments, R_{3b} is H. According to some embodiments, R_{3c} is H. According to some embodiments, R₉ is H, a hydroxyl group, a nitrile group or a methyl group. According to some embodiments, R₂ is H or F.

According to preferred aspects of the second embodiment, the compound of formula (I) is characterized by a formula (II) or, preferably, formula (11-2), or as a prodrug formula (11-2′), all of which are shown below:

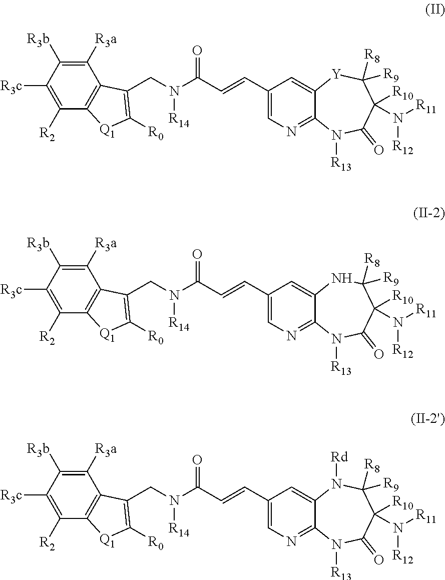

wherein the meanings of the variable groups are as specified above for the second embodiment. |n specific aspects, two, three, four, five, six or more and most preferably all of the the variable groups have following preferred meanings:
(a′) Q₁ is O;
(b′) R₀ is CH3;
(c′) R₂ is selected from the group consisting of H, F, Cl, Br, I, NR₅R₆;
(d′) each of R₃ₐ, R_{3b} and R_{3c} is H;
(e′) R₅ is H;
(f′) R₆ is selected from the group consisting of H, C₁₋₄-alkyl, C₃₋₆-cycloalkyl, phenyl, and a heterocyclic group having 5 or 6 ring members and 1, 2 or 3 heteroatoms independently selected from N, O and S, wherein said cycloalkyl, phenyl, or heterocyclic group may optionally be substituted with 1-3 R₇ groups;
(g′) R₇ is selected from the group consisting of H, F, Br, Cl, C₁₋₄-alkyl, NH₂, O—C₁₋₄-alkyl, NH—C₁₋₄-alkyl, N(C₁₋₄-alkyl)₂, C₁₋₄-alkylene-OH, and C₁₋₄-alkylene-NH₂ and CN;
(h′) R₃ is H;
(i′) R₉ is selected from the group consisting of H, methyl, CN, or CH₂—OH;
(j′) R₁₁ and R₁₂ are independently selected from H, methyl, ethyl, 2-hydroxyethyl, 2-aminoethyl, or R₁₁ and R₁₂ together with the adjacent nitrogen atom form a heterocycle selected from an azetidine group, a pyrrolidine group, a piperidine group, a morpholine group and a piperazine group, wherein each of these heterocycles may be substituted by a group selected from F, OH, NH₂ and methyl or substituted by two methyl groups, which may be in geminal or different positions; (k′) R₁₄ is CH₃.

In certain preferred aspects, the compounds have at least the above-identified specific meanings (a′), (b′), (c′), (d′) and optionally one or more of (e′) to (k′).

In certain other preferred aspects, the compounds have at least the above-identified specific meanings (a′), (b′), (c′), (d′), (e′) and optionally one or more of (f′) to (k′).

In certain other preferred aspects, the compounds have at least the above-identified specific meanings (a′), (b′), (c′), (d′), (e′), (f′) and optionally one or more of (g′) to (k′).

In certain other preferred aspects, the compounds have at least the above-identified specific meanings (a′), (b′), (c′), (d′), (e′), (f′), (g′) and optionally one or more of (h′) to (k′).

In certain other preferred aspects, the compounds have at least the above-identified specific meanings (a′), (b′), (c′), (d′), (e′), (f′), (g′), (h′) and optionally one or more of (i′) to (k′).

In certain other preferred aspects, the compounds have at least the above-identified specific meanings (a′), (b′), (c′), (d′), (e′), (f′), (g′), (h′), (i′) and optionally one or more of (j′) to (k′).

In certain other preferred aspects, the compounds have at least the above-identified specific meanings (a′), (b′), (c′), (d′), (e′), (f′), (g′), (h′), (i′), (j′) and optionally (k′).

In certain other preferred aspects, the compounds have the above-identified specific meanings (b′), (c′), (d′), (e′), (f′, (g′), (h′), (i′), (j′) and (k′).

In certain other preferred aspects, the compounds have the above-identified specific meanings (a′), (c′), (d′), (e′), (f), (g′), (h′), (i′), (j′) and (k′).

In certain other preferred aspects, the compounds have the above-identified specific meanings (a′), (b′), (d′), (e′), (f′), (g′), (h′), (i′), (j′) and (k′).

In certain other preferred aspects, the compounds have the above-identified specific meanings (a′), (b′), (c′), (e′), (f), (g′), (h′), (i′), (j′) and (k′).

In certain other preferred aspects, the compounds have the above-identified specific meanings (a′), (b′), (c′), (d′), (f′), (g′), (h′), (i′), (j′) and (k′).

In certain other preferred aspects, the compounds have the above-identified specific meanings (a′), (b′), (c′), (d′), (e′), (g′), (h′), (i′), (j′) and (k′).

In certain other preferred aspects, the compounds have the above-identified specific meanings (a′), (b′), (c′), (d′), (e′), (f′), (h′), (i′), (j′) and (k′).

In certain other preferred aspects, the compounds have the above-identified specific meanings (a′), (b′), (c′), (d′), (e′), (f, (g′), (i′), (j′) and (k′).

In certain other preferred aspects, the compounds have the above-identified specific meanings (a′), (b′), (c′), (d′), (e′), (f′), (g′), (h′), (j′) and (k′).

In certain other preferred aspects, the compounds have the above-identified specific meanings (a′), (b′), (c′), (d′), (e′), (f′), (g′), (h′), (i′) and (k′).

In certain other preferred aspects, the compounds have the above-identified specific meanings (a′), (b′), (c′), (d′), (e′), (f′), (g′), (h′), (i′) and (j′).

Particularly preferred are those of the above-identified compounds in which the meaning for R₂ under item (c′) is either H or F.

In preferred aspects of the second embodiment, R₁₁ and R₁₂ are selected such that the nitrogen, to which R₁₁ and R₁₂ are attached, exhibits a pKa in the range of from 6.0 to 8.5, preferably 6.2 to 7.5 and more preferably 6.4 to 7.0; According to further preferred aspects of the second embodiment, the chiral ring atom carrying the NR₁₁R₁₁ group has a defined stereochemistry. That is, the present invention also relates to compounds in which 90% or more, preferably 95% or more, of the molecules are characterized by a configuration wherein the NR₁₁R₁₂ group is beneath the plane of the heterocyclic ring, as shown in the following formula (Ia):

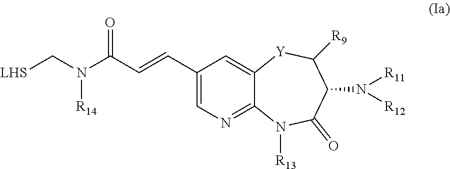

wherein LHS, Y, R₉, R₁₁, R₁₂, R₁₃ and R₁₄ are as specified above for the second embodiment. In relation to the preferred aspect of formula (II), this means that the compounds are characterized by the following formula (IIa-2) or, for the prodrug aspect, formula (IIa-2′):

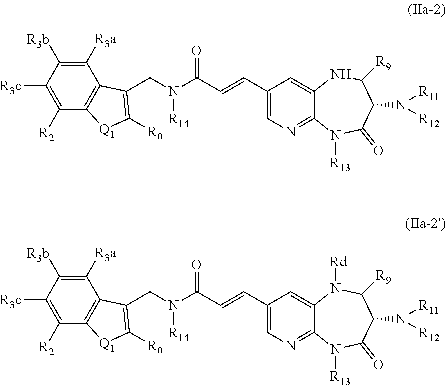

wherein the variable groups have the meanings specified for the second embodiment above, and especially the meanings specified as being preferred

According to further advantageous aspects of the second embodiment, the stereochemistry of the chiral carbon carrying R₉ is also defined. According to preferred embodiments, the chiral ring atom carrying the NR₁₁R_{1R} group and the adjacent carbon carrying R₉ have defined stereochemistry such that the compound is diastereomerically pure. The present invention thus relates specifically to the following preferred compounds of formulae (Ib) and (Ic) wherein at least 90% of the molecules, preferably 95% of the molecules are characterized by the stereochemistry shown for formula (Ib) or (Ic) below.

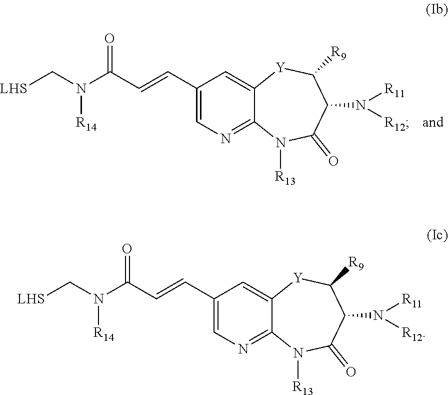

wherein the variable groups have the meanings specified for the second embodiment above, and especially the meanings specified as being preferred.

In a third embodiment, the compound of formula (1) is a compound characterized by formula (Ia)

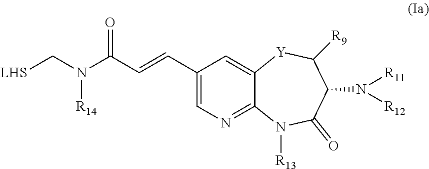

or a pharmaceutically acceptable prodrug, salt and/or solvate thereof; LHS is

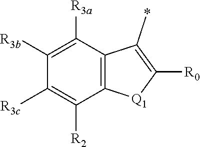

wherein, the asterisk (*) marks the point of attachment;

Y is CH₂;

Q₁ is selected from the group consisting of O, S, NH and N—C₁₋₄-alkyl; preferably Q₁ is O;

R₀ is selected from the group consisting of F, CH₂F, CH₃ and Cl, or alternatively R₀ together with R₁₄ form a heterocycle comprising the N to which R₁₄ is attached and having 5 to 8 ring members, wherein preferably the only heteroatom in said ring is the N to which R₁₄ is attached; it is preferred that R₀ is CH₃;

R₂ is selected from the group consisting of H, F, Cl, Br, I, C₁₋₄-alkyl, ORs, C₁₋₄-alkylene-OR₅, CN, NR₅R₆, CO—NR₅R₆, C₁₋₄-alkylene-NR₅R₆, C₃₋₆-cycloalkyl, phenyl, and a heterocyclic group having 5 or 6 ring members and 1, 2 or 3 heteroatoms independently selected from N, O and S, wherein said C₁₋₄-alkyl, cycloalkyl, phenyl, or heterocyclic group may optionally be substituted with 1-3 R₇ groups; preferably R₂ is selected from the group consisting of H, F, Cl, Br, I, NR₅R₆, more preferably R₂ is H or F;

R₃ₐ, R_{3b} and R_{3c} are independently selected from the group consisting of H, F, Cl, Br, I, OH, NH₂, CH₃; preferably each of R₃ₐ, R_{3b} and R_{3c} is H;

R₅ and R₆ are independently selected from is selected from the group consisting of H, C₁₋₄-alkyl, C₃₋₆-cycloalkyl, phenyl, and a heterocyclic group having 5 or 6 ring members and 1, 2 or 3 heteroatoms independently selected from N, O and S, wherein said cycloalkyl, phenyl, or heterocyclic group may optionally be substituted with 1-3 R₇ groups; preferably R₅ is H while R₆ is selected from the group consisting of H, C₁₋₄-alkyl, C₃₋₆-cycloalkyl, phenyl, and a heterocyclic group having 5 or 6 ring members and 1, 2 or 3 heteroatoms independently selected from N, O and S, wherein said cycloalkyl, phenyl, or heterocyclic group may optionally be substituted with 1-3 R₇ groups;

R₇ is selected from the group consisting of H, F, I, Br, Cl, O, C₁₋₄-alkyl, CONH₂, OH, NH₂, O—C₁₋₄-alkyl, NH—C₁₋₄-alkyl, N(C₁₋₄-alkyl)₂, C₁₋₄-alkylene-OH, and C₁₋₄-alkylene-NH₂, NO₂, CN, C₂₋₄-alkenyl, C₂₋₄-alkynyl, C₂₋₄-alkynylene-OH, C₂₋₄-alkynylene-NH₂, SO₂CH₃, and O—C₁₋₄-alkylene-OH; preferably R₇ is selected from the group consisting of H, F, Br, Cl, C₁₋₄-alkyl, NH₂, O—C₁₋₄-alkyl, NH—C₁₋₄-alkyl, N(C₁₋₄-alkyl)₂, C₁₋₄-alkylene-OH, and C₁₋₄-alkylene-NH₂ and CN;

R₉ is a methyl group, hydroxyl group or nitrile group;

R₁₁ and R₁₂ are independently selected from the group consisting of H, R_{d}, C₁₋₄-alkyl, CO—C₁₋₄-alkyl, SO₂(C₁₋₄-alkyl)₁, C₁₋₄-alkyl-F, C₁₋₄-alkylene-OH, and C₁₋₄-alkylene-NH₂, or alternatively, R₁₁ and R₁₂ together with the N to which they are attached form a heterocyclic group having 4 to 9 ring members and 1, 2 or 3 heteroatoms independently selected from N, O and S or R₁₁ and R₁₂ form a heterocyclic spiro group having 7 to 11 ring members and 1, 2 or 3 heteroatoms independently selected from N, O and S, wherein said heterocyclic or heterocyclic spiro group may be substituted with 1-3 R₇ groups, wherein the heterocyclic group formed by R₁₁ and R₁₂together with the N to which they are attached is preferably an optionally substituted heterocyclic group having 4, 5 or 6 ring members optionally comprising an oxygen atom or NH group in addition to the nitrogen atom carrying the R₁₁ and R₁₂ groups, wherein said optionally substituted heterocyclic group may carry one or two substituents independently selected from F, methyl, OH and NH₂, preferably a single substituent selected from F, methyl, OH and NH₂ or two methyl group substituents, which may be in geminal or different positions;

preferably R₁₁ and R₁₂ are independently selected from the group consisting of H, optionally substituted C₁₋₄-alkyl, wherein each of the optionally substituted C₁₋₄-alkyl groups may carry a substituent selected from F, OH, OMe and NH₂, NHMe, NMe₂, or alternatively, R₁₁ and R₁₂ together with the N to which they are attached form a heterocyclic group having 4, 5 or 6 ring members optionally comprising an oxygen atom or nitrogen atom in addition to the nitrogen atom bonded to the bicyclic group and carrying the R₁₁ and R₁₂ groups, wherein said heterocyclic group may carry one or two substituents independently selected from F, methyl, OH and NH₂, preferably a single substituent selected from F, methyl, OH and NH₂ or two methyl group substituents, which may be in geminal or different positions;

R₁₃ is selected from the group consisting of H or R_{d};

R₁₄ is CH₃, or alternatively R₁₄ together with R₀ of LHS form a heterocycle comprising the N to which R₁₄ is attached and having 5 to 8 ring members, wherein preferably the only heteroatom in said ring is the N to which R₁₄ is attached; preferably R₁₄ is CH₃;

and,

R_{d} is selected from the group consisting of —PO₃Rₑ₂, —CH₂—OPO₃Rₑ₂, wherein Rₑ is selected from the group consisting of H and a cation suitable for forming a pharmaceutically acceptable salt, and wherein the compound is diastereomerically pure in relation to the chiral carbon atom carrying R₉ and the chiral carbon atom carrying NR₁₁R₁₂.

In a preferred aspect of the third embodiment, R₂ is selected from the group consisting of H, F, Cl, Br, I, C₁₋₄-alkyl, ORs, C₁₋₄-alkylene-OR₅, CN, C₃₋₆-cycloalkyl, phenyl, and a heterocyclic group having 5 or 6 ring members and 1, 2 or 3 heteroatoms independently selected from N, O and S, wherein said C₁₋₄-alkyl, cycloalkyl, phenyl, or heterocyclic group may optionally be substituted with 1-3 R₇ groups. In a more preferred aspect of the third embodiment, R₂ is selected from the group consisting of H, F, Cl, Br and I. In a still more preferred aspect of the third embodiment, R₂ is H or F. In an even more preferred aspect of the third embodiment, R₂ is H.

In a preferred aspect of the third embodiment, R₉ is a hydroxyl group or nitrile group.

According to a preferred aspect of the third embodiment, Q₁ is O, R₀ is CH₃, R₂ is H and each of R₃ₐ, R_{3b} and R_{3c} is H.

According to certain embodiments, R₃ₐ is H or F. According to some embodiments, R_{3b} is H. According to some embodiments, R_{3c} is H, F, OH or NH₂. According to some embodiments, R₉ is a hydroxyl group, a nitrile group or a methyl group. According to some embodiments, R₂ is H.

According to preferred aspects of the third embodiment, the compound of formula (Ia) is characterized by a formula (IIa) or, when taking the meaning for Y into account, formula (IIa-1), both of which are shown below:

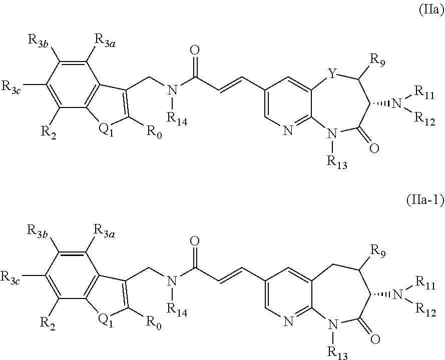

wherein the meanings of the variable groups are as specified above for the third embodiment.

More specifically, compounds according to this aspect are either compounds of the following formula (IIIb-1) or compounds of the following formula (IIc-1), as shown below, wherein the meanings of the variable groups are as specified above for the third embodiment:

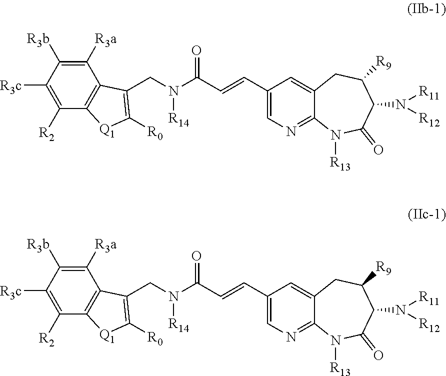

In specific aspects, two, three, four, five, six or more and most preferably all of the the variable groups have the specific meanings, while the remaining variable groups are in accordance with the broader definitions specified above for the third embodiment:
(a″) Q₁ is O;
(b″) R₀ is CH₃;
(c″) R₂ is selected from the group consisting of H, F, Cl, Br, I, NR₅R₆;
(d″) each of R₃ₐ, R_{3b} and R_{3c} is H;
(e″) R₅ is H;
(f″) R₆ is selected from the group consisting of H, C₁₋₄-alkyl, C₃₋₆-cycloalkyl, phenyl, and a heterocyclic group having 5 or 6 ring members and 1, 2 or 3 heteroatoms independently selected from N, O and S, wherein said cycloalkyl, phenyl, or heterocyclic group may optionally be substituted with 1-3 R₇ groups;
(g″) R₇ is selected from the group consisting of H, F, Br, Cl, C₁₋₄-alkyl, NH₂, O—C₁₋₄-alkyl, NH—C₁₋₄-alkyl, N(C₁₋₄-alkyl)₂, C₁₋₄-alkylene-OH, and C₁₋₄-alkylene-NH₂ and CN;
(h″)R₁₁ and R₁₂ are independently selected from H, methyl, ethyl, 2-hydroxyethyl, 2-aminoethyl, or R₁₁ and R₁₂ together with the adjacent nitrogen atom form a heterocycle selected from an azetidine group, a pyrrolidine group, a piperidine group, a morpholine group and a piperazine group, wherein each of these heterocycles may be substituted by a group selected from F, OH, NH₂ and methyl or substituted by two methyl groups, which may be in geminal or different positions;
(i″) R₁₄ is CH₃.

In certain preferred aspects, the compounds have at least the above-identified specific meanings (a″), (b″), (c″), (d″) and optionally one or more of (e″) to (i″).

In certain other preferred aspects, the compounds have at least the above-identified specific meanings (a″), (b″), (c″), (d″), (e″) and optionally one or more of (f″) to (i″).

In certain other preferred aspects, the compounds have at least the above-identified specific meanings (a″), (b″), (c″), (d″), (e″), (f″) and optionally one or more of (g″) to (i″).

In certain other preferred aspects, the compounds have at least the above-identified specific meanings (a″), (b″), (c″), (d″), (e″), (f″), (g″) and optionally one or more of (h″) to (i″).

In certain other preferred aspects, the compounds have at least the above-identified specific meanings (a″), (b″), (c″), (d″), (e″), (f″), (g″), (h″) and optionally (i″).

In certain other preferred aspects, the compounds have the above-identified specific meanings (b″), (c″), (d″), (e″), (f″), (g″), (h″) and (i″).

In certain other preferred aspects, the compounds have the above-identified specific meanings (a″), (c″), (d″), (e″), (f″), (g″), (h″) and (i″).

In certain other preferred aspects, the compounds have the above-identified specific meanings (a″), (b″), (d″), (e r), (f″), (g″), (h″) and (i″).

In certain other preferred aspects, the compounds have the above-identified specific meanings (a″), (b″), (c″), (e″), (f″), (g″), (h″) and (i″).

In certain other preferred aspects, the compounds have the above-identified specific meanings (a″), (b″), (c″), (d″), (f″), (g″), (h″) and (i″).

In certain other preferred aspects, the compounds have the above-identified specific meanings (a″), (b′), (c″), (d″), (e″), (g″), (h″) and (i″).

In certain other preferred aspects, the compounds have the above-identified specific meanings (a″), (b″), (c″), (d″), (e″), (f″), (h″) and (i″).

In certain other preferred aspects, the compounds have the above-identified specific meanings (a″), (b″), (c″), (d″), (e″), (f′), (g″) and (i″).

In certain other preferred aspects, the compounds have the above-identified specific meanings (a″), (b″), (c″), (d″), (e″), (f″), (g″) and (h″).

Particularly preferred are those of the above-identified compounds in which the meaning for R₂ under item (c″) is either H or F.

In preferred aspects of the third embodiment, R₁₁ and R₁₂ are selected such that the nitrogen, to which R₁₁ and R₁₂ are attached, exhibits a pKa in the range of from 6.0 to 8.5, preferably 6.2 to 7.5 and more preferably 6.4 to 7.0;

In a fourth embodiment, the compound of formula (I) is a compound characterized by formula (Ia)

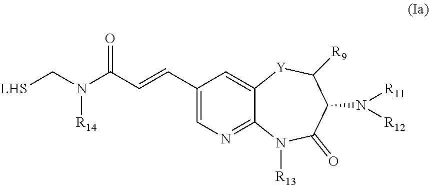

or a pharmaceutically acceptable prodrug, salt and/or solvate thereof; LHS is

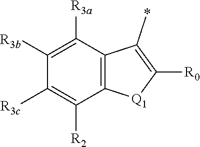

wherein, the asterisk (*) marks the point of attachment;

Y is NH or NR_{d};

Q₁ is selected from the group consisting of O, S, NH and N—C₁₋₄-alkyl; preferably Q₁ is O;

R₀ is selected from the group consisting of F, CH₂F, CH₃ and Cl, or alternatively R₀ together with R₁₄ form a heterocycle comprising the N to which R₁₄ is attached and having 5 to 8 ring members, wherein preferably the only heteroatom in said ring is the N to which R₁₄ is attached; it is preferred that R₀ is CH₃;

R₂ is selected from the group consisting of H, F, Cl, Br, I, C₁₋₄-alkyl, ORs, C₁₋₄-alkylene-OR₅, CN, NR₅R₆, CO—NR₅R₆, C₁₋₄-alkylene-NR₅R₆, C₃₋₆-cycloalkyl, phenyl, and a heterocyclic group having 5 or 6 ring members and 1, 2 or 3 heteroatoms independently selected from N, O and S, wherein said C₁₋₄-alkyl, cycloalkyl, phenyl, or heterocyclic group may optionally be substituted with 1-3 R₇ groups; preferably R₂ is selected from the group consisting of H, F, Cl, Br, I, NR₅R₆, more preferably R₂ is H or F;

R₃ₐ, R_{3b} and R_{3c} are independently selected from the group consisting of H, F, Cl, Br, I, OH, NH₂, CH₃; preferably each of R₃ₐ, R_{3b} and R_{3c} is H;

R₅ and R₆ are independently selected from is selected from the group consisting of H, C₁₋₄-alkyl, C₃₋₆-cycloalkyl, phenyl, and a heterocyclic group having 5 or 6 ring members and 1, 2 or 3 heteroatoms independently selected from N, O and S, wherein said cycloalkyl, phenyl, or heterocyclic group may optionally be substituted with 1-3 R₇ groups; preferably R₅ is H while R₆ is selected from the group consisting of H, C₁₋₄-alkyl, C₃₋₆-cycloalkyl, phenyl, and a heterocyclic group having 5 or 6 ring members and 1, 2 or 3 heteroatoms independently selected from N, O and S, wherein said cycloalkyl, phenyl, or heterocyclic group may optionally be substituted with 1-3 R₇ groups;

R₇ is selected from the group consisting of H, F, I, Br, Cl, O, C₁₋₄-alkyl, CONH₂, OH, NH₂, O—C₁₋₄-alkyl, NH—C₁₋₄-alkyl, N(C₁₋₄-alkyl)₂, C₁₋₄-alkylene-OH, and C₁₋₄-alkylene-NH₂, NO₂, CN, C₂₋₄-alkenyl, C₂₋₄-alkynyl, C₂₋₄-alkynylene-OH, C₂₋₄-alkynylene-NH₂, SO₂CH₃, and O—C₁₋₄-alkylene-OH; preferably R₇ is selected from the group consisting of H, F, Br, Cl, C₁₋₄-alkyl, NH₂, O—C₁₋₄-alkyl, NH—C₁₋₄-alkyl, N(C₁₋₄-alkyl)₂, C₁₋₄-alkylene-OH, and C₁₋₄-alkylene-NH₂ and CN;

R₉ is a methyl group, hydroxyl group or nitrile group; preferably R₉ is a methyl group or nitrile group;

R₁₁ and R₁₂ are independently selected from the group consisting of H, R_{d}, C₁₋₄-alkyl, CO—C₁₋₄-alkyl, SO₂(C₁₋₄-alkyl)1, C₁₋₄-alkyl-F, C₁₋₄-alkylene-OH, and C₁₋₄-alkylene-NH₂, or alternatively, R₁₁ and R₁₂ together with the N to which they are attached form a heterocyclic group having 4 to 9 ring members and 1, 2 or 3 heteroatoms independently selected from N, O and S or R₁₁ and R₁₂ form a heterocyclic spiro group having 7 to 11 ring members and 1, 2 or 3 heteroatoms independently selected from N, O and S, wherein said heterocyclic or heterocyclic spiro group may be substituted with 1-3 R₇ groups, wherein the heterocyclic group formed by R₁₁ and R₁₂together with the N to which they are attached is preferably an optionally substituted heterocyclic group having 4, 5 or 6 ring members optionally comprising an oxygen atom or NH group in addition to the nitrogen atom carrying the R₁₁ and R₁₂ groups, wherein said optionally substituted heterocyclic group may carry one or two substituents independently selected from F, methyl, OH and NH₂, preferably a single substituent selected from F, methyl, OH and NH₂ or two methyl group substituents, which may be in geminal or different positions;

preferably R₁₁ and R₁₂ are independently selected from the group consisting of H, optionally substituted C₁₋₄-alkyl, wherein each of the optionally substituted C₁₄-alkyl groups may carry a substituent selected from F, OH, OMe and NH₂, NHMe, NMe₂, or alternatively, R₁₁ and R₁₂ together with the N to which they are attached form a heterocyclic group having 4, 5 or 6 ring members optionally comprising an oxygen atom or nitrogen atom in addition to the nitrogen atom bonded to the bicyclic group and carrying the R₁₁ and R₁₂ groups, wherein said heterocyclic group may carry one or two substituents independently selected from F, methyl, OH and NH₂, preferably a single substituent selected from F, methyl, OH and NH₂ or two methyl group substituents, which may be in geminal or different positions;

R₁₃ is selected from the group consisting of H or R_{d};

R₁₄ is CH₃, or alternatively R₁₄ together with R₀ of LHS form a heterocycle comprising the N to which R₁₄ is attached and having 5 to 8 ring members, wherein preferably the only heteroatom in said ring is the N to which R₁₄ is attached;

and,

R_{d} is selected from the group consisting of —PO₃Rₑ₂, —CH₂—OPO₃Rₑ₂, wherein Rₑ is selected from the group consisting of H and a cation suitable for forming a pharmaceutically acceptable salt,

wherein the compound is diastereomerically pure in relation to the chiral carbon atom carrying R₉ and the chiral carbon atom carrying NR₁₁R₁₂;

wherein the compound of formula (Ia) is not

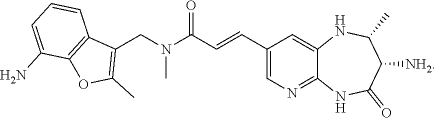 [SMILES: Cc1oc2c(N)cccc2c1CN(C)C(=O)/C=C/c1cnc2c(c1)N[C@H](C)[C@H](N)C(=O)N2]

In a preferred aspect of the fourth embodiment, R₂ is selected from the group consisting of H, F, Cl, Br, I, C₁₋₄-alkyl, ORs, C₁₋₄-alkylene-OR₅, CN, C₃₋₆-cycloalkyl, phenyl, and a heterocyclic group having 5 or 6 ring members and 1, 2 or 3 heteroatoms independently selected from N, O and S, wherein said C₁₋₄-alkyl, cycloalkyl, phenyl, or heterocyclic group may optionally be substituted with 1-3 R₇ groups. In a more preferred aspect of the fourth embodiment, R₂ is selected from the group consisting of H, F, Cl, Br and I. In a still more preferred aspect of the fourth embodiment, R₂ is H or F. In an even more preferred aspect of the fourth embodiment, R₂ is H.

According to a preferred aspect of the fourth embodiment, Q₁ is O, R₀ is CH₃, R₂ is H and each of R₃ₐ, R_{3b} and R_{3c} is H.

According to certain embodiments, R₃ₐ is H. According to some embodiments, R_{3b} is H. According to some embodiments, R_{3c} is H. According to some embodiments, R₉ is a nitrile group or a methyl group. According to some embodiments, R₂ is H or F.

According to preferred aspects of the fourth embodiment, the compound of formula (Ia) is characterized by a formula (IIa) as shown above, or, when taking the meaning for Y into account, formula (IIa-2), or, for the prodrug aspect, formula (IIa-2′), both of which are shown below:

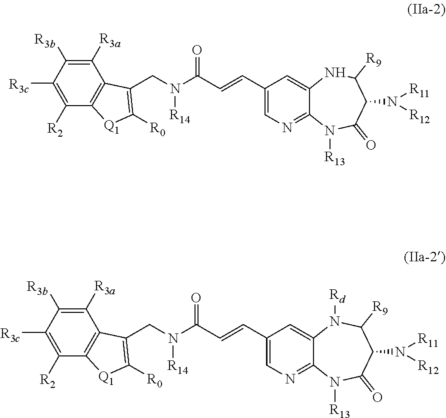

wherein the meanings of the variable groups are as specified above for the fourth embodiment.

More specifically, compounds according to this aspect are either compounds of the following formula (IIIb-2) or compounds of the following formula (IIc-2), as shown below, wherein the meanings of the variable groups are as specified above for the fourth embodiment:

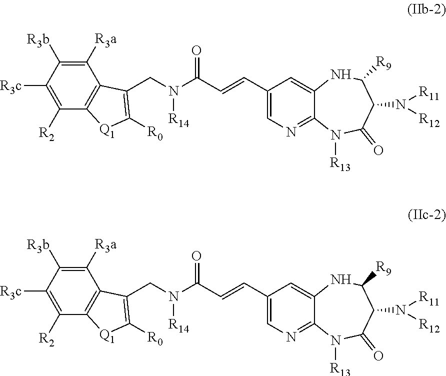

For the prodrug aspect, the corresponding structures are shown by the formulae (IIb-2′) and (IIc-2′) below:

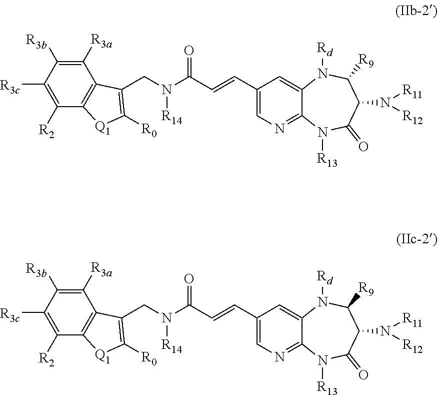

In specific aspects, two, three, four, five, six or more and most preferably all of the the variable groups have the following specific meanings, while the remaining variable groups are in accordance with the broader definitions specified above for the fourth embodiment:
(a″′) Q₁ is O;
(b″′) R₀ is CH₃;
(c″′) R₂ is selected from the group consisting of H, F, Cl, Br, I, NR₅R₆;
(d″′) each of R₃ₐ, R_{3b} and R_{3c} is H;
(e″′) R₅ is H;
(f″′) R₆ is selected from the group consisting of H, C₁₋₄-alkyl, C₃₋₆-cycloalkyl, phenyl, and a heterocyclic group having 5 or 6 ring members and 1, 2 or 3 heteroatoms independently selected from N, O and S, wherein said cycloalkyl, phenyl, or heterocyclic group may optionally be substituted with 1-3 R₇ groups;
(g″′) R₇ is selected from the group consisting of H, F, Br, Cl, C₁₋₄-alkyl, NH₂, O—C₁₋₄-alkyl, NH—C₁₋₄-alkyl, N(C₁₋₄-alkyl)₂, C₁₋₄-alkylene-OH, and C₁₋₄-alkylene-NH₂ and CN;
(h″′) R₉ is selected from the group consisting of methyl or CN;
(i″′) R₁₁ and R₁₂ are independently selected from H, methyl, ethyl, 2-hydroxyethyl, 2-aminoethyl, or R₁₁ and R₁₂ together with the adjacent nitrogen atom form a heterocycle selected from an azetidine group, a pyrrolidine group, a piperidine group, a morpholine group and a piperazine group, wherein each of these heterocycles may be substituted by a group selected from F, OH, NH₂ and methyl or substituted by two methyl groups, which may be in geminal or different positions;
(j″′) R₁₄ is CH₃.

In certain preferred aspects, the compounds have at least the above-identified specific meanings (a″′), (b′″), (c″′), (d″′) and optionally one or more of (e′″) to (j″′).

In certain other preferred aspects, the compounds have at least the above-identified specific meanings (a′″), (b′″), (c″′), (d″′), (e″′) and optionally one or more of (f′″) to (j″′).

In certain other preferred aspects, the compounds have at least the above-identified specific meanings (a′″), (b′″), (c″′), (d″′), (e″′), (f′″) and optionally one or more of (g′″) to (j″′).

In certain other preferred aspects, the compounds have at least the above-identified specific meanings (a′″), (b′″), (c″′), (d″′), (e″′), (f′″), (g″′) and optionally one or more of (h′″) to (j′″).

In certain other preferred aspects, the compounds have at least the above-identified specific meanings (a′″), (b′″), (c′″), (d′″), (e′″), (f′″), (g′″), (h′″) and optionally one or more of (i′″) to (j′″).

In certain other preferred aspects, the compounds have at least the above-identified specific meanings (a′″), (b′″), (c′″), (d′″), (e′″), (f′″), (g′″), (h′″), (i′″) and optionally (j′″).

In certain other preferred aspects, the compounds have the above-identified specific meanings (b″′), (c″′), (d′), (e″′), (f″′), (g″′), (h″′), (i″′) and (j″′).

In certain other preferred aspects, the compounds have the above-identified specific meanings (a″ (c″′), (d″′), (e″′), (f″′), (g″′), (h″′), (i″′) and (j″′).

In certain other preferred aspects, the compounds have the above-identified specific meanings (a″ (b″′), (d′″), (e″′), (f″′), (g″′), (h′″), (i″′) and (j″′).

In certain other preferred aspects, the compounds have the above-identified specific meanings (a″ (b″′), (c″), (e″′), (f″′), (g″′), (h″′), (i″′) and (j″′).

In certain other preferred aspects, the compounds have the above-identified specific meanings (a″ (b′″), (c′″), (d″′), (f′″), (g″′), (h″′), (i′″) and (j′″).

In certain other preferred aspects, the compounds have the above-identified specific meanings (a″ (b′″), (c′″), (d″′), (e′″), (g″′), (h′″), (i″′) and (j″′).

In certain other preferred aspects, the compounds have the above-identified specific meanings (a″ (b″′), (c″′), (d″′), (e′″), (f″′), (h″′), (i′″) and (j″′).

In certain other preferred aspects, the compounds have the above-identified specific meanings (a (b′″), (c′″), (d″′), (e′″), (f″′), (g′″), (i″′) and (j″′).

In certain other preferred aspects, the compounds have the above-identified specific meanings (a″ (b′″), (c′″), (d″′), (e′″), (f″′), (g′″), (h′″) and (j′″).

In certain other preferred aspects, the compounds have the above-identified specific meanings (a″ (b′″), (c′″), (d″′), (e′″), (f″′), (g″′), (h″′) and (i″′).

Particularly preferred are those of the above-identified compounds in which the meaning for R₂ under item (c″′) is either H or F.

In preferred aspects of the fourth embodiment, R₁₁ and R₁₂ are selected such that the nitrogen, to which R₁₁ and R₁₂ are attached, exhibits a pKa in the range of from 6.0 to 8.5, preferably 6.2 to 7.5 and more preferably 6.4 to 7.0;

According to specific aspects of the invention, a compound in accordance with any of the above embodiments is provided, which is selected from the group consisting of

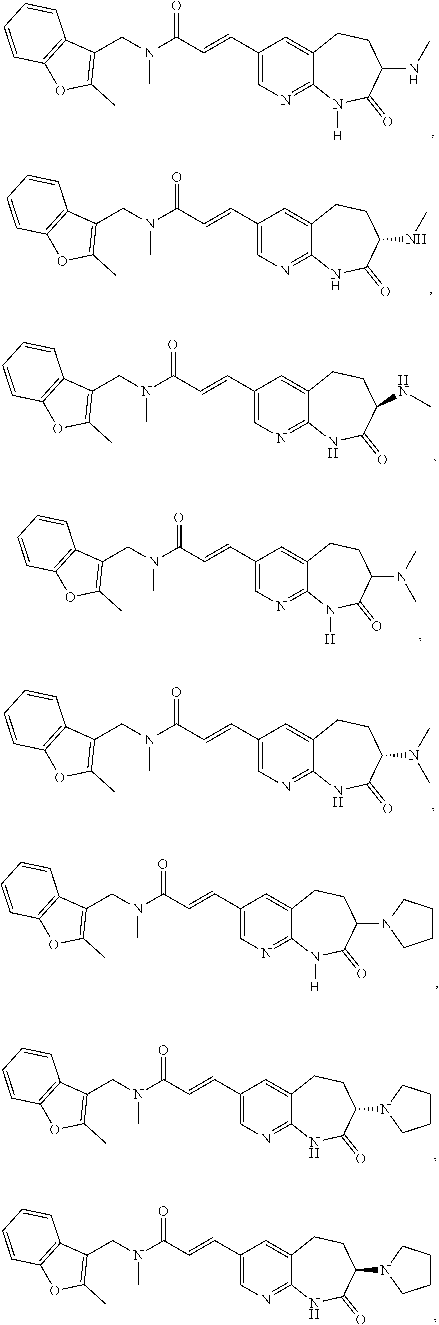

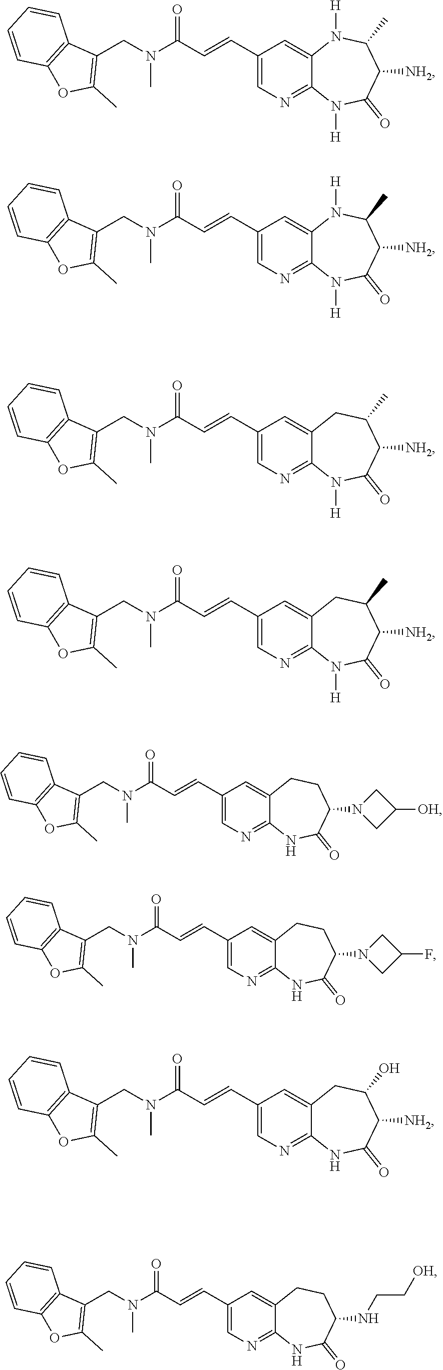

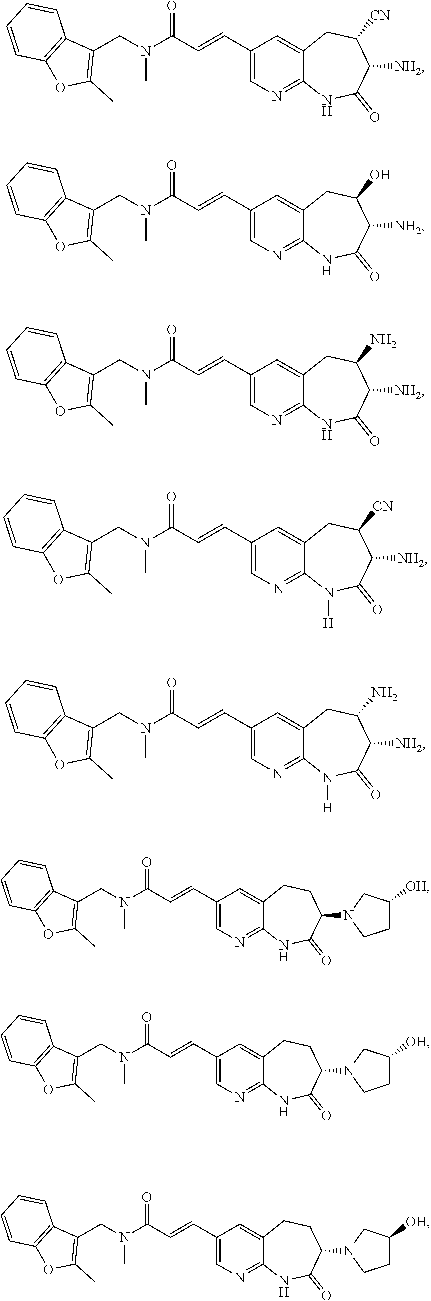

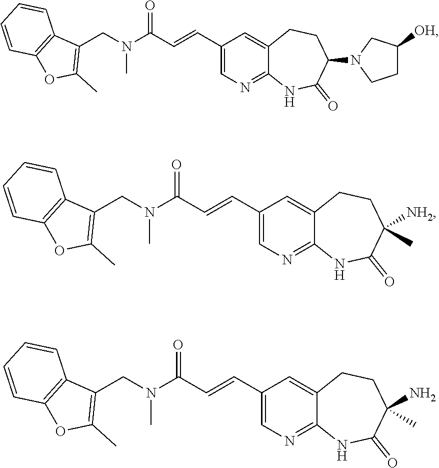 [SMILES: Cc1oc2ccccc2c1CN(C)C(=O)/C=C/c1cnc2c(c1)CC[C@@H](N1CC[C@H](O)C1)C(=O)N2 ; Cc1oc2ccccc2c1CN(C)C(=O)/C=C/c1cnc2c(c1)CC[C@](C)(N)C(=O)N2 ; Cc1oc2ccccc2c1CN(C)C(=O)/C=C/c1cnc2c(c1)CC[C@@](C)(N)C(=O)N2]

and any pharmaceutically acceptable prodrugs, salts and/or solvates thereof.

Unless expressly specified otherwise, the present disclosure contemplates all such compounds, including cis- and trans-isomers, R- and S-enantiomers, diastereomers, (d)-isomers, (I)-isomers, the racemic mixtures thereof, and other mixtures thereof, as falling within the scope of the invention. However, the carbon-carbon double bond between the pyridine ring and the amide group in the center of the molecule must be in trans configuration, as shown in the above formulae. Additional asymmetric carbon atoms may be present in a substituent such as an alkyl group. All such isomers, as well as mixtures thereof, are intended to be included in this invention.

If, for instance, a particular enantiomer of a compound disclosed herein is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Moreover, individual enantiomers and diastereomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well known methods, such as chiral-phase gas chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations. For examples, see Carreira and Kvaerno, Classics in Stereoselective Synthesis, Wiley-VCH: Weinheim, 2009.

The invention also embraces isotopically labeled compounds of the invention which are as recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P³²P, ³⁵S, ¹⁸F, and ³⁶Cl, respectively. For example, a compound of the invention may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in the e.g., Examples herein by substituting an isotopically labeled reagent for a non-isotopically labeled reagent.

### Prodrugs

Prodrugs of the present invention contain at least one prodrug moiety, i.e. a moiety that is cleaved under physiologic conditions to thereby release the active species. Such prodrug moieties may be attached to the compounds of the present invention in all positions showing sufficient reactivity. For example, with the meaning of R_{d}, R₁₃ may be a prodrug moiety or there may be a prodrug moiety such as R_{d} attached to Y if Y is N. In preferred aspects of the invention, a prodrug is thus formed by selecting a group R_{d} as R₁₃ or as the substituent attached to Y being N.

### Salts, Solvates, Polymorphs

The compounds of the present invention may be used in the free form or, alternatively, in the form of pharmaceutically acceptable salts. Acid addition salts are particularly suitable. Pharmaceutically acceptable salts that can be used in the present invention are well-known to the skilled person and are disclosed, for instance, in S. M. Berge et al., J. Pharm. Sci., 1977, 66, 1, 1-19; R. J. Bastin, et al., Org. Proc. Res. Dev., 2000, 4, 427-435; and P. H. Stahl, C. G. Wermuth, Eds. “Pharmaceutical Salts: Properties, Selection, and Use″, 2^{nd} Ed. Wiley-VCH, 2011. Particularly effective salts may be hydrochloride salts e.g. hydrochloride or dihydrochloride salts, or fluoroacetate salts e.g. trifluoroacetate salts.

The prodrugs of the present invention may also be provided in the free form or in the form of pharmaceutically acceptable salts. Suitable are pharmaceutically acceptable salts well-known to the skilled person, e.g. as described in the literature cited above.

The compounds of the invention can exist in unsolvated forms as well as in solvated form with pharmaceutically acceptable solvents such as water, ethanol, and the like, and it is intended that the invention embrace both solvated and unsolvated forms.

The compounds of the invention may exist in single or multiple crystalline forms or polymorphs. In one embodiment, the compound is amorphous. In one embodiment, the compound is a single polymorph. In another embodiment, the compound is a mixture of polymorphs. In another embodiment, the compound is in a crystalline form.

### Pharmaceutical Compositions

Compounds of the present invention may be comprised in pharmaceutical compositions. Said pharmaceutical compositions of the disclosure may be administered by various means and may take any appropriate form of formulation, depending on their intended use, as is well known in the art. For example, if compositions of the disclosure are to be administered orally, they may be formulated as tablets, capsules, granules, powders or syrups. Alternatively, compositions disclosed herein may be administered parenterally and formulated as injections/injectables (intravenous, intramuscular, intraperitoneal or subcutaneous), drop infusion preparations or suppositories. For application by the ophthalmic mucous membrane route, the compositions disclosed herein may be formulated as eye drops or eye ointments. The compositions may comprise any conventional additive, such as an excipient, a binder, a disintegrating agent, a lubricant, a corrigent, a solubilizing agent, a suspension aid, an emulsifying agent or a coating agent. Wetting agents, emulsifiers and lubricants, such as sodium lauryl sulfate and magnesium stearate, as well as coloring agents, release agents, coating agents, sweetening, flavoring and perfuming agents, preservatives and antioxidants may also be comprised in the compositions.

In the compositions of the invention, additives may serve more than one function. For example, fillers or binders may also be disintegrants, glidants, anti-adherents, lubricants, sweeteners and the like.

The compositions may be prepared by any conventional means, which may depend on the type of formulation in question e.g. tablet, injection etc. The composition may comprise any conventional excipient and/or additive e.g. one or more of those set out above.

The compositions may be formulated to be suitable for oral, nasal (e.g. by inhalation by formulating a dry powder formulation or a nebulized formulation), rectal, vaginal, aerosol and/or parenteral (e.g., by injection, for example, intravenous, intraperitoneal, intramuscular, or subcutaneous injection) administration. Said compositions may conveniently be presented in unit dosage form and may be prepared by any methods well known in the art of pharmacy. The amount of a compound disclosed herein that may be combined with an excipient e.g. carrier material to produce a single dose may vary depending upon the identity of the compound, the subject being treated, and the particular mode of administration.

As stated previously, the compositions of the invention may be prepared by any conventional means, said conventional means may depend on the desired form of the composition e.g. tablet, injection/injectable. Methods of preparing the compositions of the invention may include the step of bringing into association a composition of the disclosure with a carrier and, optionally, one or more additional additive ingredient. In general, the compositions are prepared by uniformly and intimately bringing into association compound of the invention with liquid carriers, or finely divided solid carriers, or both, and then, if necessary, shaping the product.

Composition for the invention formulated to be suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the disclosure may also be administered as a bolus, electuary, or paste.

In solid dosage forms for oral administration (capsules, tablets, pills, dragees, powders, granules and the like), the subject composition may be mixed with one or more pharmaceutically acceptable excipients selected from: (1) fillers or extenders, such as starches, dextrose, lactose, sucrose, glucose, mannitol, and/or silicic acid; (2) binders, such as, for example, celluloses (e.g., microcrystalline cellulose, methyl cellulose, hydroxypropylmethyl cellulose (HPMC) and carboxymethylcellulose), alginates, gelatin, polyvinyl pyrrolidone, sucrose and/or acacia; (3) humectants, such as glycerol; (4) disintegrating agents, such as croscarmellose sodium, sodium carboxymethyl starch (sodium starch glycolate), crosslinked polyvinylpyrrolidone (crospovidone), gellan gum, xanthan gum, agar-agar, calcium carbonate, potato or tapioca starch, alginic acid and sodium alginate, certain silicates and especially calcium silicate, and sodium carbonate; (5) dissolution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; (10) coloring agents; (11) complexing agents such as cyclodextrins and modified cyclodextrins including preferably (2-hydroxypropyl)-β-cyclodextrin and sulfobutylether-β-cyclodextrin; (12) matrix-forming polymeric excipients such as polyvinyl pyrrolidone (PVP), e.g. PVP K30, acrylic polymers and co-polymers such as the different grades of Eudragit and preferably Eudragit L100, hydroxypropylmethyl cellulose acetate succinate (HPMCAS), other copolymers such as polyethylene glycol-based copolymers like Soluplus; and (13) carriers, such as sodium citrate or dicalcium phosphate. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like. The disclosed excipients may serve more than one function. For example, fillers or binders may also be disintegrants, glidants, anti-adherents, lubricants, sweeteners and the like. It is possible in accordance with the present invention to use two or more excipients, wherein said two or more excipients may belong to the same and/or different categories. There is no restriction in this respect.

Composition of the invention formulated for parenteral administration, including intravenous, intramuscular, intraperitoneal or subcutaneous administration, may be provided in solid form in vials such that they can be diluted in a suitable solvent (e.g. oil, or water, aqueous NaCl solution e.g. 0.9 wt. % NaCl solution, aqueous glucose solution, dextrose solution). The solid form may comprise, a compound of formula (I) mixed with one or more of an excipient and/or an additional ingredient for example a buffer such as sodium citrate, a solubilizer (co-solvent) e.g. ethanol, a complexing agent (such as cyclodextrins and modified cyclodextrins including preferably (2-hydroxypropyl)-β-cyclodextrin and sulfobutylether-β-cyclodextrin), a stabilizer e.g. cellulose, 2-hydroxypropyl ether, Polyethylene Glycol 4000 crosslinked polyvinylpyrrolidone (crospovidone) and/or polyethylene glycols, an osmotic agent e.g. glucose or sodium chloride, a surfactant e.g. Polyoxyethylene 20 sorbitan monooleate, polyoxyl castor oil and/or sodium lauryl sulfate, a preservative or bacteriostat e.g. sodium citrate, benzyl alcohol and/or viscosity modifier as benzyl alcohol or carboxymethylcellulose. Other pharmaceutically acceptable excipients may also be suitable for inclusion in said solid forms e.g. one or more of the pharmaceutically acceptable excipients set out hereinabove as being suitable for inclusion in compositions formulated for oral administration. It is well within the purview of the skilled person to select appropriate excipients depending on the desired properties of the solid form. A composition formulated for parenteral administration may also be provided in liquid form, e.g. in an infusion bag or in a prefilled syringe. In this case, the same components as listed above may be present in the liquid formulation. The liquid formulation may be an aqueous formulation, aqueous NaCl solution, e.g. 0.9 wt. % NaCl solution, aqueous glucose solution, or dextrose solution, the liquid formulation may also be an oil formulation e.g. a stabilized oil in water emulsion, comprising medium chain triglycerides and long chain triglycerides, stabilized by phospholipids.

Further parenteral administration types are also conceivable, including in particular medical or antibiotic implants comprising a compound of the present invention in the medical or antibiotic implant or in a coating on the medical or antibiotic implant.

The term “medical implant” as used herein refers to any indwelling (placed inside the body of a patient) medical device intended to replace, support or enhance a biological structure. Medical implants may be placed permanently, e.g. a stent or prosthetic joint, alternatively they can be placed on a temporary basis and removed when they are no longer needed e.g. a chemotherapy port or orthopedic screw.

The term “antibiotic implant” as used herein refers to any indwelling (placed inside the body of a patient) medical device, wherein said medical device is implanted in a patient with the primary intention of treating or preventing infection e.g. bacterial infection through the delivery of antibiotics. Antibiotic implants may be placed permanently, alternatively they can be placed on a temporary basis and removed when they are no longer needed e.g. when an infection has been eradicated, or they may simply dissolve over time in the body.

The compound of the present invention may also be applied to medical instruments e.g. surgical instruments or sutures. This may prevent bacterial growth on said medical instrument. Said medical instrument may also deliver the antibiotic(s) at a surgical site, or a wound site e.g. in the case of a suture.

The term medical instrument as used herein refers to any tool used in a medical setting for the diagnosis or treatment of patients e.g. surgical tools such as scalpels and forceps, scissors and sutures. The term “medical instrument” as used herein encompasses dental instruments.

Common excipients, especially for compositions formulated for oral or IV administration include, Stabilising agents

A stabilizing agent may be advantageously used to improve the formulation's physico-chemical stability. There is no particular limitation on the stabilizing agent that can be employed in the present invention.

The use of endotoxin controlled PVP and/or Polyvinylpyrrolidone may be preferred as a stabilizing agent for a composition formulated for parenteral administration.

The stabilizing agent may be present in a relative amount of from 0.01 wt % to 20 wt %, preferably from 0.1 wt % to 2 wt % and more preferably 0.1 wt % to 1 wt %. The wt %-indications herein and in the subsequent sections are based on the total weight of the pharmaceutical composition being 100 wt %.

### Buffers

A buffer may be advantageously used to control the pH solution of a parenteral formulation There is no particular limitation on the buffer that can be employed in the present invention.

The employed buffer may depend on the physico chemical characteristics of a compound of the invention e.g. stability and solubility, the capacity of the buffer, and the desired pH. Phosphate, citrate, tris, succinate, and/or histidine buffer can for example be used.

The buffer may be present in a relative amount of from 0.01 wt % to 5 wt %, preferably from 0.01 wt % to 5 wt % and more preferably 0.01 wt % to 3 wt %.

### Solubilizer (Co-Solvent)

A solubilizer (co-solvent) may be advantageously used to improve the solubility of a compound of the invention. There is no particular limitation on the solubilizer (co-solvent) that can be employed in the present invention.

The use of a biocompatible co-solvent may be preferred, e.g. Polyoxyethylene 300 or 400, ethanol, propylene glycol and/or glycerin.

The co-solvent may be present in a relative amount of from 1 wt % to 60 wt %, preferably from 1 wt % to 30 wt % and more preferably 1 wt % to 15 wt %.

### Osmotic Agents

An osmotic agent may be advantageously used to reach solution's isotonicity. There is no particular limitation on the osmotic agent that can be employed in the present invention.

The use of glucose and/or sodium chloride may be preferred.

The osmotic agent may be present in a relative amount of from 0.01 wt % to 20 wt %, preferably from 0.1 wt % to 5 wt % and more preferably 0.09 wt % to 5 wt %.

### Preservatives

A preservative may be advantageously used to protect the compound from physico-chemical degradation, like oxidation, light, temperature. There is no particular limitation on the preservative that can be employed in the present invention.

The use of sodium bisulfite, sodium metabisulfite, ascorbate, sodium sulfite, and/or thioglycerol may be preferred.

The preservative may be present in a relative amount of from 0.01 wt % to 3 wt %, preferably from 0.01 wt % to 2 wt % and more preferably 0.01 wt % to 0.01 wt %.

### Binders

A binder may be advantageously used for increasing the particle size of active ingredient alone or with excipients and improve its handling properties. There is no particular limitation on the binder material that can be employed in the present invention.

Suitable binder materials include povidone (polyvinylpyrrolidone), copovidone (Poly(1-vinylpyrrolidone-co-vinyl acetate)), maltodextrin, poloxamer (a block copolymer with a first poly(ethylene oxide) block, a second and central poly(propylene oxide) block and a third poly(ethylene oxide) block), polyethylene glycol, polyethylene oxide, magnesium aluminosilicate, gelatin, acacia, alginic acid, carbomer (e.g. carbopol), dextrin, dextrates (a purified mixture of saccharides developed from the controlled enzymatic hydrolysis of starch), guar gum, hydrogenated vegetable oil, liquid glucose, wax, starch (pregelatinized and plain), sodium alginate and mixtures thereof.

The use of povidone and/or copovidone may be preferred.

The binder may be present in a relative amount of from 0.5 wt % to 15 wt %, preferably from 1 wt % to 12 wt % and more preferably 4 wt % to 10 wt %.

### Diluents

A diluent may be advantageously used for increasing the bulk of the pharmaceutical composition and for facilitating handling of the composition. There is no particular limitation on the diluent material that can be employed in the present invention.

Suitable diluent materials include mannitol, isomalt, histidine, lactose (including anhydrous or monohydrate forms), calcium phosphate (including dibasic and tribasic calcium phosphate), calcium carbonate, calcium sulfate, sucrose, fructose, maltose, xylitol, sorbitol, maltitol, aluminium silicate, dextrose, starch (pregelatinized or plain), glucose, dextrates (a purified mixture of saccharides developed from the controlled enzymatic hydrolysis of starch), magnesium carbonate, and mixtures thereof.

The use of mannitol, xylitol, sorbitol, isomalt and/or histidine may be preferred. Mannitol may be particularly preferred.

The diluent may be present in a relative amount that is not particularly restricted. Suitable amounts may range from 2 wt % to 85 wt %, preferably from 8 wt % to 80 wt % and more preferably 10 wt % to 50 wt %.

### Surfactant

A surfactant may advantageously be used for assisting wettability of the tablet and of the active ingredient. The surfactant is an optional but preferred component. There is no particular limitation on the surfactant material that can be employed in the present invention

Suitable surfactant materials include sodium lauryl sulfate, poloxamer, sodium docusate, sorbitan esters, polyethylene oxide, polysorbate 20, polysorbate 40, polysorbate 60, polysorbate 80 (ethoxylated sorbitan esterified with fatty acids wherein the number indicates the number of repeating units of polyethylene glycol), and mixtures thereof.

The use of sodium lauryl sulfate may be preferred.

The surfactant may be present in a relative amount that is not particularly restricted. Suitable amounts may range from 0 wt % or more to 7 wt %, preferably from 0.1 wt % to 6.5 wt % and more preferably 1 wt % to 6 wt %.

### Disintegrant

A disintegrant may be used for accelerating disintegration of the pharmaceutical composition to thereby assist in dissolution and uptake of the active ingredient. There is no particular limitation on the disintegrant material that can be employed in the present invention.

Suitable disintegrant materials include crosslinked polyvinylpyrrolidone (crospovidone), sodium carboxymethyl starch (sodium starch glycolate), croscarmellose sodium, gellan gum, xanthan gum, magnesium aluminosilicate, sodium alginate, pregelatinized starch, alginic acid, guar gum, homo- and copolymers of (meth)acrylic acid and salts thereof such as polacrillin potassium, and mixtures thereof.

The use of crospovidone may be preferred

The disintegrant may be present in a relative amount that is not particularly restricted. Suitable amounts may range from 0 wt % or more to 20 wt %, preferably from 1 wt % to 15 wt % and more preferably 2 wt % to 10 wt %.

### Glidant

A glidant may be advantageously used for improving flowability of the pharmaceutical composition to thereby improve its handling properties. The glidant is an optional but preferred component. There is no particular limitation on the glidant material that can be employed in the present invention.

Suitable glidant materials include colloidal silica dioxide, magnesium oxide, magnesium silicate, tribasic calcium phosphate, and mixtures thereof.

The use of colloidal silica dioxide may be preferred.

The glidant may be present in a relative amount that is not particularly restricted. Suitable amounts may range from 0 wt % or more to 5 wt %, preferably from 0.1 wt % to 4 wt % and more preferably 0.2 wt % to 1 wt %.

### Lubricant

A lubricant may be advantageously used to facilitate tableting, in particular by preventing sticking of the tablets to the tablet punch. The lubricant is an optional but preferred component. There is no particular limitation on the lubricant material that can be employed in the present invention.

Suitable lubricant materials include magnesium stearate, sodium stearyl fumarate, talc, stearic acid, leucine, poloxamer, polyethylene glycol, glyceryl behenate, glycerin monostearate, magnesium lauryl sulfate, sucrose esters of fatty acids, calcium stearate, aluminum stearate, hydrogenated castor oil, hydrogenated vegetable oil, mineral oil, sodium benzoate, zinc stearate, palmitic acid, carnauba wax, sodium lauryl sulfate, polyoxyethylene monostearates, calcium silicate, and mixtures thereof.

The use of a lubricant selected from magnesium stearate and sodium stearyl fumarate, and combinations thereof may be preferred.

The lubricant may be present in a relative amount that is not particularly restricted. Suitable amounts may range from 0 wt % or more to 7 wt %, preferably from 0.1 wt % to 4 wt % and more preferably 0.5 wt % to 3.5 wt %.

### Matrix Forming Polymers and Copolymers

A matrix forming polymer or copolymer may be used as an optional but preferred component. Suitable matrix-forming polymers and copolymers include polyvinyl pyrrolidine (PVP), acrylic polymers and co-polymers such as the different grades of Eudragit, hydroxypropylmethyl cellulose acetate succinate (HPMCAS), as well as other copolymers such as polyethylene glycol-based copolymers like Soluplus.

Preferred matrix-forming polymers and copolymers may be HPMC AS and Soluplus.

The matrix-forming polymers and copolymers may be present in a relative amount that is not particularly restricted. Suitable amounts may range from 0.1 g to 10 g or 0.1 wt % to 10 wt %, preferably from 0.2 g to 5 g or 0.2 wt % to 5 wt %, and more preferably from 0.3 g to 4 g or 0.3 wt % to 4 wt %.

### Complexing Agents

A complexing agent may be used as an optional but preferred component.

Suitable complexing agents include cyclodextrins and modified cyclodextrins.

Preferred complexing agents include (2-hydroxypropyl)-β-cyclodextrin and sulfobutylether-β-cyclodextrin.

The complexing agents may be present in a relative amount that is not particularly restricted. Suitable amounts may range from 0.1 g to 24 g or 0.1 wt % to 40 wt % or 30 wt % or 24 wt %, preferably from 0.1 g to 10 g or 0.1 wt % to 10 wt %, and more preferably from 0.1 g to 5 g or 0.1 wt % to 6 wt % or 5 wt %.

### Other Types of Excipients

The composition of the present invention may contain further excipients that are commonly used in the art.

Such further excipients may include release rate modifiers, plasticizer, film forming agent, colorant, anti-tacking agent and/or pigment for coating the compositions of the present invention. Further types of excipients, which may be present, include flavoring agents, sweeteners, antioxidants, absorption accelerators and/or bulking agents. Relative amounts of such excipients are not particularly limited.

They may be determined by the skilled person based on common general knowledge and routine procedures.

Film forming agents are advantageously used for providing a tablet of the invention with a coherent coating. Suitable film forming agents include isomalt, polyvinyl alcohol, polyethylene glycol, maltodextrin, sucrose, xylitol, maltitol, enteric coating agents such as materials selected from the group consisting of methyl acrylate-methacrylic acid copolymers, polyvinyl acetate phthalate (PVAP), methyl methacrylate-methacrylic acid copolymers, shellac, sodium alginate and zein.

Suitable plasticizers include sorbitol, triacetin, poloxamer, polyethylene glycol, glycerin, propylene glycol, polyethylene glycol monomethyl ether, acetyl tributyl citrate, acetyl triethyl citrate, castor oil, glyceryl monostearate, diacetylated monoglyerides, dibutyl sebacate, diethyl phthalate, triethyl citrate, and tributyl citrate.

For each of the above-mentioned categories of excipients it is possible to use only a single substance or a combination of two or more substances belonging to the same category. Of course, it is not necessary that members of each and every category are present.

The compositions of the invention may include the compounds disclosed herein in the form of particles of amorphous substance or in any crystalline form. The particle size is not particularly limited. For instance, compositions may include micronized crystals of the disclosed compounds. Micronization may be performed on crystals of the compounds alone, or on a mixture of crystals and a part or whole of pharmaceutical excipients or carriers. Mean particle size of micronized crystals of a disclosed compound may be for example about 5 to about 200 microns, or about 10 to about 110 microns. The compounds of the invention may also be present in the form of a molecular dispersion within a polymeric matrix. In yet another embodiment, the compounds of the invention may be complexed with suitable complexing agents such as cyclodextrins.

A tablet may be made by compression or molding, optionally with one or more accessory ingredients. Compressed tablets may be prepared using binder (for example, gelatin, microcrystalline cellulose, or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art. The disclosed excipients may serve more than one function. For example, fillers or binders may also be disintegrants, glidants, anti-adherents, lubricants, sweeteners and the like.

It will be appreciated that a disclosed composition may include lyophilized or freeze-dried compounds disclosed herein. For example, disclosed herein are compositions that comprise disclosed compounds in crystalline and/or amorphous powder forms. Such forms may be reconstituted for use as e.g., an aqueous composition.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Compositions formulated for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Compositions formulated into forms which are suitable for vaginal administration also include pessaries, tampons, creams, gels, pastes, foams or spray formulations containing such carriers as are known in the art to be appropriate.

Dosage forms for transdermal administration of a subject composition includes powders, sprays, ointments, pastes, creams, lotions, gels, solutions, and patches. The compound of the invention may be mixed under sterile conditions with a pharmaceutically acceptable carrier, and with any preservatives, buffers, or propellants that may be required.

The ointments, pastes, creams and gels, drops, may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the disclosure may alternatively be formulated into a form suitable for administration by aerosol. This may be accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions.

Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

It should be noted that excipients given as examples may have more than one function. For example, fillers or binders can also be disintegrants, glidants, anti-adherents, lubricants, sweeteners and the like. In one embodiment, fulfillment of amount indications specified hereinabove for different types of excipients is to be assessed for each type of excipient taking into account the total amount of all excipients having the specified function.

Pharmaceutical compositions of this disclosure suitable for parenteral administration comprise a compound of the invention in combination with one or more pharmaceutically-acceptable sterile isotonic aqueous or non-aqueous solutions, dispersions, suspensions or emulsions, or sterile powders. Said compositions may may be reconstituted into sterile injectable solutions or dispersions just prior to use. Said compositions may contain one or more excipients as set out hereinabove e.g. antioxidants, buffers, bacteriostats, solutes which render the formulation isotonic with the blood of the intended recipient, or suspending or thickening agents. For example, provided herein is an aqueous composition that includes a disclosed compound, and may further include for example, dextrose (e.g., about 1 to about 10 weight percent dextrose, or about 5 weight percent dextrose in water (D5W).

Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of surfactants, such as lecithin, by the maintenance of the required particle size in the case of dispersions, and by the use of surfactants.

It will be appreciated that contemplated compositions and formulation forms, such as oral formulations (e.g. a pill or tablet) and parenteral formulations e.g. solutions for IV infusion, may be formulated as controlled release formulation, e.g., an immediate release formulation, a delayed release formulation, or a combination thereof.

In certain embodiments, the subject compounds and compositions may be formulated as a tablet, pill, capsule or other appropriate ingestible formulation (collectively hereinafter “tablet”) or an aqueous or non-aqueous solutions, dispersions, suspensions or emulsions for parenteral administration. The compositions of the present disclosure may be formulated such that the resulting amount of antibacterial agent i.e. compounds of the invention provided/administered to a patient (human or non-human mammal), would provide a therapeutically effective amount (a therapeutic dose). Said therapeutically effective amount may be split across dosage units e.g. multiple i.v. administrations/day for example for 3 days to 5 weeks e.g. 7 days to 2 weeks. Said therapeutically effective amount may be an amount at which at least 50% e.g. at least 60, 70, 80, 90, 95% or 100% of individuals exhibit a statistically significant reduction in infection. Said amount should also take into consideration the toxicity of said antibacterial agent(s). The therapeutically effective amount may vary depending on size, weight, age, condition and type of subject, as well as on the infection being treated and the type of formulation e.g. tablet and/or mode of administration e.g. oral or parenteral e.g. subcutaneous, intramuscular or intravenous injection. It is well within the purview of the skilled person to determine such a therapeutically effective amount employing standard drug development techniques and methodology e.g. in-vitro and/or in-vivo experiments e.g. to determine Probability target attainment (PTA), and and/or through conducting dosage determining clinical trials and toxicity/maximum tolerated dose/safety studies e.g. in animals and/or humans.

### Unit Dosages

If treatment of the patient by the pharmaceutical compositions of the present invention is by means of oral administration, a single unit dose of the pharmaceutical composition of the present invention is typically administered one, two or three times a day. The daily dosage (total dosage administered in one day) is determined by the physician in accordance with the above guidance taking the type and severity of the infection, gender, weight, age and general condition of the patient into account. Preferred oral daily dosages may range from 40 to 5000 mg e.g. 40 to 3000 mg, preferably 40 mg to 2000 mg e.g. 100 to 2000 mg. The daily dosage may vary depending on the intended frequency of administration e.g. daily, once per week.

In case of parenteral administration (for instance in intravenous (i.v.) or intramuscular (i.m.) or intraperitoneal (i.p) or subcutaneous administration), the pharmaceutical compositions of the present invention may be administered two, three or even more times a day. Preferred daily dosages are in the range of from 40 to 5000 mg, typical unit dosages may be from 40 to 3000 mg and preferably 100 to 1000 mg. The upper limits of the specified ranges are subject to their feasibility. For instance, in case of i.m. or subcutaneous administration, it may happen that the maximum dose that can be administered in a single shot is restricted due to low solubility and correspondingly increased volume of the drug solution. In such a case, the maximum unit dosages are limited by the maximum tolerated dose.

### Drug Combinations

Compositions are also contemplated herein that include one or more of the disclosed compounds with a second component. Second components in such compositions of the present disclosure may be another antibiotic agent e.g. a Fabl inhibitor, other than a compound disclosed herein. Other additional components may also be present, including other Fabl inhibitors or other antibiotic agents. The contemplated methods of treatment disclosed herein, in some embodiments, may further comprise administering another agent such as another antibiotic agent (other than a compound disclosed herein). For example, a method of treating a bacterial infection is provided that comprises administering a disclosed compound and further comprises administering another antibiotic agent or antibacterial agent. The compound disclosed herein and the second component may be part of the same dosage form or may be formulated in two separate dosage forms. If they are formulated in two separate dosage forms, the dosage form with the second component may be administered at the same time, before or after the dosage form with the compound disclosed herein.

### Medical Indications

The compounds and compositions of the present invention may be used for treating bacterial infections in a patient. They may, in particular, be suitable for the treatment of bacterial infections involving one or more of the following bacteria: S. aureus, E. coli, Klebsiella pneumoniae and/or A. baumannii. Such infections include, but are not limited to, wound infections e.g. infections of burn wounds or surgical sites, skin and soft tissue infections such as bacterial folliculitis, impetigo e.g. localised impetigo, cellulitis, boils, feruncles, carbuncles, abscesses, dermatitis e.g. eczema; bacteraemia and sepsis, meningitis, intra-abdominal infection, pleuropulmonary infection and pneumonia including hospital acquired pneumonia, nosocomial pneumonia, and ventilator associated pneumonia; infective endocarditis; diarrhea and food poisoning e.g. by S. aureus or E. coli; urinary tract infections including complicated urinary tract infections, thrombophlebitis when caused by bacteria, osteoarticular infections such as septic arthritis, diabetic foot, bone and joint infections and prosthetic joint infections, medical device/implant related infections, infections of the oral cavity such as buccal ulcers e.g. periodontal abscess, dental infection e.g. odontogenic infection, and gingivitis; ophthalmic infections e.g. corneal ulcers; colonisation of the nasal passages by S. aureus.

In particular the compounds and compositions of the invention may be effective in the treatment of a bacterial infection associated with A. baumannii wherein said infection may be pneumonia and most preferably nosocomial pneumonia or ventilator associated pneumonia.

### Administration Types

As previously set out, the compounds and compostions of the present invention may be administered to the patient by intravenous, intramuscular, intraperitoneal or subcutaneous administration or, alternatively, by oral administration. To increase solubility and or bioavailability, the compounds may advantageously be administered in the form of prodrugs or salt form. Further administration forms are also conceivable, for instance by implantation (e.g. as part of a medical implant), by inhalation.

### Dosages

The dosage of any disclosed compound or composition will vary depending on the symptoms, age and body weight of the patient, the nature and severity of the disorder to be treated or prevented, the route of administration, and the form of the subject composition. Any of the subject compositions may be administered in a single dose or in divided doses. Dosages for the compositions may be readily determined by techniques known to those of skill in the art or as taught herein.

In certain embodiments, the dosage of the subject compounds will generally be in the range of about 0.01 ng to about 10 g per kg body weight, specifically in the range of about 1 ng to about 0.1 g per kg, and more specifically in the range of about 1 mg to 0.1 g per kg.

An effective dose or amount, and any possible effects on the timing of administration of the composition, may need to be identified for any particular composition of the disclosure. This may be accomplished by routine experiments, using one or more groups of animals (preferably at least 2 to 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including composition, amounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The use of the subject drug combinations may reduce the required dosage for any individual agent contained in the compositions because the onset and duration of effect of the different agents may be complimentary.

Toxicity and therapeutic efficacy of subject compositions may be determined by standard pharmaceutical procedures in cell cultures or experimental animals.

The data obtained from the cell culture assays and animal studies may be used in formulating a range of dosage for use in humans. The dosage of any subject composition lies preferably within a range of circulating concentrations that give rise to a statistically significant reduction in infection in at least 50% e.g. at least 60, 70, 80, 90, 95% or 100% of individuals with little or no toxicity. The dosage may vary within this range depending upon the dosage form employed and the route of administration utilized. For compositions of the disclosure, the therapeutically effective dose may be estimated initially from cell culture assays.

### Administration Frequency

The compounds and compositions disclosed herein may be administered in any appropriate frequency. Said frequency may depend on the subject being treated and on the severity and type of the infection. Administrations may for example be once or multiple times a day. The number of administrations may also depend on the form of the composition and on the subject and medical condition e.g. bacterial infection, being treated.

### Duration of Treatment

The compounds and compositions disclosed herein may be administered for an unlimited period of time. It is advantageous that they are administered for a period of time to eradicate the bacterial infection completely or at least to such an extent that the patient's immune system can cope with any remaining pathologic bacteria. Typical durations of administration may be from 3 days to 7 weeks, e.g. from 1 to 5 weeks, e.g. 7 days to 2 weeks. However, longer treatment durations may be necessary for some infections e.g. bone infections.

### Methods of Treatment

The compounds and compositions disclosed herein may be used in a method of therapy. In particular the compounds and compositions disclosed herein may be used in a method of treating a bacterial infection, comprising administering to a patient in need thereof a disclosed compound of the invention or a pharmaceutical composition comprising a disclosed compound of the invention. The bacterial infection may be an infection by S. aureus, E. coli, Klebsiella pneumoniae and/or A. baumannii.

The compounds of the invention may also be used in the manufacture of a pharmaceutical composition for use in therapy and in particular in the treatment of a bacterial infection in a patient in need thereof, wherein said bacterial infection may be by S. aureus, E. coli, Klebsiella pneumoniae and/or A. baumannii.

A further embodiment relates to a method of treating a bacterial infection, such as an S. aureus, E. coli, Klebsiella pneumoniae and/or A. baumannii bacterial infection, in a patient in need thereof comprising administering a compound or composition of the invention.

### Manufacture of the Compounds of the Invention

The compounds of the present invention can be prepared using established organic chemistry synthetic methods and procedures and/or information described hereinbelow. Starting materials may either be purchased (if commercially available) or synthesized using established organic chemistry synthetic methods and procedures and/or information described hereinbelow.

Compounds disclosed herein may be prepared by means of the following method, which comprises the step of coupling a precursor compound of formula M1 or M1′

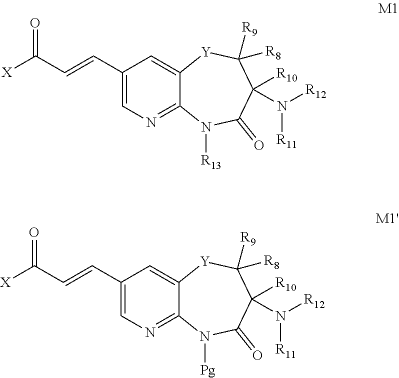

wherein X represents a leaving group, R₁₃ is as defined herein with the exception that if R₁₃ is —PO₃Rₑ₂ or -CH2-OPO₃Rₑ₂ each Rₑ is a Pg group such as TMSCH₂CH₂ or CNCH₂CH₂, and Pg in M1′ represents a protective group such as a BOC group, and wherein R₁₁ and R₁₂ may be a group as defined in any of the claims or items disclosed herein with respect to R₁₁ and R₁₂ or may be such a defined group that also comprises a protective group, which is preferably selected from the Boc group, PMB group, and DMB group, with an amine compound of formula M2b:

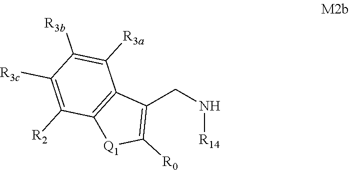

wherein R₀ to R₁₂, R₁₄, Y and Q₁ have the same meanings as specified for formula I. The leaving group X may be a hydroxyl group, a tosylate group, a triflate group, a mesylate group, iodide, bromide, chloride, methoxy, ethoxy, and the like.

The coupling reaction is preferably carried out in a solvent and in the presence of a coupling agent and a base. The solvent is preferably selected from DMF, 2-Me-THF, DCM, EtOAc, DMC, CPME (preferably the solvent is DMF if the leaving group is a hydroxyl group). The coupling agent is preferably selected from HATU, HBTU, HCTU, TBTU, COMU, TOMBU, COMBU, PyBOP, T₃P, DIC-HOBt, DCC, CDI, EDC, EDC-HOBt (preferably the coupling agent is HATU or T₃P if the leaving group is a hydroxyl group). The reaction is typically carried out in the presence of a base. The base is preferably selected from DIPEA, TEA, pyridine or DMAP (preferably TEA is used as a base when T₃P is used as a coupling agent). The protective group(s) of M1′ may preferably be removed directly after the coupling reaction. While any protecting groups on R₁₃ and/or R₁₁ and/or R₁₂ are preferably removed as the final step.

An example reaction sequence is illustrated by the following Scheme 1. An analogous reaction scheme applies for the protected precursor M1′. Of course, this analogous scheme needs to be supplemented by a preceding protection reaction and a subsequent deprotection reaction.

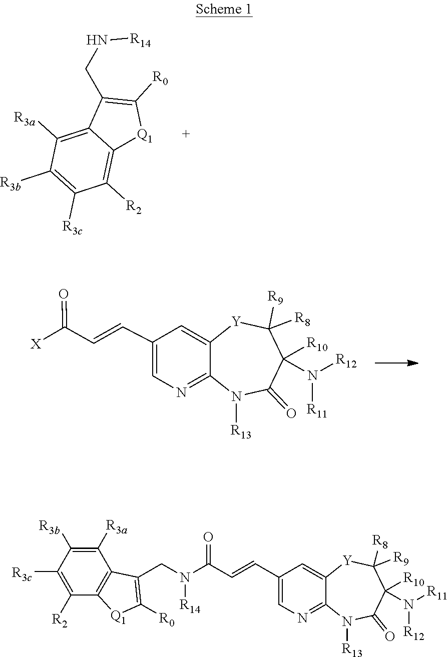

### Manufacture of Right-Hand Side Precursor

The precursor compound of formula M1′ can be manufactured by reacting a compound of formula M3/M3′, wherein Pg in M1′ is a suitable protecting group such as Boc and wherein R₁₃ is as defined herein with the exception that if R₁₃ is —PO₃Rₑ₂ or —CH₂—OPO₃Rₑ₂ each Rₑ is a Pg group such as TMSCH₂CH₂ or CNCH₂CH₂, and R₁₁ and or R₁₂ in addition of being as defined herein may also comprise a protective group, which is preferably selected from the Boc group, PMB group, and DMB group,

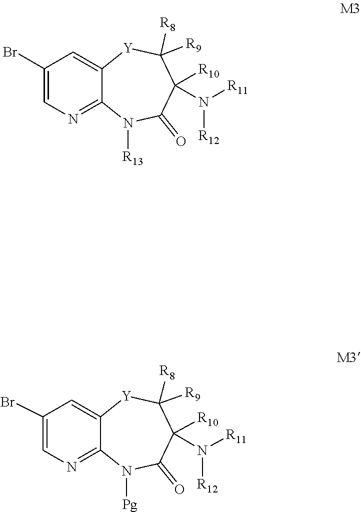

with a carboxyl-protected acrylic acid, such as a C₁₋₄-alkyl ester (preferably tert-butyl, ethyl or methyl ester) of acrylic acid. This coupling reaction is carried out under Heck coupling conditions and preferably in the presence of a Pd(II)-salt such as Pd(OAc)₂ and a phosphine ligand such as Xantphos, XPhos, or tri-(o-tolyl)phosphine or 1,1-bis(diphenylphosphino)ferrocene (dppf). Highly Efficient Palladium catalyst —Pd-162 in the presence of Cy₂NMe₂ and NBu₄CI can also be applied. The reaction is typically carried out in the presence of a solvent such as DMF, proprionitrile, a combination thereof, or 1,4-dioxane, and also in the presence of a base such as DIPEA. Such a reaction sequence is illustrated by the following Scheme 2.

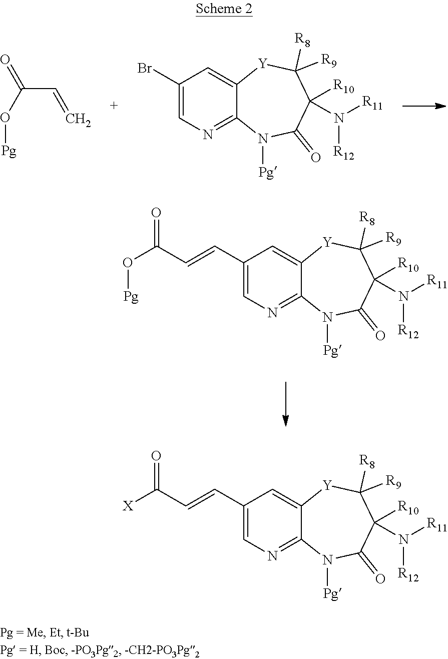

The coupling reaction is followed by deprotection of the carboxyl group and optionally introduction of a leaving group other than hydroxyl. The leaving group X may be a hydroxyl group, a tosylate group, a triflate group, a mesylate group, iodide, bromide, chloride, and the like. Pg represents a protective group suitable for the carboxyl functional group to be protected, e.g. an alkyl group (Me, Et, t-Bu) for protection of the carboxyl group. The nitrogen atom in the amide group may optionally be protected with a suitable protective group (Pg′), such as a BOC group or alternatively a protected prodrug group wherein Pg″ group is a group such as TMSCH₂CH₂ or CNCH₂CH₂, and wherein R₁₁ and R₁₂ may be a group as defined in any of the claims or items disclosed herein with respect to R₁₁ and R₁₂ or may be such a defined group that also comprises a protective group, which is preferably selected from the Boc group, PMB group, and DMB group.

The precursor compound M3 can be synthesized as shown in Schemes 3 and 4 as explained below.

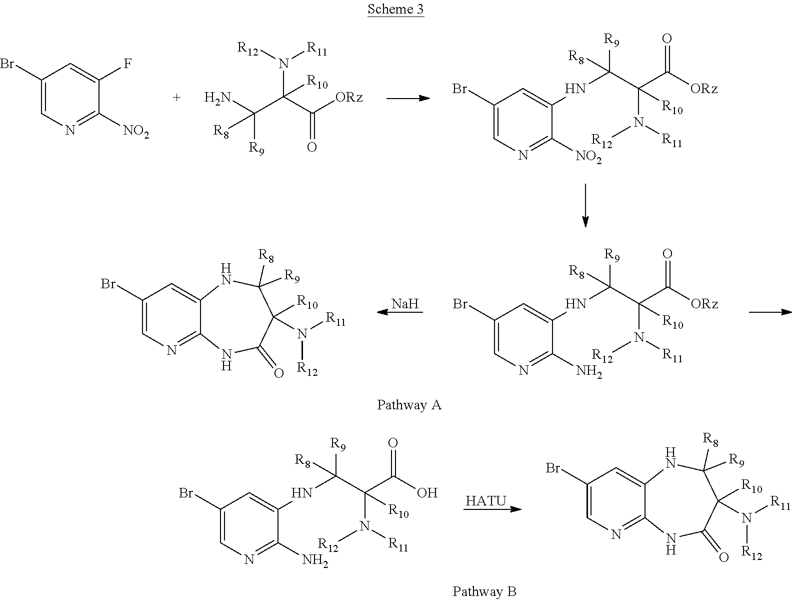

In Scheme 3 condensation of 5-bromo-3-fluoro-2-nitropyridine with appropriate acids, where R₁₁ and or R₁₂ in addition of being as defined herein may also comprise a protective group, which is preferably selected from the Boc group, PMB group, and DMB group, and where Rz=H, for instance ((S)-3-amino-2-((tert-butoxycarbonyl)amino)propanoic acid or esters in which Rz being C₁₋₄ alkyl, preferably methyl, for instance methyl 3-amino-2-((2S,6R)-2,6-dimethylmorpholino)propanoate or (2S,3R)-methyl 3-amino-2-((tert-butoxycarbonyl)amino)butanoate in THF or ACN in the presence of inorganic (K₂CO₃) or organic bases (Et₃N) leads to 3-(substituted-amino)propanoates or butanoates in good yield. The nitro group reduction at position 2 is carried out in the presence of a reducing agent such as Fe in acetic acid or mixture of water, ethanol and ammonium chloride at 80° C. The cyclization is accomplished using sodium hydride in DMF (Pathway A). This reaction sequence is illustrated by the Scheme 3. The protective group Rz is removed by basic hydrolysis using lithium hydroxide in a mixture of water and THF. The cyclization using agents such as HATU in the presence of a base like DIPEA and in a solvent such DMF (Pathway B) leads to the formation of 3-amino-8-bromo-1,2,3,5-tetrahydro-4H-pyrido[2,3-b][1,4]diazepin-4-one derivatives as shown in Scheme 3.

The precursor compound of formula M3 can be also manufactured by reaction of a compound of formula M4 as shown in Scheme 4 below.

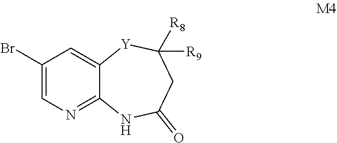

Compound of formula M4 can be synthesized as described in AFFINIUM PHARMACEUTICALS, INC. WO2007/67416, 2007, A2 which is hereby incorporated by reference.

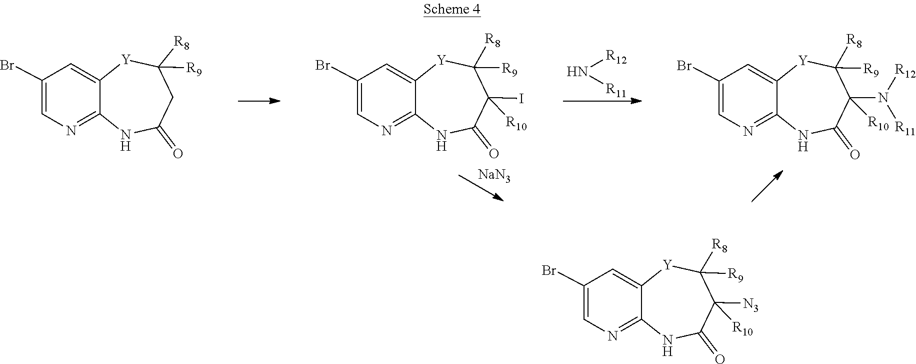

The direct iodination of 3-bromo-5,6,7,9-tetrahydro-8H-pyrido[2,3-b]azepin-8-one in the presence of TMEDA, TMSI, 12 in DCM led to the formation of expected iodide in good yield. The iodide can be easily converted into the corresponding amines (primary, secondary, tertiary and heterocyclic compounds) by its treatment with different amines HNR₁₂R₁₁ wherein R₁₂ and R₁₁ are as defined above with respect to formula I (e.g. azetidine-3-ol, morpholine, pyrrolidine and its derivatives, cyclopropanoamine, piperazine and its derivatives, 7-oxa-2-azaspiro[3.5]nonane, thiomorpholine 1,1-dioxide. etc) in acetonitrile at 50-80° C. in the presence of K₂CO₃ as a base. Alternatively, iodide reaction with sodium azide in DMF and its consecutive reduction provides the corresponding primary amines.

Manufacture of left-hand side Precursor

The left-hand side precursor M2b can be prepared by means of the reaction sequence shown in the following Scheme 5. Reduction of the protected carboxyl group to the hydroxymethyl group can be accomplished using diisobutylaluminium hydride (DIBAL-H) in THF. The subsequent oxidation to the aldehyde can be carried out using Dess-Martin periodinane in DCM. The last reaction of this sequence can be performed by first reacting with methylamine in ethanol/THF followed by reduction with sodium borohydride in ethanol/THF. If it is desired to obtain precursor M2b in protected form, the obtained product, i.e. the compound shown below but with a hydrogen in the position of Pg, may be subjected to a final step of protection of the amino group with a suitable protective group, for instance the carboxybenzyl (Cbz) group, by reaction with carboxybenzylchloride in DCM in the presence of triethylamine. This optional final protection step is also shown in Scheme 5 below.

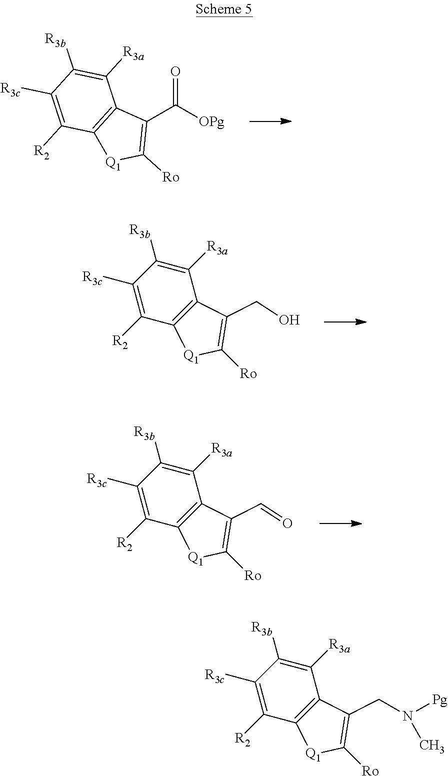

Alternatively the left-hand side precursor M2b can be prepared by means of the reaction sequence shown in scheme 5′ wherein the carboxylic acid (or its alkyl ester) is converted into its corresponding amide via amidation (e.g. by means of MeNH₂·HCl, DIPEA, EDCI-HOBt, DMF or MeNH2/EtOH, reflux), after which amide reduction (e.g. using BMS in THF or triflic anhydride/NaBH₄ in DCM) gives the corresponding amine.

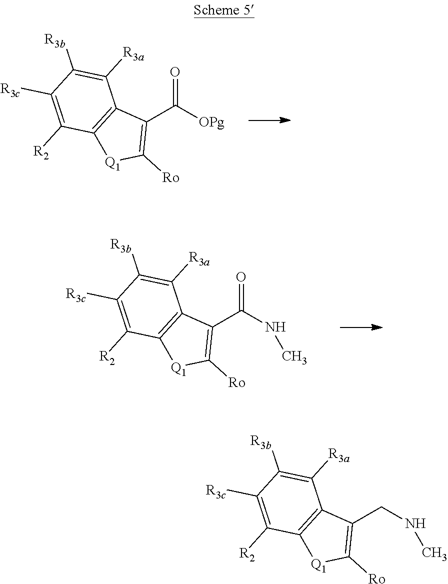

Alternatively (when Q₁ is O), the left-hand side precursor M2b can be prepared by means of one of the reaction sequences shown in Scheme 5″ wherein a carboxyphenol is substituted using ethyl-2-bromopropanoate in the presence of a base such as K₂CO₃ or NaOH in solvents such as ACN or THF, followed by decarboxylative cyclization mediated by bases such as sodium acetate in acetic anhydride to provide the benzofuran bicycle in which a carbonyl moiety is introduced at 3-position using dichloro(methoxy)methane in the presence of a Lewis acid catalyst such as tin(IV)chloride:

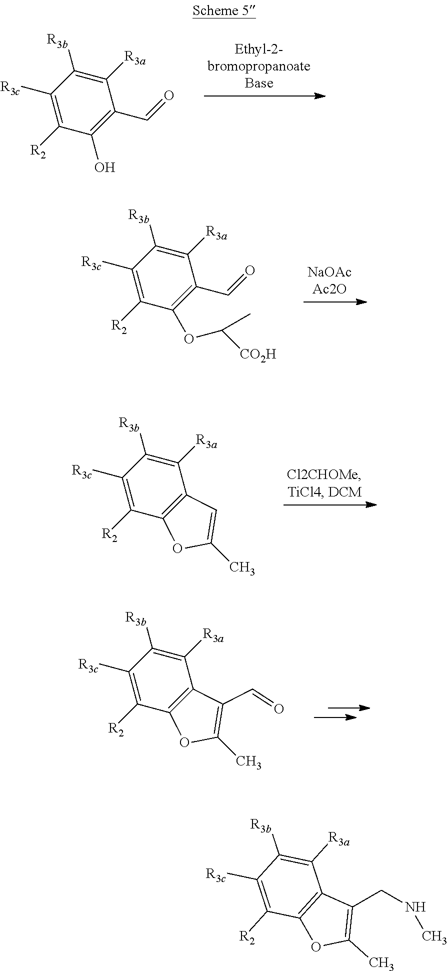

In Schemes 5, 5′, and 5″, Pg represents a protective group such as a carboxybenzyl group (BOC group, PMB group, DMB group). Q₁ has the same meaning as Q₁ in formula I (but with the restrictions to Q₁ described for Scheme 5″ above). R₂, R₃ₐ, R_{3b} and R₃c may also have the same meanings as in formula 1.

Alternatively, one or more of these groups may be a precursor group that is later converted to the desired substituent in accordance with formula 1. For instance, a Br substituent may be used as such a precursor.

When R₁₄ is different from CH₃ (R₁₄ together with R₀ of LHS form a heterocycle comprising the N to which R₁₄ is attached and having 5 to 8 ring members, wherein preferably the only heteroatom in said ring is the N to which R₁₄ is attached) the precursor M2b can be prepared by means of the reaction sequences shown in scheme 5″′ wherein an amino(thio)phenol precursor is condensed with a cyclic 1,3-dione, converted to the oxime, then submitted to a Beckmann rearrangement and having its amide reduced to the amine:

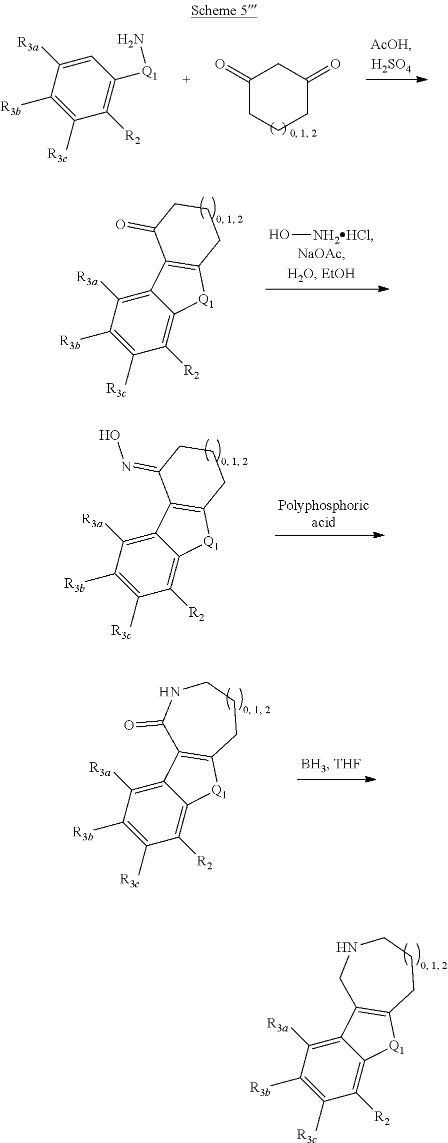

Alternative Route of Manufacture of Compounds of Invention

As an alternative to the synthetic strategy described above, the compounds of the present invention may also be prepared by coupling a compound of formula M6 or its protected form M6′

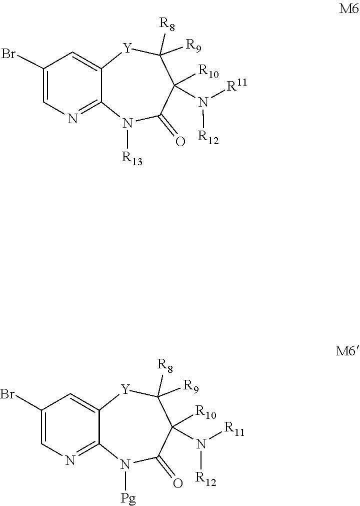

with a compound of formula M7b:

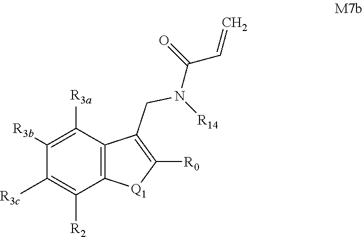

wherein Y and Q₁ have the same meaning as specified for formula (1), and all R groups (R₀ to R₁₄) have the same meanings as specified for formula 1, or may be precursors thereof e.g. Br as a precursor for other groups e.g. CN, OH, esters, etc., or R₁₁ and or R₁₂ in addition of being as defined herein may also comprise a protective group, which is preferably selected from the Boc group, PMB group, and DMB group,

This coupling may be carried out under Heck coupling conditions. Typically, it is carried out in the presence of a Pd(II) complex such as Pd-162 (i.e. [P(tBu)₃] Pd(crotyl) CI), tetrabutylammonium chloride, N-cyclohexyl-N-methylcyclohexanamine (DIPEA) and dioxane. It is also possible to use a combination of a Pd(II)-salt such as Pd(OAc)₂ with a phosphine ligand such as tri-o-tolylphosphine, a base like DIPEA and a solvent such as a mixture of DMF and propionitrile, or 1,4-dioxane. The reaction is illustrated for compounds of formula la by the following reaction scheme:

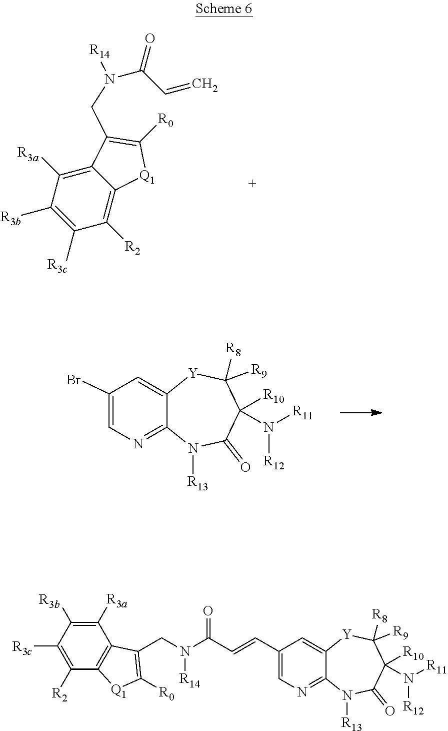

It is advantageous to use the protected precursor M6′ in this reaction sequence. In this case, the reaction sequence shown in the above scheme may be followed by a deprotection step to obtain the reaction product shown above.

Y and Q₁ have the same meanings as specified for formula I, and R₀ to R₁₂ have the same meanings as specified for formula (I) or may be precursors thereof e.g. Br as a precursor for other groups e.g. CN, OH, esters, etc., or R₁₁ and or R₁₂ in addition of being as defined herein may also comprise a protective group, which is preferably selected from the Boc group, PMB group, and DMB group, while R₁₃ is hydrogen.

The preparation of prodrugs of the compounds of the invention e.g wherein R₁₃ is —PO₃Rₑ₂ or —CH₂—OPO₃Rₑ₂, is typically accomplished by converting the respective compound of the invention with R₁₃ being hydrogen to a compound of the same structure except that R₁₃ represents a prodrug moiety that is cleavable under physiologic conditions for instance a phosphate-containing group as specified above. The prodrug moiety is preferably a methylene phosphate moiety or a phosphoramidate moiety. Such prodrug moieties and suitable reaction conditions for manufacturing methylene phosphate prodrugs are described in WO 2013/190384 A1 (methylenephosphate) and J. Med. Chem. 2000, 43, 1234-1241 (phosphoramidate).

### ABBREVIATIONS

The following abbreviations are used in the present disclosure.
CC Column chromatography
DCM Dichloromethane
N Normal
g Gram
pH Potential of Hydrogen
mol Mole
v/v Volume/volume
vol Volume
m/z Mass to charge ratio
° C. degree Celsius
TEA, Et3N Triethylamine
Et2O Diethyl ether
HPLC High performance liquid chromatography
Boc tert-butyloxycarbonyl
h hour
mL milliliter
eq. Equivalent
M Mass
Me Methyl group
MeOH Methanol
AcOH Acetic acid
THE Tetrahydrofuran
DIPEA N,N-Diisopropylethylamine
Pd(OAc)2 Palladium(II) acetate
EtOH Ethanol
DCE 1,2-Dichloroethane
EtOAc Ethyl acetate
Aq. Aqueous
RT, rt Room temperature
Rt, tret Retention time
DMF Dimethylformamide
ACN Acetonitrile
NH40Ac Ammonium acetate
TFA Trifluoroacetic acid
HOBT/HOBt 1-Hydroxybenzotriazole
TLC Thin layer chromatography
H20 Water
sat. Saturated
sol. Solution
EDCI 1-Ethyl-3-(3-dimethylaminopropyl)carbodiimide
NMR Nuclear Magnetic Resonance
s singlet
d doublet
t triplet
m multiplet
dd double of doublet
MHz Megahertz
ppm parts per million
H Proton
J Coupling constant
UPLC-MS Ultra-performance liquid chromatography-tandem mass spectrometry
DMSO Dimethyl sulfoxide
CDCl3 Deuterated chloroform
ML Mother liquor
SCX Strong Cation Exchange Chromatography
LCMS Liquid Chromatography Mass Spectrometry
HATU 1-[Bis(dimethylamino)methylene]-1H-1,2,3-triazolo[4,5-b]pyridinium 3-oxid hexafluorophosphate
HPLC High-performance liquid chromatography
HBTU (2-(1H-Benzotriazol-1-yl)-1,1,3,3-tetramethyluronium hexafluorophosphate, Hexafluorophosphate Benzotriazole Tetramethyl Uronium
Cy2NCH3, N-Cyclohexyl-N-methylcyclohexanamine
DCHMA
PMB β-Methoxybenzyl
STAB Sodium triacetoxyborohydride
DMC Dimethyl carbonate
EtOAc Ethyl acetate
HCTU O-(1H-6-Chlorobenzotriazole-1-yl)-1,1,3,3-tetramethyluronium hexafluorophosphate
TBTU 3-[Bis(dimethylamino)methyliumyl]-3H-benzotriazol-1-oxide hexafluorophosphate
COMU (1-Cyano-2-ethoxy-2-oxoethylidenaminooxy)dimethylamino-morpholino-carbenium-hexafluorophosphate
TOMBU N-{[1,3-Dimethyl-2,4,6-trioxotetrahydropyrimidin-5(6H)-ylidenaminooxy](dimethylamino)methylen}-N-methylmethanaminium hexafluorophosphate
COMBU 4-{[1,3-Dimethyl-2,4,6-trioxotetrahydropyrimidin-5(6H)ylidenaminooxy](dimethylamino)methylen}morpholin-4-ium hexafluorophosphate
PyBOP Benzotriazol-1-yloxy)tripyrrolidinophosphonium hexafluorophosphate
T3P 2,4,6-Tripropyl-1,3,5,2k5,4k5,6k5-trioxatriphosphinane 2,4,6-trioxide
DIC N,N′-Diisopropylcarbodiimide
DCC N,N′-Dicyclohexylcarbodiimide
CDI 1,1′-Carbonyldiimidazole
EDC 3-(Ethyliminomethyleneamino)-N,N-dimethylpropan-1-amine
DMAP N,N-Dimethylpyridin-4-amine
DMB 3,4-Dimethoxybenzyl
BMS Borane-dimethyl sulfide
DIAD Diisopropyl azodicarboxylate
BrettPhos 2-(Dicyclohexylphosphino)3,6-dimethoxy-2′,4′,6′-triisopropyl-1,1′-biphenyl
DMP Dess-Martin periodinane
DIBAL Diisobutylaluminum hydride
Pd-162 Tri-tert-butylphosphine(chloro)(crotyl)palladium(II)
Pd-173 Crotyl(2-dicyclohexylphosphino-2′,4′,6′-triisopropyl-3,6-dimethoxy-1,1′-biphenyl)palladium(II) triflate
Pd-175 Allyl(2-di-tert-butylphosphino-3,6-dimethoxy-2′,4′,6′-triisopropyl-1,1′-biphenyl)palladium(II) triflate
Pd₂(dba)3 Tris(dibenzylideneacetone)dipalladium(0)
NBS 1-Bromo-2,5-pyrrolidinedione; N-bromosuccinimide
pTSA 4-Methylbenzene-1-sulfonic acid
LDA Lithium diisopropylamide
BINOL 1,1′-Bi-2-naphthol
DMA N,N-Dimethylacetamide
DABCO 1,4-Diazabicyclo[2.2.2]octane
DPPF 1,1′-Bis(diphenylphosphanyl)ferrocene
Xphos 2-Dicyclohexylphosphino-2′,4′,6′-triisopropylbiphenyl
BuLi n-Butyllithium
DPPA Diphenylphosphoryl azide
mesyl Methanesulfonyl

It should be appreciated that all features of the present invention disclosed herein can be freely combined and that variations and modifications may be made without departing from the scope of the invention as defined in the claims. Where known equivalents exist to specific features, such equivalents are incorporated as if specifically referred to in this specification. Furthermore, it should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

### EXAMPLES

The following examples are in no way intended to limit the scope of the present invention, but are provided only to illustrate the inventive compounds and their preparation.

### General Procedures.

All starting materials and solvents were obtained either from commercial sources or prepared according to the literature citation. Unless otherwise stated all reactions were stirred. Organic solutions were routinely dried over anhydrous magnesium sulfate or sodium sulfate.

Column chromatography was performed on pre-packed silica (230-400 mesh, 40-63 Ipm) cartridges using the eluent indicated. SCX was purchased from Silicycle and treated with 1M hydrochloric acid prior to use. Unless stated otherwise the reaction mixture to be purified was first diluted with MeOH and made acidic with a few drops of AcOH. This solution was loaded directly onto the SCX and washed with MeOH.

The desired material was then eluted by washing with 0.7 M NH3 in MeOH.

### Analytical Methods

### Analytical UPLC/MS.

Method 1b: Waters Acquity UPLC HClass instrument with Acquity PDA detector, QDA mass detector and quaternary solvent system; PDA: 210-350 nm. Acidic methods were run using varying gradients of acetonitrile and water with 5% 2 vol % formic acid (99%) in water on the following columns: Acquity CSH C18 column (2.1×50 mm 1.7 Ipm) at 0.8 mL.

Standard method: Waters Acquity UPLC with Acquity PDA detector, SQ mass detector and quaternary solvent system; PDA: 210-400 nm. The gradient from 5-100% 0.1% Formic acid in MeCN occurs between 0.00-3.00 minutes on the following columns: Acquity BEH C18 column (2.1×50 mm 1.7 Ipm) at 0.9 mL.

### Analytical LCMS

Method 1a: Waters X-Select CSH C18, 2.5 μm, 4.6×30 mm column eluting with a gradient of 0.1% Formic acid in MeCN in 0.1% Formic acid in water. The gradient from 5-95% 0.1% Formic acid in MeCN occurs between 0.00-3.00 minutes at 2.5 ml/min with a flush from 3.01-3.5 minutes at 4.5 ml/min. A column re-equilibration to 5% MeCN is from 3.60-4.00 minutes at 2.5 ml/min. UV spectra of the eluted peaks were measured using an Agilent 1260 Infinity or Agilent 1200 VWD at 254 nm. Mass spectra were measured using an Agilent 6120 or Agilent 1956 MSD running with positive/negative switching or an Agilent 6100 MSD running in either positive or negative mode.

### Example 1. Synthesis of 3-bromo-7-iodo-5,6,7,9-tetrahydro-8H-pyrido[2,3-b]azepin-8-one (compound 2)

### General Synthetic Scheme.

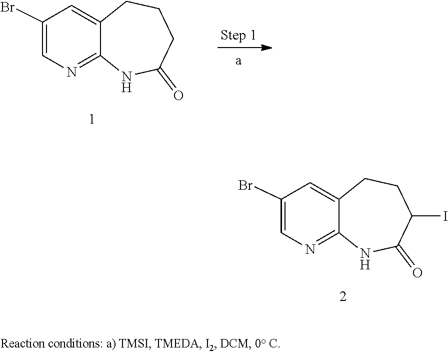 [SMILES: O=C1CCCc2cc(Br)cnc2N1 ; O=C1Nc2ncc(Br)cc2CCC1I]

3-Bromo-5,6,7,9-tetrahydro-8H-pyrido[2,3-b]azepin-8-one (compound 1) was prepared as described in AFFINIUM PHARMACEUTICALS, INC.—WO2007/67416, 2007, A2.

Step 1. 3-Bromo-7-iodo-5,6,7,9-tetrahydro-8H-pyrido[2,3-b]azepin-8-one (compound 2): To a stirred solution of 3-bromo-5,6,7,9-tetrahydro-8H-pyrido[2,3-b]azepin-8-one (710 mg, 2.95 mmol, 1.0 eq.) in DCM (5 mL) were added at 0° C., TMEDA (1.76 mL, 11.8 mmol, 4.0 eq.) and iodotrimethylsilane (0.907 mL, 6.18 mmol, 2.1 eq.). The reaction mixture was stirred at 0° C. for 15 min. Diiodine (2.25 g, 8.85 mmol, 3.0 eq.) was added at 0° C. and the reaction mixture was stirred at 0° C. for 4 h. The mixture was quenched with a sat. sol. of Na₂S₂O₃ (20 mL) and water (40 mL). The aqueous layer was extracted twice with DCM (2×50 mL). The combined organic layers were dried over Na₂SO₄ and concentrated to dryness. The orange solid was triturated with MeOH (10 mL) to afford the desired compound (763 mg, 2.08 mmol, 70.6%) as a white solid.

UPLC-MS: m/z=366.8/368.8 [M+H]⁺ (ES+); tᵣₑₜ=1.41 min (standard method)

### Example 2. Synthesis of N-Methyl-N-((2-methylbenzofuran-3-yl)methyl)acrylamide (compound 7)

### General Synthetic Scheme.

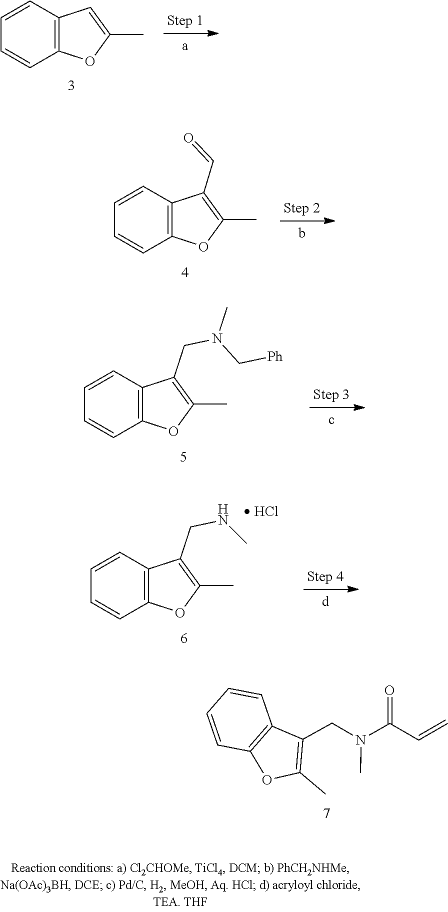

Step 1. 2-Methylbenzofuran-3-carbaldehyde (compound 4): To a solution of dichloro(methoxy)methane (5.10 mL, 56.7 mmol, 1.5 eq.) in DCM (100 mL) at 0° C. were added 2-methylbenzofuran (5.00 g, 37.8 mmol, 1.0 eq.) and then a 1M sol. in DCM of tin(IV) chloride (60.5 mL, 60.5 mmol, 1.6 eq.) over 30 mins.

After addition, the mixture was allowed to warm to rt over 30 mins, then poured into ice cold sat. sol. of NaHCO₃ (500 mL). The mixture was extracted twice with DCM (2×100 mL). The combined organic layers were dried over Na₂SO₄, concentrated in vacuo and purified by silica chromatography (0-50% EtOAc/isohexane) to afford the desired compound (5.30 g, 86%) as a yellow solid. ¹H NMR (400 MHz, CDCl₃): δ, ppm 10.16 (s, 1H), 8.06-8.01 (m, 1H), 7.41-7.35 (m, 1H), 7.34-7.23 (m, 2H), 2.70 (s, 3H).

Step 2. N-Benzyl-N-methyl-1-(2-methylbenzofuran-3-yl)methanamine (compound 5): To a solution of 2-methylbenzofuran-3-carbaldehyde (1.00 g, 6.24 mmol, 1.0 eq.) in DCE (20 mL) were added N-methyl-1-phenylmethanamine (0.98 mL, 7.49 mmol, 1.2 eq.) and sodium triacetoxyborohydride (1.99 g, 9.37 mmol, 1.5 eq.). The reaction mixture was stirred at rt for 72 hours, then washed with a sat. sol. of NaHCO₃ (20 mL), dried over over Na₂SO₄ and concentrated in vacuo to afford the desired compound (1.60 g, 94% yield) as a pale yellow oil which was used as such in the next step. ¹H NMR (400 MHz, DMSO-d₆): δ, ppm 7.63-7.58 (m, 1H), 7.48-7.43 (m, 1H), 7.33 (d, J=4.8 Hz, 4H), 7.28-7.20 (m, 3H), 3.56 (s, 2H), 3.52 (s, 2H), 2.42 (s, 3H), 2.08 (s, 3H).

Step 3. N-Methyl-1-(2-methylbenzofuran-3-yl)methanamine hydrochloride (compound 6): To a solution of N-benzyl-N-methyl-1-(2-methylbenzofuran-3-yl)methanamine (1.60 g, 6.03 mmol, 1.0 eq.) in MeOH (20 mL), were added a 1M aq. sol. of hydrochloric acid until pH=1 and Pd—C 87L 5% on carbon (0.64 g, 40% w/w). The reaction mixture was hydrogenated at 5 bars at rt for 18 hours, was filtered over celite and concentrated to dryness to afford the desired compound (737 mg, 56%) as a white solid. ¹H NMR (400 MHz, DMSO-d₆): δ, ppm 9.24 (s, 2H), 7.89-7.80 (m, 1H), 7.59-7.49 (m, 1H), 7.34-7.23 (m, 2H), 4.24 (s, 2H), 2.57 (s, 3H), 2.55 (s, 3H).

Step 4. N-Methyl-N-((2-methylbenzofuran-3-yl)methyl)acrylamide (compound 7): To a solution of N-methyl-1-(2-methylbenzofuran-3-yl)methanamine hydrochloride (300 mg, 1.42 mmol, 1.0 eq.) in THF (10 mL), were added triethylamine (600 μL, 4.25 mmol, 3.0 eq.) and dropwise at rt over 15 mins acryloyl chloride (154 mg, 1.70 mmol, 1.2 eq.). The reaction mixture was stirred at rt for 1 h, then poured into water (30 mL). The organic solvent was evaporated. The solid in suspension was collected by filtration, washed with water (10 mL) and dried to afford the desired compound (316 mg, 95%) as a colourless solid.

UPLC-MS: m/z=230.1 [M+H]⁺ (ES+); tᵣₑₜ=1.56 min (standard method)

### Example 3. (E)-N-methyl-3-(7-(methylamino)-8-oxo-6,7,8,9-tetrahydro-5H-pyrido[2,3-b]azepin-3-yl)-N-((2-methylbenzofuran-3-yl)methyl)acrylamide (compound 11)

### General Synthetic Scheme.

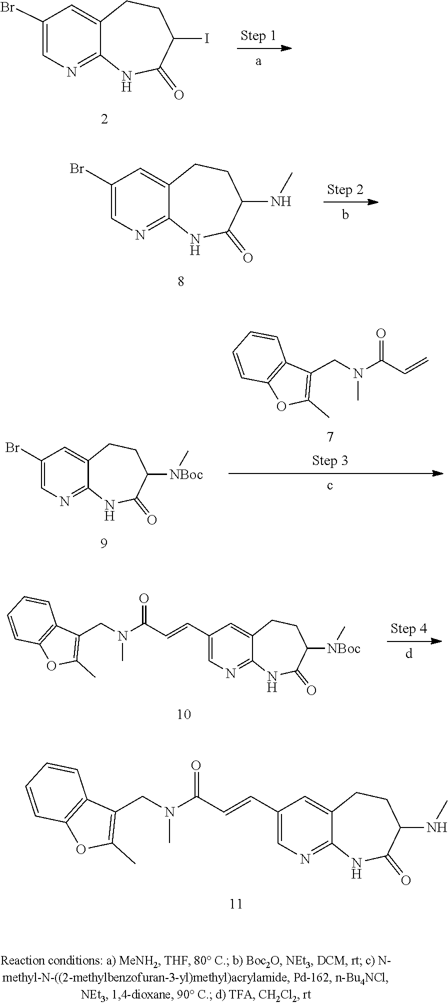 [SMILES: O=C1Nc2ncc(Br)cc2CCC1I ; CNC1CCc2cc(Br)cnc2NC1=O ; C=CC(=O)N(C)Cc1c(C)oc2ccccc12 ; CN(C(=O)OC(C)(C)C)C1CCc2cc(Br)cnc2NC1=O ; Cc1oc2ccccc2c1CN(C)C(=O)/C=C/c1cnc2c(c1)CCC(N(C)C(=O)OC(C)(C)C)C(=O)N2 ; CNC1CCc2cc(/C=C/C(=O)N(C)Cc3c(C)oc4ccccc34)cnc2NC1=O]

Step 1. 3-Bromo-7-(methylamino)-5,6,7,9-tetrahydropyrido[2,3-b]azepin-8-one (compound 8): To a stirred solution of 3-bromo-7-iodo-5,6,7,9-tetrahydro-8H-pyrido[2,3-b]azepin-8-one (320 mg, 0.872 mmol, 1.0 eq.) in THF (9.0 mL) was added a 2M solution of methylamine in THF (4.36 mL, 8.72 mmol, 10 eq.). The reaction was stirred at 80° C. for 1 h. The solvent was removed in vacuo, then the residue was diluted with a solution of K₂CO₃ (50 mL) and extracted twice with DCM (2×50 mL). The combined organic phases were dried over Na₂SO₄ and concentrated in vacuo to afford the desired compound (236 mg, 0.785 mmol, 90.0% yield) as a yellow solid.

UPLC-MS: m/z=270.0/272.0 [M+H]⁺ (ES+); tᵣₑₜ=0.93 min (standard method)

Step 2. tert-Butyl N-(3-bromo-8-oxo-5,6,7,9-tetrahydropyrido[2,3-b]azepin-7-yl)-N-methyl-carbamate (compound 9): To a stirred solution of 3-bromo-7-(methylamino)-5,6,7,9-tetrahydropyrido[2,3-b]azepin-8-on (230 mg, 0.851 mmol, 1.0 eq.) in DCM (8.5 mL) were added triethylamine (216 mg, 2.13 mmol, 2.5 eq.) and di-tert-butyl dicarbonate (197 mg, 0.894 mmol, 1.05 eq.). The reaction mixture was stirred at rt for 1 h then diluted with water (50 mL) and extracted twice with DCM (2×50 mL). The combined organic layers were dried over Na₂SO₄, concentrated in vacuo and purified by silica gel chromatography eluting with 0-70% EtOAc in n-heptane to afford the desired compound (202 mg, 0.546 mmol, 64.1% yield) as a white solid.

UPLC-MS: m/z=370.1/372.1 [M+H]⁺ (ES); tᵣₑₜ=1.66 min (standard method)

Step 3. tert-Butyl N-methyl-N-[3-[(E)-3-[methyl-[(2-methylbenzofuran-3-yl)methyl]amino]-3-oxo-prop-1-enyl]-8-oxo-5,6,7,9-tetrahydropyrido[2,3-b]azepin-7-yl]carbamate (compound 10): To a stirred solution of N-(3-bromo-8-oxo-5,6,7,9-tetrahydropyrido[2,3-b]azepin-7-yl)-N-methyl-carbamate (200 mg, 0.540 mmol, 1.0 eq.) in dioxane (1.5 mL) were added N-methyl-N-((2-methylbenzofuran-3-yl)methyl)acrylamide (186 mg, 0.810 mmol, 1.5 eq.), N-ethyl-N-isopropylpropan-2-amine (0.284 mL, 1.62 mmol, 3.0 eq.), tetrabutylammonium chloride (22.5 mg, 0.081 mmol, 0.15 eq.), and chloro(crotyl)(tri-tert-butylphosphine)palladium(II) (13.0 mg, 0.032 mmol, 0.06 eq.). The reaction mixture was flushed with N₂, stirred at 90° C. for 2 h, dioxane was evaporated and the residue was purified by silica gel chromatography eluting with 0-100% EtOAc in n-heptane to afford the desired compound (255 mg, 0.492 mmol, 91.0% yield) as a white solid.

UPLC-MS: m/z=519.3 [M+H]⁺ (ES+); tᵣₑₜ=1.94 min (standard method)

Step 4. (E)-N-Methyl-3-[7-(methylamino)-8-oxo-5,6,7,9-tetrahydropyrido[2,3-b]azepin-3-yl]-N-[(2-methylbenzofuran-3-yl)methyl]prop-2-enamide (compound 11): To a stirred solution of tert-butyl N-methyl-N-[3-[(E)-3-[methyl-[(2-methylbenzofuran-3-yl)methyl] amino]-3-oxo-prop-1-enyl]-8-oxo-5,6,7,9-tetrahydropyrido[2,3-b]azepin-7-yl]carbamate (262 mg, 0.505 mmol, 1.0 eq.) in DCM (1.5 mL) was added 2,2,2-trifluoroacetic acid (3.90 mL, 50.5 mmol, 100 eq.). The reaction mixture was stirred at rt for 1 h and was poured into Et₂O (100 mL). The white solid was filtered off, taken up with DCM (50 mL) and a sat. sol. of K₂CO₃ (50 mL). The aqueous layer was extracted once with DCM (50 mL). The combined organic layers were dried over Na₂SO₄, concentrated in vacuo and freeze dried to afford the desired compound (165 mg, 0.382 mmol, 75.7% yield) as a white solid.

¹H NMR (400 MHz, DMSO-d₆): δ 10.34 (s, 1H), 8.57-8.54 (rotamers, 1H), 8.21-8.16 (rotamers, 1H), 7.72-7.43 (m, 3H), 7.38-7.15 (m, 3H), 4.94-4.74 (rotamers, CH₂, 2H), 3.05-2.82 (rotamers, CH₃, 3H), 3.02-2.92 (m, 1H), 2.75-2.55 (m, 2H), 2.52-2.47 (rotamers, CH₃, 3H), 2.40-2.29 (m, 1H), 2.15 (s, 3H), 1.89-1.80 (m, 1H)

UPLC-MS: m/z=419.3 [M+H]⁺ (ES+); tᵣₑₜ=1.21 min (standard method)

### Example 4. Synthesis of (E)-3-[7-(dimethylamino)-8-oxo-5,6,7,9-tetrahydropyrido[2,3-b]azepin-3-yl]-N-methyl-N-[(2-methylbenzofuran-3-yl)methyl]prop-2-enamide (compound 13)

### General Synthetic Scheme.

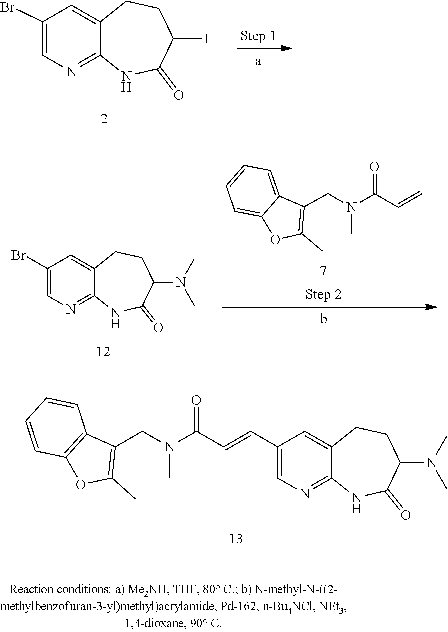 [SMILES: O=C1Nc2ncc(Br)cc2CCC1I ; C=CC(=O)N(C)Cc1c(C)oc2ccccc12 ; CN(C)C1CCc2cc(Br)cnc2NC1=O ; Cc1oc2ccccc2c1CN(C)C(=O)/C=C/c1cnc2c(c1)CCC(N(C)C)C(=O)N2]

Step 1. 3-Bromo-7-(dimethylamino)-5,6,7,9-tetrahydropyrido[2,3-b]azepin-8-one (compound 12): To a stirred solution of 3-bromo-7-iodo-5,6,7,9-tetrahydro-8H-pyrido[2,3-b]azepin-8-one (300 mg, 0.818 mmol, 1.0 eq.) in THF (8.0 mL) was added a 2M solution of dimethylamine in THF (4.09 mL, 8.17 mmol, 10 eq.). The reaction was stirred at 80° C. for 1 h, then the solvent was removed in vacuo, the residue was diluted with a solution of K₂CO₃ (50 mL) and extracted twice with DCM (2×50 mL). The combined organic phases were dried over Na₂SO₄, concentrated in vacuo and purified by silica gel chromatography eluting with 0-15% MeOH in DCM to afford the desired compound (165 mg, 0.523 mmol, 63.9% yield, 90% pure) as a white solid.

UPLC-MS: m/z=284.0/286.0 [M+H]⁺ (ES+); tᵣₑₜ=0.91 min (standard method)

Step 2. (E)-3-[7-(Dimethylamino)-8-oxo-5,6,7,9-tetrahydropyrido[2,3-b]azepin-3-yl]-N-methyl-N-[(2-methylbenzofuran-3-yl)methyl]prop-2-enamide (compound 13): To a stirred solution of 3-bromo-7-(dimethylamino)-5,6,7,9-tetrahydropyrido[2,3-b]azepin-8-one (165 mg, 0.581 mmol, 1.0 eq.) in dioxane (1.6 mL) were added N-methyl-N-((2-methylbenzofuran-3-yl)methyl)acrylamide (200 mg, 0.871 mmol, 1.5 eq.), N-ethyl-N-isopropylpropan-2-amine (0.305 mL, 1.74 mmol, 3.0 eq.), tetrabutylammonium chloride (24.2 mg, 0.087 mmol, 0.15 eq.), and chloro(crotyl)(tri-tert-butylphosphine)palladium(II) (13.9 mg, 0.035 mmol, 0.06 eq.). The reaction mixture was flushed with N₂, stirred at 90° C. for 2 h, dioxane was evaporated and the residue was purified by silica gel chromatography eluting with 0-10% MeOH in DCM to afford the desired compound pure at 90%. This was purified by silica gel chromatography eluting with 0-1% NEt₃ in DCM and was freeze dried to afford the desired compound pure at 95% (35.0 mg, 0.077 mmol, 13.2% yield) as a white solid.

¹H NMR (400 MHz, DMSO-d₆): δ 10.13 (s, 1H), 8.53-8.50 (rotamers, 1H), 8.18-8.13 (rotamers, 1H), 7.70-7.46 (m, 3H), 7.38-7.15 (m, 3H), 4.94-4.74 (rotamers, CH₂, 2H), 3.05-2.82 (rotamers, CH₃, 3H), 3.02-2.92 (m, 1H), 2.80-2.71 (m, 1H), 2.70-2.60 (m, 1H), 2.52-2.47 (rotamers, CH₃, 3H), 2.28 (s, 6H), 2.26-2.12 (m, 2H)

UPLC-MS: m/z=433.3 [M+H]⁺ (ES+); tᵣₑₜ=1.20 min (standard method)

### Example 5. Synthesis of (E)-N-methyl-N-[(2-methylbenzofuran-3-yl)methyl]-3-(8-oxo-7-pyrrolidin-1-yl-5,6,7,9-tetrahydropyrido[2,3-b]azepin-3-yl)prop-2-enamide (compound 15)

### General Synthetic Scheme.

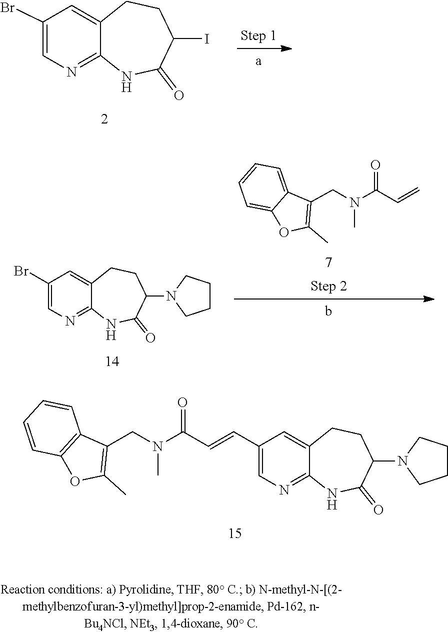 [SMILES: O=C1Nc2ncc(Br)cc2CCC1I ; C=CC(=O)N(C)Cc1c(C)oc2ccccc12 ; O=C1Nc2ncc(Br)cc2CCC1N1CCCC1 ; Cc1oc2ccccc2c1CN(C)C(=O)/C=C/c1cnc2c(c1)CCC(N1CCCC1)C(=O)N2]

Step 1. 3-Bromo-7-pyrrolidin-1-yl-5,6,7,9-tetrahydropyrido[2,3-b]azepin-8-one (compound 14): To a stirred solution of 3-bromo-7-iodo-5,6,7,9-tetrahydro-8H-pyrido[2,3-b]azepin-8-one (300 mg, 0.818 mmol, 1.0 eq.) in THF (8.0 mL) was added pyrolidine (0.689 mL, 8.17 mmol, 10 eq.). The reaction was stirred at 80° C. for 1 h, then, the solvent was removed in vacuo. The residue was diluted with a solution of K₂CO₃ (50 mL) and extracted twice with DCM (2×50 mL). The combined organic phases were dried over Na₂SO₄, concentrated in vacuo and purified by silica gel chromatography eluting with 0-15% MeOH in DCM to afford the desired compound (80.0 mg, 0.258 mmol, 31.5%) as a white solid.

UPLC-MS: m/z=310.0/312.0 [M+H]⁺ (ES+); tᵣₑₜ=0.58 min (standard method)

Step 2. (E)-N-methyl-N-[(2-methylbenzofuran-3-yl)methyl]-3-(8-oxo-7-pyrrolidin-1-yl-5,6,7,9-tetrahydropyrido[2,3-b]azepin-3-yl)prop-2-enamide (compound 15): To a stirred solution of 3-bromo-7-pyrrolidin-1-yl-5,6,7,9-tetrahydropyrido[2,3-b]azepin-8-one (80.0 mg, 0.258 mmol, 1.0 eq.) in dioxane (0.7 mL) were added N-methyl-N-((2-methylbenzofuran-3-yl)methyl)acrylamide (88.7 mg, 0.387 mmol, 1.5 eq.), N-ethyl-N-isopropylpropan-2-amine (0.135 mL, 0.774 mmol, 3.0 eq.), tetrabutylammonium chloride (10.8 mg, 0.039 mmol, 0.15 eq.), and chloro(crotyl)(tri-tert-butylphosphine)palladium(II) (6.20 mg, 0.0155 mmol, 0.06 eq.). The reaction mixture was flushed with N₂ and stirred at 90° C. for 3 h. Dioxane was evaporated and the residue was purified by silica gel chromatography eluting with 0-10% MeOH in DCM to afford the desired compound (72.0 mg, 0.157 mmol, 60.9% yield) as a white solid.

¹H NMR (400 MHz, DMSO-d₆): δ 10.13 (s, 1H), 8.57 (s, 1H), 8.24-8.19 (rotamers, 1H), 7.70-7.46 (m, 3H), 7.37-7.15 (m, 3H), 4.94-4.74 (rotamers, CH₂, 2H), 3.05-2.82 (rotamers, CH₃, 3H), 2.80-2.64 (m, 4H), 2.52-2.47 (rotamers, CH₃, 3H), 2.30-2.21 (m, 1H), 1.86-1-60 (m, 4H), 4 aliphatic protons not observed.

UPLC-MS: m/z=459.2 [M+H]+(ES+); tᵣₑₜ=1.23 min (standard method)

### Example 6. Synthesis of (E)-3-((2R,3S)-3-amino-2-methyl-4-oxo-2,3,4,5-tetrahydro-1H-pyrido[2,3-b][1,4]diazepin-8-yl)-N-methyl-N-((2-methylbenzofuran-3-yl)methyl)acrylamide (compound 23)

### General Synthetic Scheme.

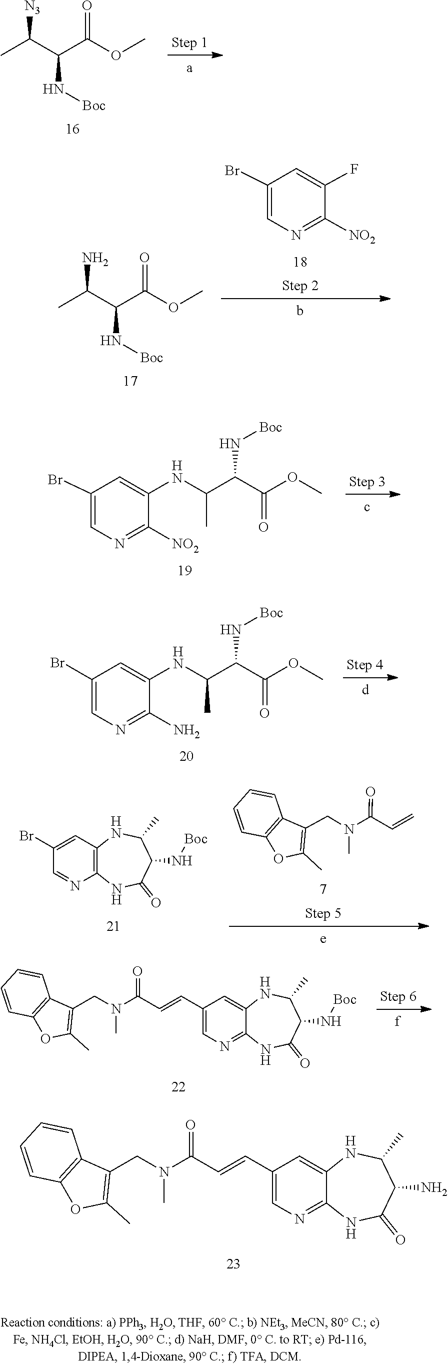

Methyl (2S,3R)-3-azido-2-((tert-butoxycarbonyl)amino)butanoate (compound 16) was prepared as described in J. Med. Chem. 2017, 60, 12, 5002-5014

Step 1. (2S,3R)-Methyl 3-amino-2-((tert-butoxycarbonyl)amino)butanoate (compound 17): Triphenylphosphine (0.65 g, 2.48 mmol, 2.0 eq.) and water (0.09 mL, 4.96 mmol, 4.0 eq.) were added to a stirred solution of (2S,3R)-methyl 3-azido-2-((tert-butoxycarbonyl)amino)butanoate (0.32 g, 1.24 mmol, 1.0 eq.) in THF (10 mL) and the reaction mixture was heated to 60° C. and stirred for 16 h. The reaction mixture was allowed to cool to rt, then NaHCO₃ (40 mL, sat. aq.) was added and the aqueous mixture was extracted with EtOAc (3×40 mL). The combined organic extracts were washed with brine (1×40 mL), dried using MgSO₄, concentrated in vacuo and applied to a SCX column. The SCX column was washed with MeOH (30 mL) and the product was eluted with methanolic ammonia and concentrated in vacuo to give the desired product as a colourless oil (0.24 g, 78%). ¹H NMR (500 MHz, DMSO-d₆) δ 6.97 (d, J=8.4 Hz, 1H), 3.88 (dd, J=8.4, 4.6 Hz, 1H), 3.62 (s, 3H), 3.16-3.10 (m, 1H), 1.52 (s, 2H), 1.39 (s, 9H), 0.97 (d, J=6.6 Hz, 3H).

Step 2. (2S,3R)-Methyl 3-((5-bromo-2-nitropyridin-3-yl)amino)-2-((tert-butoxycarbonyl)amino)butanoate (compound 19): A mixture of 5-bromo-3-fluoro-2-nitropyridine (0.22 g, 0.99 mmol, 1.0 eq.), (2S,3R)-methyl 3-amino-2-((tert-butoxycarbonyl)amino)butanoate (0.23 g, 0.99 mmol, 1.0 eq.) and triethylamine (0.55 mL, 3.96 mmol, 4.0 eq.) in MeCN (5 mL) was stirred at 80° C. for 5 h and at rt for 3 days. The reaction mixture was concentrated in vacuo and purified by column chromatography (0-50% EtOAc/isohexane) to give the desired product as a yellow oil (0.37 g, 82%).

R^{t} 1.80 min (Method la) m/z 377/379 (M-tBu)⁺ (ES⁺); ¹H NMR (500 MHz, DMSO-d₆) δ 7.98 (d, J=1.9 Hz, 1H), 7.88 (d, J=1.8 Hz, 1H), 7.73 (d, J=9.4 Hz, 1H), 7.68 (d, J=8.3 Hz, 1H), 4.44-4.35 (m, 1H), 4.33 (dd, J=8.4, 5.2 Hz, 1H), 3.59 (s, 3H), 1.37 (s, 9H), 1.23 (d, J=6.5 Hz, 3H).

Step 3. (2S,3R)-Methyl 3-((2-amino-5-bromopyridin-3-yl)amino)-2-((tert-butoxycarbonyl)amino)butanoate (compound 20): A mixture of (2S,3R)-methyl 3-((5-bromo-2-nitropyridin-3-yl)amino)-2-((tert-butoxycarbonyl)amino)butanoate (0.37 g, 0.85 mmol, 1.0 eq.), iron powder (0.38 g, 6.83 mmol, 8.0 eq.) and NH₄Cl (0.18 g, 3.42 mmol, 4.0 eq.) in a mixture of EtOH (10 mL) and H₂O (2.5 mL) was heated and stirred at 90° C. for 2 h. The reaction mixture was filtered through Celite©, the cake was washed with EtOH (50 mL) and the filtrate was concentrated in vacuo. The crude material was purified by column chromatography (0-100% EtOAc/isohexane) to give the desired product as a brown oil (0.22 g, 52%). R^{t} 1.43 min (Method la) m/z 403/405 (M+H)*(ES⁺); ¹H NMR (500 MHz, DMSO-d₆) δ 7.31 (d, J=2.0 Hz, 1H), 7.15 (d, J=9.3 Hz, 1H), 6.75 (d, J=2.1 Hz, 1H), 5.68 (s, 2H), 4.53 (d, J=9.7 Hz, 1H), 4.29 (dd, J=9.3, 3.8 Hz, 1H), 4.11-4.05 (m, 1H), 3.56 (s, 3H), 1.42 (s, 9H), 1.11 (d, J=6.5 Hz, 3H).

Step 4. tert-Butyl ((2R,3S)-8-bromo-2-methyl-4-oxo-2,3,4,5-tetrahydro-1H-pyrido[2,3-b][1,4]diazepin-3-yl)carbamate (compound 21): NaH 60% in mineral oil (50 mg, 1.23 mmol, 3.0 eq.) was added to a stirred solution of (2S,3R)-methyl 3-((2-amino-5-bromopyridin-3-yl)amino)-2-((tert-butoxycarbonyl)amino)butanoate (0.17 g, 0.41 mmol, 1.0 eq.) in DMF (5 mL) at 0° C. The reaction mixture was allowed to return to rt and was stirred for 1.5 h, then the reaction was quenched with water (50 mL). The resulting precipitate was collected by filtration to give the desired product as an off-white solid (72 mg, 47%). The aqueous filtrate was then extracted with EtOAc (3×100 mL) and the combined organic layers were washed with brine (1×50 mL), dried with MgSO₄ and concentrated in vacuo. The crude material was purified by column chromatography (EtOAc/isohexane) to give a further portion of the desired product as a white solid (36 mg, 23%). R^{t} 1.91 min (Method la) m/z 315/317 (M-tBu)⁺ (ES⁺); ¹H NMR (500 MHz, DMSO-d₆) δ 10.29 (s, 1H), 7.77 (d, J=2.1 Hz, 1H), 7.32 (d, J=2.1 Hz, 1H), 6.82 (d, J=7.6 Hz, 1H), 6.47 (d, J=6.4 Hz, 1H), 4.38-4.26 (m, 1H), 3.83-3.69 (m, 1H), 1.38 (s, 9H), 1.10 (d, J=6.5 Hz, 3H).

Step 5. tert-butyl ((2R,3S)-2-methyl-8-((E)-3-(methyl((2-methylbenzofuran-3-yl)methyl)amino)-3-oxoprop-1-en-1-yl)-4-oxo-2,3,4,5-tetrahydro-1H-pyrido[2,3-b][1,4]diazepin-3-yl)carbamate (compound 22): To a stirred solution of tert-butyl ((2R,3S)-8-bromo-2-methyl-4-oxo-2,3,4,5-tetrahydro-1H-pyrido[2,3-b][1,4]diazepin-3-yl)carbamate (105 mg, 0.282 mmol, 1.0 eq.) in dioxane (2.5 mL) were added N-methyl-N-((2-methylbenzofuran-3-yl)methyl)acrylamide (97.0 mg, 0.424 mmol, 1.5 eq.), N-ethyl-N-isopropylpropan-2-amine (0.133 mL, 0.764 mmol, 2.7 eq.) and chloro(crotyl)(tri-tert-butylphosphine)palladium(II) (11.3 mg, 0.028 mmol, 0.10 eq.). The reaction mixture was flushed with N₂, stirred at 90° C. for 2 h, dioxane was evaporated and the residue was purified by silica gel chromatography eluting with 0-3% MeOH in DCM to afford the desired compound (127 mg, 0.244 mmol, 86.0% yield) as a yellow solid.

UPLC-MS: m/z=464.1 [M+H]⁺ (ES+); tᵣₑₜ=2.50 min (method 1b)

Step 6. (E)-3-((2R,3S)-3-amino-2-methyl-4-oxo-2,3,4,5-tetrahydro-1H-pyrido[2,3-b][1,4]diazepin-8-yl)-N-methyl-N-((2-methylbenzofuran-3-yl)methyl)acrylamide (compound 23): tert-Butyl ((2R,3S)-2-methyl-8-((E)-3-(methyl ((2-methyl benzofuran-3-yl)methyl)amino)-3-oxoprop-1-en-1-yl)-4-oxo-2,3,4,5-tetra hydro-1H-pyrido[2,3-b][1,4]diazepin-3-yl)carbamate (100 mg, 0.192 mmol, 1.0 eq.) was dissolved in dichloromethane (3 mL) and trifluoroacetic acid (2 ml, 26 mmol, 135 eq.). The reaction mixture was stirred at rt for 10 min. The reaction mixture was evaporated under reduced pressure, the residue dissolved in DCM (30 mL) and washed with a sat. sol. of NaHCO₃ (30 mL).

The DCM phase was dried through a phase separation column and evaporated under reduced pressure. The crude product was purified by reverse phase purification (Biotage Isolera, 12 g RP column C18 cartridge; gradient 5-80% (acetonitrile+0.1% formic acid) in (water+0.1% formic acid) over 14 CV) and the clean fractions lyophilised to afford the desired product (59 mg, 0.140 mmol, 73% yield).

UPLC-MS: m/z=420.2 [M+H]⁺ (ES+); tᵣₑₜ=1.34 min (method 1b)

¹H NMR (400 MHz, DMSO-d₆): δ 1.10 (d, 3H), 2.52-2.48 (underneath DMSO residual solvent peak, 3H), 3.00 (s, 3H), 3.64-3.62 (m, 1H), 3.70-3.66 (m, 1H), 4.77 (s, 2H), 5.81 (d, 1H), 7.27-7.03 (m, 3H), 7.41 (d, 1H), 7.50-7.43 (m, 2H), 7.54 (d, 1H), 8.00 (d, 1H), 8.14 (s, 1H), 9.62 (s, 1H).

### Example 7. Synthesis of (S*,E)-N-methyl-N-((2-methylbenzofuran-3-yl)methyl)-3-(8-oxo-7-(pyrrolidin-1-yl)-6,7,8,9-tetrahydro-5H-pyrido[2,3-b]azepin-3-yl)acrylamide (compound 25; * stereochemistry arbitrarily assigned)

### General Synthetic Scheme.

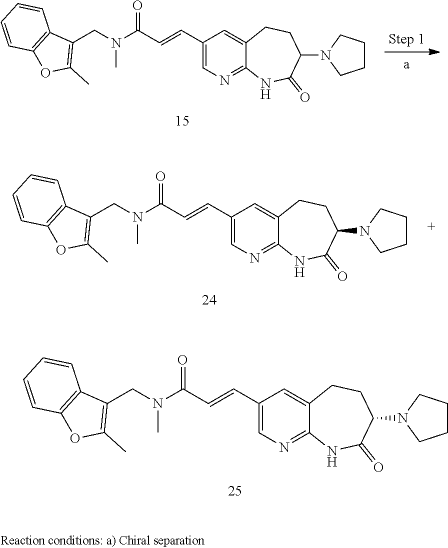 [SMILES: Cc1oc2ccccc2c1CN(C)C(=O)/C=C/c1cnc2c(c1)CCC(N1CCCC1)C(=O)N2 ; Cc1oc2ccccc2c1CN(C)C(=O)/C=C/c1cnc2c(c1)CC[C@@H](N1CCCC1)C(=O)N2 ; Cc1oc2ccccc2c1CN(C)C(=O)/C=C/c1cnc2c(c1)CC[C@H](N1CCCC1)C(=O)N2]

Step 1. Chiral separation—(S*,E)-N-methyl-N-((2-methylbenzofuran-3-yl)methyl)-3-(8-oxo-7-(pyrrolidin-1-yl)-6,7,8,9-tetrahydro-5H-pyrido[2,3-b]azepin-3-yl)acrylamide (compound 25; * stereochemistry arbitrarily assigned): The racemate (E)-N-methyl-N-((2-methylbenzofuran-3-yl)methyl)-3-(8-oxo-7-(pyrrolidin-1-yl)-6,7,8,9-tetrahydro-5H-pyrido[2,3-b]azepin-3-yl)acrylamide (54.6 mg, 0.119 mmol) was purified on SFS using Chiralpak IH (20 mm×250 mm, 5 μm) with a flowrate of 50 mL/min and the eluent (45/55 EtOH/CO₂ with 0.2% of NH₃) to afford the first eluting enantiomer arbitrary assigned as compound 25 (16.1 mg, 0.035 mmol, 29.4% yield, ee: 99.6%) as a beige solid.

UPLC-MS: m/z=459.4 [M+H]⁺ (ES+); tᵣₑₜ=1.22 min (standard method)

¹H NMR (400 MHz, DMSO-d₆): δ 10.13 (s, 1H), 8.57 (s, 1H), 8.24-8.19 (rotamers, 1H), 7.70-7.46 (m, 3H), 7.37-7.15 (m, 3H), 4.94-4.74 (rotamers, CH₂, 2H), 3.05-2.82 (rotamers, CH₃, 3H), 2.80-2.64 (m, 4H), 2.52-2.47 (rotamers, CH₃, 3H), 2.30-2.21 (m, 1H), 1.86-1-60 (m, 4H), 4 aliphatic protons not observed.

### Example 8. Synthesis of (E)-3-((R*)-7-((R)-3-hydroxypyrrolidin-1-yl)-8-oxo-6,7,8,9-tetrahydro-5H-pyrido[2,3-b]azepin-3-yl)-N-methyl-N-((2-methylbenzofuran-3-yl)methyl)acrylamide (compound 28; * stereochemistry arbitrarily assigned)

General Synthetic Scheme.

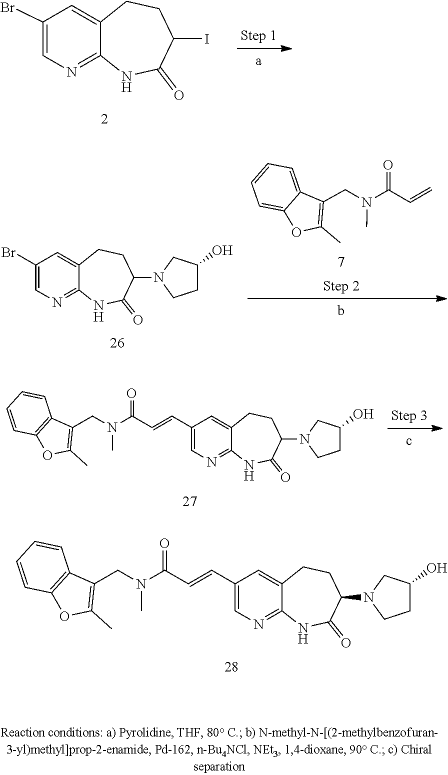 [SMILES: O=C1Nc2ncc(Br)cc2CCC1I ; C=CC(=O)N(C)Cc1c(C)oc2ccccc12 ; O=C1Nc2ncc(Br)cc2CCC1N1CC[C@@H](O)C1 ; Cc1oc2ccccc2c1CN(C)C(=O)/C=C/c1cnc2c(c1)CCC(N1CC[C@@H](O)C1)C(=O)N2 ; Cc1oc2ccccc2c1CN(C)C(=O)/C=C/c1cnc2c(c1)CC[C@@H](N1CC[C@@H](O)C1)C(=O)N2]

Step 1. 3-Bromo-7-((R)-3-hydroxypyrrolidin-1-yl)-5,6,7,9-tetrahydro-8H-pyrido[2,3-b]azepin-8-one (compound 26): To a stirred solution of 3-bromo-7-iodo-5,6,7,9-tetrahydro-8H-pyrido[2,3-b]azepin-8-one (150 mg, 0.409 mmol, 1.0 eq.) in THF (4.0 mL) was added (R)-pyrrolidin-3-ol (363 mg, 4.09 mmol, 10 eq.). The reaction was stirred at 80° C. for 1 h, then, the solvent was removed in vacuo. The residue was diluted with a solution of K₂CO₃ (50 mL) and extracted twice with DCM (2×50 mL). The combined organic phases were dried over Na₂SO₄, concentrated in vacuo, and purified by silica gel chromatography eluting with 0-30% MeOH in DCM to afford the desired compound (155.0 mg, 0.356 mmol, 87.2%) as a white solid.

UPLC-MS: m/z=326.0/328.0 [M+H]⁺ (ES+); tᵣₑₜ=0.54 min (standard method)

Step 2. (E)-3-(7-((R)-3-hydroxypyrrolidin-1-yl)-8-oxo-6,7,8,9-tetrahydro-5H-pyrido[2,3-b]azepin-3-yl)-N-methyl-N-((2-methylbenzofuran-3-yl)methyl)acrylamide (compound 27): To a stirred solution of 3-bromo-7-((R)-3-hydroxypyrrolidin-1-yl)-5,6,7,9-tetrahydro-8H-pyrido[2,3-b]azepin-8-one (155 mg, 0.475 mmol, 1.0 eq.) in dioxane (1.3 mL) were added N-methyl-N-((2-methylbenzofuran-3-yl)methyl)acrylamide (163 mg, 0.713 mmol, 1.5 eq.), N-ethyl-N-isopropylpropan-2-amine (0.250 mL, 1.426 mmol, 3.0 eq.), tetrabutylammonium chloride (19.8 mg, 0.071 mmol, 0.15 eq.), and chloro(crotyl)(tri-tert-butylphosphine)palladium(II) (11.4 mg, 0.029 mmol, 0.06 eq.). The reaction mixture was flushed with N₂ and stirred at 90° C. for 3 h. Dioxane was evaporated, and the residue was purified by silica gel chromatography eluting with 0-10% MeOH in DCM to afford the desired compound (226 mg, 0.475 mmol, quantitative yield) as a white solid.

UPLC-MS: m/z=476.2 [M+H]⁺ (ES+); tᵣₑₜ=1.09 min (standard method)

Step 3. Chiral separation—(E)-3-((R*)-7-((R)-3-hydroxypyrrolidin-1-yl)-8-oxo-6,7,8,9-tetrahydro-5H-pyrido[2,3-b]azepin-3-yl)-N-methyl-N-((2-methylbenzofuran-3-yl)methyl)acrylamide (compound 28; * stereochemistry arbitrarily assigned): The mixture of diastereoisomers (E)-3-(7-((R)-3-hydroxypyrrolidin-1-yl)-8-oxo-6,7,8,9-tetrahydro-5H-pyrido[2,3-b]azepin-3-yl)-N-methyl-N-((2-methylbenzofuran-3-yl)methyl)acrylamide (226 mg, 0.475 mmol) was purified on liquid chromatography using Daicel Chiralpak IBN-5 (250*10 mm) with a flowrate of 5 mL/min and an isocratic elution (1/1 MeOH/EtOH with 0.1% of diethylamine) to afford the first eluting diastereoisomer arbitrarily assigned as compound 28 (35.0 mg, 0.074 mmol, 15.5% yield, de: 93.64%) as a white solid.

UPLC-MS: m/z=476.2 [M+H]⁺ (ES+); tᵣₑₜ=1.09 min (standard method)

¹H NMR (400 MHz, 80° C., DMSO-d₆): δ 9.76 (broad s, 1H), 8.46 (d, J=4.0 Hz, 1H), 8.01 (d, J=4.0 Hz, 1H), 7.58-7.51 (m, 2H), 7.46-7.44 (m, 1H), 7.25-7.16 (m, 3H), 4.79 (s, 2H), 4.32 (broad s, 1H), 4.11 (broad s, 1H), 3.25-3.00 (m, 3H), 2.93-2.85 (m, 1H), 2.80-2.60 (m, 5H), 2.37-2.17 (m, 2H), 1.90-1.79 (m, 1H), 1.54-1.45 (m, 1H). 4 aliphatic protons not visible.

### Example 9. Synthesis of (S*,E)-3-(7-amino-7-methyl-8-oxo-6,7,8,9-tetrahydro-5H-pyrido[2,3-b]azepin-3-yl)-N-methyl-N-((2-methylbenzofuran-3-yl)methyl)acrylamide (compound 39; * stereochemistry arbitrarily assigned)

### General Synthetic Scheme.

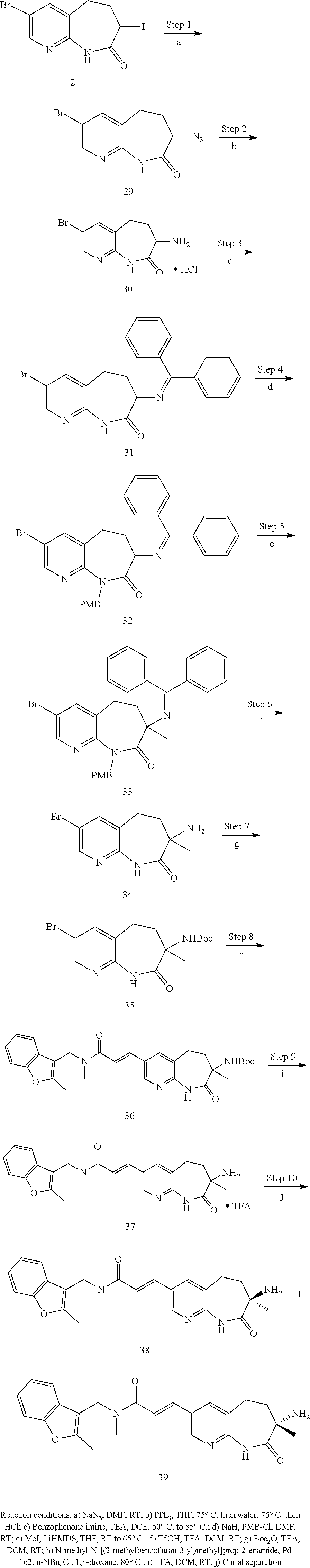

Step 1. (E)-3-((2R,3S)-3-amino-2-methyl-4-oxo-2,3,4,5-tetrahydro-1H-pyrido[2,3-b][1,4]diazepin-8-yl)-N-methyl-N-((2-methylbenzofuran-3-yl)methyl)acrylamide (compound 29): To an argon-purged solution of 7-iodo-5H,6H,7H,8H,9H-pyrido[2,3-b]azepin-8-one (1 eq., 2.71 g, 9.407 mmol) in DMF (45.5 mL) was added sodium azide (8 eq., 4.89 g, 75.25 mmol) and the reaction mixture was stirred at room temperature for 30 min. The resulting dark solution was diluted with water (40 mL) and EtOAc (60 mL) was added. The two layers were separated, and the aqueous layer was extracted with EtOAc (60 mL). The combined organic layers were washed with brine (3×50 mL), dried over Na₂SO₄, filtered, and concentrated under reduced pressure to afford the desired compound (1.78 g, 8.76 mmol, 93%) as an orange solid.

LC-MS: (ESI)⁺ m/z [M+H]⁺=204.06 (retention time 1.48 min)

¹H NMR (400 MHz, DMSO-d₆): 10.5 (broad s, 1H), 8.28 (dd, 1H, J=4.7 Hz, J=1.8 Hz), 7.72 (dd, 1H, J=7.5 Hz, J=1.8 Hz), 7.15 (dd, 1H, J=7.5 Hz, J=4.9 Hz), 4.03 (dd, 1H, J=11.3 Hz, J=8.0 Hz), 2.77-2.66 (m, 2H), 2.46-2.40 (m, 1H), 2.16-2.08 (m, 1H).

Step 2. 7-Amino-5H,6H,7H,8H,9H-pyrido[2,3-b]azepin-8-one hydrochloride (compound 30): A solution of 7-azido-5H,6H,7H,8H,9H-pyrido[2,3-b]azepin-8-one (1 eq., 0.86 g, 4.21 mmol) and triphenylphosphine (1.1 eq, 1.21 g, 4.63 mmol) in THF (32.49 mL) was heated at 75° C. for 1 h. Then water (8.55 mL) was added, and the reaction mixture was heated at 75° C. for 2 h. The solvent was removed under reduced pressure and aqueous solution of HCl (1M, 8 mL) and EtOAc (15 mL) were added. The two layers were separated, and the aqueous layer was extracted with EtOAc (2×15 mL). The aqueous layer was concentrated under reduced pressure and co-evaporated twice with EtOH to afford the desired compound (860 mg, 4.025 mmol, 96%) as a beige solid.

LC-MS: (ESI)⁺ m/z [M-HCl+H]⁺=178.09 (retention time 0.53 min)

¹H NMR (400 MHz, DMSO-d₆): 10.8 (broad s, 1H), 8.40 (broad s, 3H), 8.33 (dd, 1H, J=5.0 Hz, J=1.8 Hz), 7.81 (dd, 1H, J=7.6 Hz, J=1.8 Hz), 7.22 (dd, 1H, J=7.5 Hz, J=4.9 Hz), 3.80-3.73 (m, 1H), 2.84-2.79 (m, 1H), 2.76-2.70 (m, 1H), 2.62-2.56 (m, 1H), 2.22-2.14 (m, 1H).

Step 3. 3-Bromo-7-((diphenylmethylene)amino)-5,6,7,9-tetrahydro-8H-pyrido[2,3-b]azepin-8-one (compound 31): To a suspension of 7-amino-3-bromo-5,6,7,9-tetrahydro-8H-pyrido[2,3-b]azepin-8-one, HCl (1.53 g, 5.00 mmol) in 1,2-DCE (20 mL) at RT was added TEA (0.76 mL, 5.50 mmol) and the reaction was stirred at 50° C. for 15 min. Then, a solution of diphenylmethanimine (1.35 g, 7.46 mmol) in 1,2-DCE (10 mL) was added to the reaction mixture and the reaction was stirred at 85° C. for 18 h. The reaction was cooled to RT. The solvent was evaporated under reduced pressure and the crude was triturated twice with TBME/iso-hexane (1:1, 20 mL). The solids were filtered and dried under reduced pressure to afford the title compound as a tan solid (1.57 g, 71%).

Rt 1.93 min (Method 1b)

m/z 421 (M+H)*(ES⁺)

¹H NMR (500 MHz, DMSO-d₆) δ 10.18 (s, 1H), 8.31 (d, J=2.4 Hz, 1H), 7.90 (d, J=2.4 Hz, 1H), 7.56-7.35 (m, 4H), 7.30 (t, J=7.8 Hz, 2H), 7.22-7.17 (m, 2H), 7.09-7.03 (m, 2H), 3.97 (t, J=6.4 Hz, 1H), 2.86-2.71 (m, 2H), 2.36 (h, J=7.0 Hz, 1H), 2.23 (h, J=7.1 Hz, 1H).

Step 4. 3-Bromo-7-((diphenylmethylene)amino)-9-(4-methoxybenzyl)-5,6,7,9-tetrahydro-8H-pyrido[2,3-b]azepin-8-one (compound 32): To a stirred solution of 3-bromo-7-((diphenylmethylene)amino)-5,6,7,9-tetrahydro-8H-pyrido[2,3-b]azepin-8-one (0.7 g, 1.6 mmol) in dry DMF (14 mL) under a N₂ atmosphere at RT was added NaH (60% dispersion in mineral oil, 82 mg, 2.1 mmol). The reaction mixture was stirred at RT for 30 min. 1-(Chloromethyl)-4-methoxybenzene (0.29 mL, 2.06 mmol) was added and the reaction was stirred at RT for 18 h. Then, the reaction mixture was added to H₂O (150 mL) and the product was extracted using EtOAc (3×50 mL). The organic layers were combined and washed with H₂O (100 mL) and brine (100 mL). The organic phase was dried over anhydrous Na₂SO₄, and the solvent was removed under reduced pressure to afford the title compound as a brown solid (0.86 g, 88%).

Rt 2.43 min (Method 1b)

m/z 541/543 (M+H)*(ES⁺)

¹H NMR (500 MHz, DMSO-d₆) δ 8.46 (d, J=2.4 Hz, 1H), 7.91 (d, J=2.4 Hz, 1H), 7.45-7.38 (m, 4H), 7.30 (t, J=7.7 Hz, 2H), 7.20-7.16 (m, 2H), 7.16-7.11 (m, 2H), 7.02-6.93 (m, 2H), 6.81-6.76 (m, 2H), 5.09-4.91 (m, 2H), 4.08 (s, 1H), 3.68 (s, 3H), 2.70-2.52 (m, 2H), 2.43-2.19 (m, 2H).

Step 5. 3-Bromo-7-((diphenylmethylene)amino)-9-(4-methoxybenzyl)-7-methyl-5,6,7,9-tetrahydro-8H-pyrido[2,3-b]azepin-8-one (compound 33): To a microwave reaction vial, LiHMDS (1 M in THF, 1.79 mL, 1.79 mmol) was added to a solution of 3-bromo-7-((diphenylmethylene)amino)-9-(4-methoxybenzyl)-5,6,7,9-tetrahydro-8H-pyrido[2,3-b]azepin-8-one (0.73 g, 1.19 mmol) in THF (15 mL). The reaction was stirred for 15 min. Iodomethane (0.22 mL, 3.56 mmol) was added, the reaction vial was sealed, and the reaction was stirred for 3 h at 65° C. The reaction was allowed to cool to RT. The reaction mixture was then added to H₂O (30 mL), and the product was extracted using EtOAc (3×30 mL). The organic layers were combined, washed with brine (50 mL), and dried over anhydrous Na₂SO₄. The solvent was evaporated under reduced pressure to afford the title compound as a brown gum (0.77 g, 93%).

Rt 2.60 min (Method 1b)

m/z 553/555 (M+H)*(ES⁺)

¹H NMR (500 MHz, DMSO-d₆) δ 8.15 (d, J=2.4 Hz, 1H), 8.07 (d, J=2.4 Hz, 1H), 7.54-7.41 (m, 3H), 7.37-7.26 (m, 3H), 7.20-7.12 (m, 2H), 7.05-6.98 (m, 2H), 6.88-6.79 (m, 2H), 6.79-6.71 (m, 2H), 4.93-4.73 (m, 2H), 3.65 (s, 3H), 2.78 (td, J=13.1, 7.4 Hz, 1H), 2.65 (dd, J=13.4, 7.0 Hz, 1H), 2.48-2.38 (m, 1H), 2.32 (dd, J=13.1, 8.1 Hz, 1H), 1.27 (s, 3H).

Step 6. 7-Amino-3-bromo-7-methyl-5,6,7,9-tetrahydro-8H-pyrido[2,3-b]azepin-8-one (compound 34): To a solution of 3-bromo-7-((diphenylmethylene)amino)-9-(4-methoxybenzyl)-7-methyl-5,6,7,9-tetrahydro-8H-pyrido[2,3-b]azepin-8-one (0.57 g, 0.82 mmol) in DCM (10 mL) was added TfOH (0.73 mL, 8.15 mmol) and TFA (1.30 mL, 16.9 mmol) and the reaction was stirred for 4 h at RT. Then, H₂O (8 mL) was added, and the reaction was stirred vigorously for 2 h at RT. The aqueous layer was extracted with DCM (2×15 mL) and the organic extracts were discarded. The aqueous layer was then basified with sat. aq. NaHCO₃ solution (40 mL) and the product was extracted with DCM (3×30 mL). The combined organic layers were washed with brine (50 mL), dried over anhydrous Na₂SO₄, filtered and the solvent was removed under reduced pressure to afford the title compound as an off-white solid (0.17 g, 71%).

Rt 0.77 min (Method 2b)

m/z 270/272 (M+H)*(ES⁺)

¹H NMR (500 MHz, DMSO-d₆) δ 9.94 (s, 1H), 8.26 (d, J=2.4 Hz, 1H), 7.87 (d, J=2.4 Hz, 1H), 2.70 (t, J=6.9 Hz, 2H), 2.06-1.94 (m, 2H), 1.73 (s, 2H), 1.03 (s, 3H).

Step 7. tert-Butyl (3-bromo-7-methyl-8-oxo-6,7,8,9-tetrahydro-5H-pyrido[2,3-b]azepin-7-yl)carbamate (compound 35): To a solution of 7-amino-3-bromo-7-methyl-5,6,7,9-tetrahydro-8H-pyrido[2,3-b]azepin-8-one (0.17 g, 0.58 mmol) in DCM (4 mL) was added TEA (0.40 mL, 2.90 mmol). The mixture was stirred at RT for 15 min. Then, a solution of Boc₂O (0.19 g, 0.87 mmol) in DCM (1 mL) was added and the reaction was stirred at RT for 3 h. The reaction mixture was concentrated under reduced pressure at 45° C. The crude product was purified by chromatography (0-100% EtOAc/iso-hexane (+2% TEA)) to afford the title compound as a pale-yellow solid (0.11 g, 36%).

Rt 1.17 min (Method 2b)

m/z 270/272 (M+H-Boc)⁺ (ES⁺)

¹H NMR (500 MHz, DMSO-d₆) δ 9.81 (s, 1H), 8.21 (d, J=2.4 Hz, 1H), 7.77 (d, J=2.4 Hz, 1H), 6.61 (s, 1H), 2.77-2.66 (m, 1H), 2.63-2.56 (m, 1H), 2.44-2.32 (m, 1H), 2.06-1.99 (m, 1H), 1.24 (s, 9H), 1.21 (s, 3H).

Step 8. tert-Butyl (E)-(7-methyl-3-(3-(methyl((2-methylbenzofuran-3-yl)methyl)amino)-3-oxoprop-1-en-1-yl)-8-oxo-6,7,8,9-tetrahydro-5H-pyrido[2,3-b]azepin-7-yl)carbamate (compound 36): In a microwave vial under nitrogen, were added chloro(crotyl)(tri-tert-butylphosphine)palladium(II) (2.98 mg, 0.0074 mmol, 0.05 eq.), tetrabutylammonium chloride (1.24 mg, 0.0045 mmol, 0.03 eq.), N-methyl-N-[(2-methylbenzofuran-3-yl)methyl]prop-2-enamide (37.46 mg, 0.16 mmol, 1.1 eq.) and tert-butyl N-(3-bromo-7-methyl-8-oxo-6,9-dihydro-5H-pyrido[2,3-b]azepin-7-yl)carbamate (55. mg, 0.15 mmol, 1 eq.).

Then, 1,4-dioxane (0.7428 mL) and dry N-ethyl-N-isopropylpropan-2-amine (0.08 mL, 0.45 mmol, 3 eq.) were added and the mixture was stirred at 80° C. for 1 hour. Chloro(crotyl)(tri-tert-butylphosphine)palladium(II) (8.94 mg, 0.02 mmol, 0.15 eq.) was added and the mixture was stirred at 80° C. for 90 min. The solution was evaporated and the residue was quickly purified on a pad of silica gel eluting with 10-100% EtOAc in n-heptane. The purified fraction containing the desired compound (77 mg, 0.149 mmol, 77%) was directly used for the next step.

UPLC-MS: m/z=519.2 [M+H]⁺ (ES+); tᵣₑₜ=1.82 min (standard method)

Step 9. (E)-3-(7-amino-7-methyl-8-oxo-6,7,8,9-tetrahydro-5H-pyrido[2,3-b]azepin-3-yl)-N-methyl-N-((2-methylbenzofuran-3-yl)methyl)acrylamide trifluoroacetic acid (compound 37): In a round-bottom flask containing tert-butyl N-[7-methyl-3-[(E)-3-[methyl-[(2-methylbenzofuran-3-yl)methyl]amino]-3-oxo-prop-1-enyl]-8-oxo-6,9-dihydro-5H-pyrido[2,3-b]azepin-7-yl]carbamate (77.04 mg, 0.15 mmol, 1 eq.) were added DCM (4 mL) and trifluoroacetic acid (4 mL). The mixture was stirred at RT for 15 min then evaporated and the residue was purified on silica gel eluting with 5-100% MeCN (+0.1% TFA) in water (0.1% TFA) to afford the desired product (48 mg, 0.088 mmol, 79%) as a white powder after freeze-drying.

UPLC-MS: m/z=419.2 [M+H]⁺ (ES+); tᵣₑₜ=1.20 min (standard method)

Step 10. (S*,E)-3-(7-amino-7-methyl-8-oxo-6,7,8,9-tetrahydro-5H-pyrido[2,3-b]azepin-3-yl)-N-methyl-N-((2-methylbenzofuran-3-yl)methyl)acrylamide trifluoroacetic acid (compound 39; * stereochemistry arbitrarily assigned): (E)-3-(7-amino-7-methyl-8-oxo-6,7,8,9-tetrahydro-5H-pyrido[2,3-b]azepin-3-yl)-N-methyl-N-((2-methylbenzofuran-3-yl)methyl)acrylamide trifluoroacetic acid was purified on Chiral PAK-IC eluting with DCM/MeOH (50/50) to afford the desired product after freeze-drying (3.61 mg) as a white solid. Chirality was arbitrarily assigned.

UPLC-MS: m/z=419.2 [M+H]⁺ (ES+); tᵣₑₜ=1.20 min (standard method)

¹H NMR (500 MHz, DMSO-d₆) δ 10.92 (s, 1H), 8.64-8.60 (m, 1H), 8.30, (braod s, 3H), 8.23-8.18 (m, 1H), 7.75-7.45 (m, 3H), 7.40-7.13 (m, 3H), 4.94-4.75 (rotamers, CH₂), 3.06-2.88 (rotamers, CH₃), 2.86-2.80 (m, 2H), 2.55-2.52 (rotamers, CH₃), 2.44-2.40 (m, 1H), 2.28-2.20 (m, 1H), 1.13-1.11 (rotamers, CH₃).

### Example 10. Antibacterial Activity

The exemplified compounds were tested for activity on the target enzyme and on the bacteria, relying on the following test procedures:

### Inhibition of Fabl Proteins:

Inhibition of Fabl enzyme from Acinetobacter baumannii and Escherichia coli was tested by measuring the rate of NADH consumption (Aabsorbance at 340 nm/min) at 30° C. in 96-well plate format using an automated plate reader in the presence or absence of the test compounds. The assay mixture contained 100 mM Tris-HCl, pH 7.25 (A. baumannii) or 7.5 (E. coli), 100 mM ammonium acetate, 0.02% (A. baumannii) or 0.05% (E. coli) Pluronic F-68, 25 μM crotonyl ACP, 50 μM NADH, 25 μM (A. baumannii) or 50 μM (E. coli) recombinant Fabl protein, and 7.5% DMSO. Test compounds were added at concentrations ranging from 0.17 to 10,000 nM in a final well volume of 100 μl. This dose-response inhibitory assay was performed using a 10-point, serial dilution series for each test compound. IC50 values for each test compound were assigned from logistical sigmoid curve-fitting of the inhibition dose response curves.

### MIC:

The antibacterial activity of Fabl inhibitors against select Gram-negative and Gram-positive bacterial species including susceptible and multi-drug resistant A. baumannii, E. coli, K. pneumoniae and S. aureus was tested using the broth microdilution Minimal Inhibitory Concentration (MIC) assay following CLSI guidelines for insoluble compounds. Test articles were serially diluted 2-fold in 100% dimethyl sulfoxide (DMSO) and then diluted 100-fold into cation adjusted Mueller-Hinton broth (CA-MHB) to achieve a 10-point test concentration range in 1% DMSO. Final compound concentrations were 0.016-8 μg/ml for S. aureus or 0.06-32 μg/ml for the Gram-negative species. MIC test plates were then prepared by transferring 100 μl of the final assay medium (test article in CA-MHB, 1% DMSO) into the appropriate wells of a sterile, low binding 96-well polystyrene plate. Direct colony suspension inoculums of the test strains were freshly prepared per CLSI guidelines, and the appropriate test wells were inoculated to achieve a final bacterial cell density of 5×10⁵ CFU/ml. Growth control (no test article) and negative control (no bacterial inoculum) wells were also included. Exposure to light was minimized during all stages of assay preparation. MIC test plates were incubated at 35° C. for 20 hours. Bacterial growth was then determined by measuring the optical density at 600 nm (OD₆₀₀) using a SpectraMax Plus plate-reader spectrophotometer. MIC values were assigned, following assessment of both OD₆₀₀ values and visual inspection of wells, as the lowest test article concentration that resulted in no visible bacterial growth.

Results are shown in Table I below.

TABLE I

| | | | E. coli | A. baumannii | K. pneumoniae | S. aureus |
|---|---|---|---|---|---|---|
| | | A. baumannii | AG100 | ATCC | A6030827 | ATCC |
| Compound | | FabI IC50 | MIC | BAA1605 | MIC | 29213 MIC |
| Number | Compound screened | (nM) | (μg/mL) | MIC (μg/mL) | (μg/mL) | (μg/mL) |
| 11 | (E)-N-methyl-3-(7- | <10 | 8 | 8 | 8 | ≤4 |
| | (methylamino)-8-oxo- | | | | | |
| | 6,7,8,9-tetrahydro-5H- | | | | | |
| | pyrido[2,3-b]azepin-3-yl)- | | | | | |
| | N-((2-methylbenzofuran- | | | | | |
| | 3-yl)methyl)acrylamide | | | | | |
| 13 | (E)-3-[7-(dimethylamino)- | <10 | 16 | ≤4 | 8 | ≤4 |
| | 8-oxo-5,6,7,9- | | | | | |
| | tetrahydropyrido[2,3- | | | | | |
| | b]azepin-3-yl]-N-methyl- | | | | | |
| | N-[(2-methylbenzofuran- | | | | | |
| | 3-yl)methyl]prop-2- | | | | | |
| | enamide | | | | | |
| 15 | (E)-N-methyl-N-[(2- | <10 | 16 | ≤4 | 16 | ≤4 |
| | methylbenzofuran-3- | | | | | |
| | yl)methyl]-3-(8-oxo-7- | | | | | |
| | pyrrolidin-1-yl-5,6,7,9- | | | | | |
| | tetrahydropyrido[2,3- | | | | | |
| | b]azepin-3-yl)prop-2- | | | | | |
| | enamide | | | | | |
| 23 | (E)-3-((2R,3S)-3-amino- | <10 | 8 | ≤4 | 8 | ≤4 |
| | 2-methyl-4-oxo-2,3,4,5- | | | | | |
| | tetrahydro-1H- | | | | | |
| | pyrido[2,3- | | | | | |
| | b][1,4]diazepin-8-yl)-N- | | | | | |
| | methyl-N-((2- | | | | | |
| | methylbenzofuran-3- | | | | | |
| | yl)methyl)acrylamide | | | | | |
| 25 | (S*,E)-N-methyl-N-((2- | <10 | 8 | ≤4 | N/A | ≤4 |
| | methylbenzofuran-3- | | | | | |
| | yl)methyl)-3-(8-oxo-7- | | | | | |
| | (pyrrolidin-1-yl)-6,7,8,9- | | | | | |
| | tetrahydro-5H- | | | | | |
| | pyrido[2,3-b]azepin-3- | | | | | |
| | yl)acrylamide | | | | | |
| 28 | (E)-3-((R*)-7-((R)-3- | <10 | 8 | ≤4 | N/A | ≤4 |
| | hydroxypyrrolidin-1-yl)-8- | | | | | |
| | oxo-6,7,8,9-tetrahydro- | | | | | |
| | 5H-pyrido[2,3-b]azepin- | | | | | |
| | 3-yl)-N-methyl-N-((2- | | | | | |
| | methylbenzofuran-3- | | | | | |
| | yl)methyl)acrylamide | | | | | |
| 39 | (S*,E)-3-(7-amino-7- | <10 | ≤4 | ≤4 | N/A | ≤4 |
| | methyl-8-oxo-6,7,8,9- | | | | | |
| | tetrahydro-5H- | | | | | |
| | pyrido[2,3-b]azepin-3-yl)- | | | | | |
| | N-methyl-N-((2- | | | | | |
| | methylbenzofuran-3- | | | | | |
| | yl)methyl)acrylamide | | | | | |

*stereochemistry arbitrarily assigned

1. A compound of formula (I) 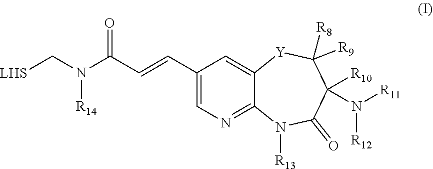
or a pharmaceutically acceptable prodrug, salt and/or solvate thereof, wherein LHS is 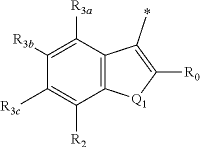
wherein, the asterisk (*) marks the point of attachment;
Y is selected from the group consisting of CH₂, NH, and NR_{d};
Q₁ is selected from the group consisting of O, S, NH and N—C₁₋₄-alkyl;
R₀ is selected from the group consisting of F, CH₂F, CH₃ and Cl, or alternatively R₀ together with R₁₄ form a heterocycle comprising the N to which R₁₄ is attached and having 5 to 8 ring members;
R₂ is selected from the group consisting of H, F, Cl, Br, I, C₁₋₄-alkyl, C₁₋₄-alkylene-OR₅, NR₅R₆, CO—NR₅R₆, C₁₋₄-alkylene-NR₅R₆, C₃₋₆-cycloalkyl, phenyl, and a heterocyclic group having 5 or 6 ring members and 1, 2 or 3 heteroatoms independently selected from N, O and S, wherein said C₁₋₄-alkyl, cycloalkyl, phenyl, or heterocyclic group may optionally be substituted with 1-3 R₇ groups;
R₃ₐ, R_{3b} and R_{3c}, are independently selected from the group consisting of H, F, Cl, Br, I, OH, NH₂, CH₃;
R₅ and R₆ are independently selected from the group consisting of H, C₁₋₄-alkyl, C₃₋₆-cycloalkyl, phenyl, and a heterocyclic group having 5 or 6 ring members and 1, 2 or 3 heteroatoms independently selected from N, O and S, wherein said cycloalkyl, phenyl, or heterocyclic group may optionally be substituted with 1-3 R₇ groups;
R₇ is selected from the group consisting of H, F, I, Br, Cl, O, C₁₋₄-alkyl, CONH₂, OH, NH₂, O—C₁₋₄-alkyl, NH—C₁₋₄-alkyl, N(C₁₋₄-alkyl)₂, C₁₋₄-alkylene-OH, and C₁₋₄-alkylene-NH₂, NO₂, CN, C₂₋₄-alkenyl, C₂₋₄-alkynyl, C₂₋₄-alkynylene-OH, C₂₋₄-alkynylene-NH₂, SO₂CH₃, and O—C₁₋₄-alkylene-OH;
R₁₀ is H or methyl;
R₁₃ is selected from the group consisting of H or R_{d}; R₁₄ is CH₃, or alternatively R₁₄ together with R₀ of LHS form a heterocycle comprising the N to which R₁₄ is attached and having 5 to 8 ring members;
and,
R_{d} is selected from the group consisting of —PO₃Rₑ₂, —CH₂—OPO₃Rₑ₂, wherein Rₑ is selected from the group consisting of H and a cation suitable for forming a pharmaceutically acceptable salt
wherein the compound of formula (I) is further characterized according to one of the following embodiments (A) or (B):
(A)R₈ is H or F, and R₉ is selected from the group consisting of H, F, methyl, ethyl, CN, OH, NH₂ and CH₂—OH; R₁₁ and R₁₂ are independently selected from the group consisting of H, R_{d}, optionally substituted C₁₋₄-alkyl, wherein each of the optionally substituted C₁₋₄-alkyl groups may carry a substituent selected from F, OH, OMe and NH₂, NIMe, NMe₂, or alternatively, R₁₁ and R₁₂ together with the N to which they are attached form a heterocyclic group having 4, 5 or 6 ring members optionally comprising an oxygen atom or nitrogen atom in addition to the nitrogen atom bonded to the bicyclic group and carrying the R₁₁ and R₁₂ groups, wherein said heterocyclic group may carry one or two substituents independently selected from F, methyl, OH and NH₂;
wherein the compound of formula (I), embodiment (A), is none of the following compounds 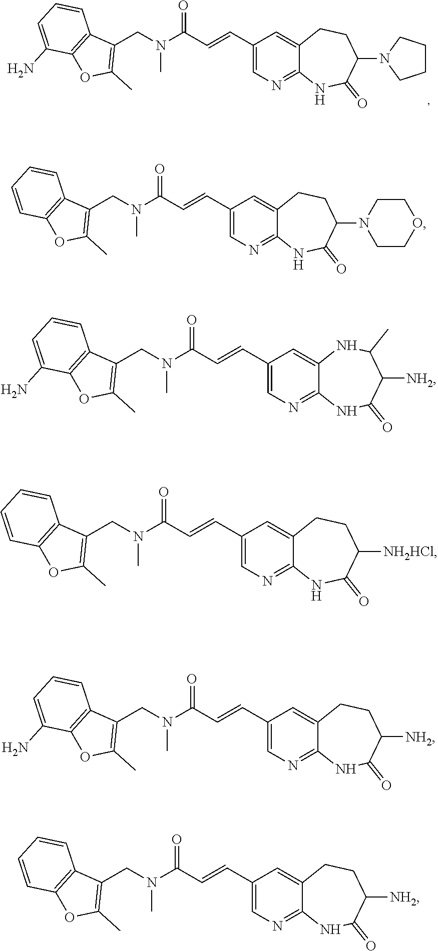
and neither any of the stereoisomers of these compounds such as 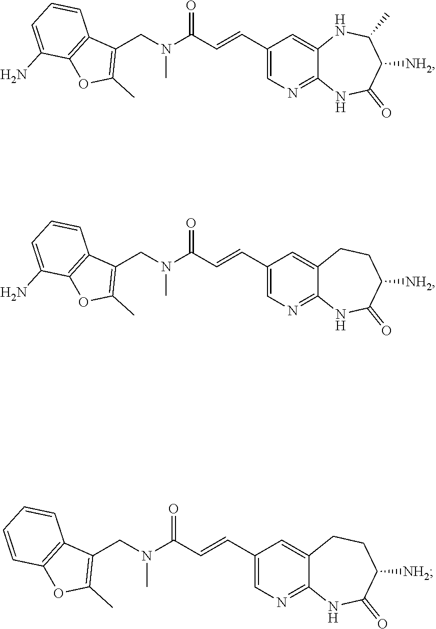 [SMILES: Cc1oc2c(N)cccc2c1CN(C)C(=O)/C=C/c1cnc2c(c1)N[C@H](C)[C@H](N)C(=O)N2 ; Cc1oc2c(N)cccc2c1CN(C)C(=O)/C=C/c1cnc2c(c1)CC[C@H](N)C(=O)N2 ; Cc1oc2ccccc2c1CN(C)C(=O)/C=C/c1cnc2c(c1)CC[C@H](N)C(=O)N2]
(B) the compound of formula (I) is characterized by formula (Ia) 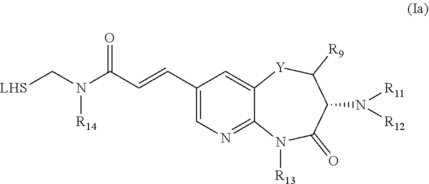
wherein R₉ is a methyl group, hydroxyl group or nitrile group;
R₁₁ and R₁₂ are independently selected from the group consisting of H, R_{d}, C₁₋₄-alkyl, CO—C₁₋₄-alkyl, SO₂(C₁₋₄-alkyl)₁, C₁₋₄-alkyl-F, C₁₋₄-alkylene-OH, and C₁₋₄-alkylene-NH₂, or alternatively, R₁₁ and R₁₂ together with the N to which they are attached form a heterocyclic group having 4 to 9 ring members and 1, 2 or 3 heteroatoms independently selected from N, O and S or R₁₁ and R₁₂ form a heterocyclic spiro group having 7 to 11 ring members and 1, 2 or 3 heteroatoms independently selected from N, O and S, wherein said heterocyclic or heterocyclic spiro group may be substituted with 1-3 R₇ groups,
wherein the compound is diastereomerically pure in relation to the chiral carbon atom carrying R₉ and the chiral carbon atom carrying NR₁₁R₁₂;
wherein the compound of formula (Ia) in embodiment (B) is not

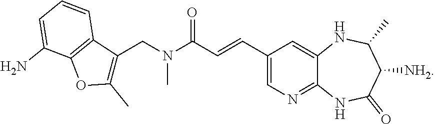 [SMILES: Cc1oc2c(N)cccc2c1CN(C)C(=O)/C=C/c1cnc2c(c1)N[C@H](C)[C@H](N)C(=O)N2]

2. The compound according to claim 1, or a pharmaceutically acceptable prodrug, salt and/or solvate thereof, wherein the compound is a compound of formula (Ia) 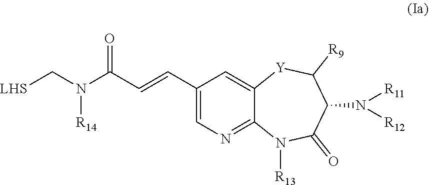
wherein LHS, Y, R₉, R₁₁, R₁₂, R₁₃ and R₁₄ are as specified in claim 1, embodiment (A), and wherein the compound is diastereomerically pure in relation to the chiral carbon atom carrying R₉ and the chiral carbon atom carrying NR₁₁R₁₂.

3. The compound according to claim 1, or a pharmaceutically acceptable prodrug, salt and/or solvate thereof, wherein the chiral carbon atom carrying the R₉ group is beneath the plane of the heterocycle such that the compound is characterized by general formula (Ib)

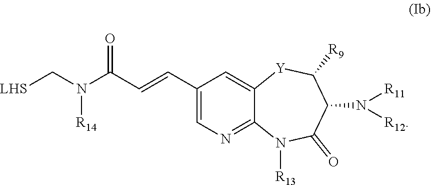

4. The compound according to claim 1, or a pharmaceutically acceptable prodrug, salt and/or solvate thereof, R₁₁ and R₁₂ are selected such that the nitrogen, to which R₁ and R₁₂ are attached, exhibits a pKa in the range of from 6.0 to 8.5.

5. The compound according to claim 1, or a pharmaceutically acceptable prodrug, salt and/or solvate thereof, wherein Q₁ is O, R₀ is CH₃ and/or R₁₄ is CH₃.

6. The compound according to claim 1, or a pharmaceutically acceptable prodrug, salt and/or solvate thereof, wherein R₃ₐ is H, R_{3b} is H, R_{3c}, is H and/or R₂ is selected from the group consisting of H, F, Cl, Br, I and NR₅R₆.

7. The compound according to claim 1, or a pharmaceutically acceptable prodrug, salt and/or solvate thereof, wherein R₉ is selected from H, F, OH, CN, CH₂—OH, or a methyl group.

8. The compound according to claim 1, or a pharmaceutically acceptable prodrug, salt and/or solvate thereof, wherein R₁₁ and R₁₂ are independently selected from H, methyl, ethyl, 2-hydroxyethyl, 2-aminoethyl, or R₁₁ and R₁₂ together with the adjacent nitrogen atom form a heterocycle selected from an azetidine group, a pyrrolidine group, a piperidine group, a morpholine group and a piperazine group, wherein each of these heterocycles may be substituted by a group selected from F, OH, NH₂ and methyl or substituted by two methyl groups, which may be in geminal or different positions.

9. The compound according to claim 1, or a pharmaceutically acceptable prodrug, salt and/or solvate thereof, wherein Y is CH₂.

10. The compound according to claim 1, or a pharmaceutically acceptable prodrug, salt and/or solvate thereof, wherein Y is NH.

11. The compound according to claim 1, or a pharmaceutically acceptable prodrug, salt and/or solvate thereof, wherein
Y is CH₂ or NH;
Q₁ is O;
R₀ is CH₃;
R₁₃ is H or R_{d};
R₁₄ is CH₃;
R₂ is selected from H, F, Cl, Br, I and NR₅R₆;
R₃a is H, R₃b is H and R₃c is H; and
R₁₁ and R₁₂ are independently selected from H, methyl, ethyl, 2-hydroxyethyl, 2-aminoethyl, or R₁₁ and R₁₂ together with the adjacent nitrogen atom form a heterocycle selected from an azetidine group, a pyrrolidine group, a piperidine group, a morpholine group and a piperazine group, wherein each of these heterocycles may be substituted by a group selected from F, OH, NH₂ and methyl or substituted by two methyl groups, which may be in geminal or different positions.

12. The compound according to claim 1, which is selected from the group consisting of 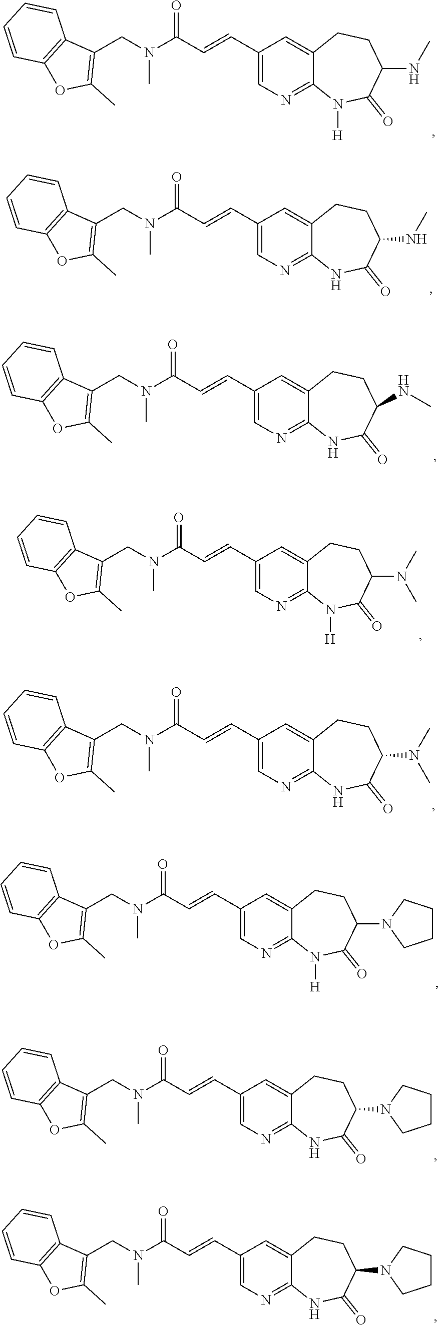 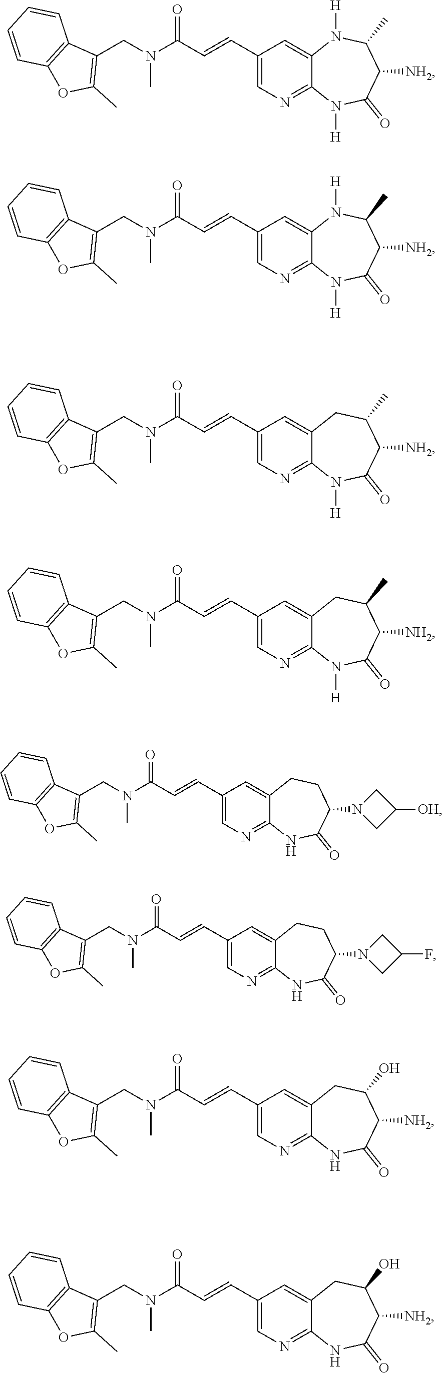 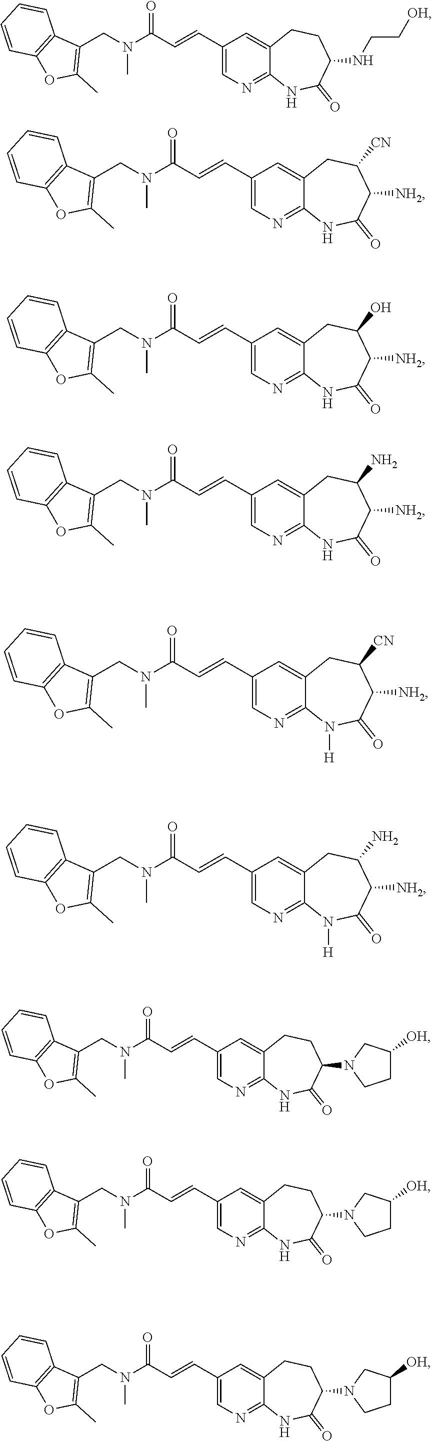 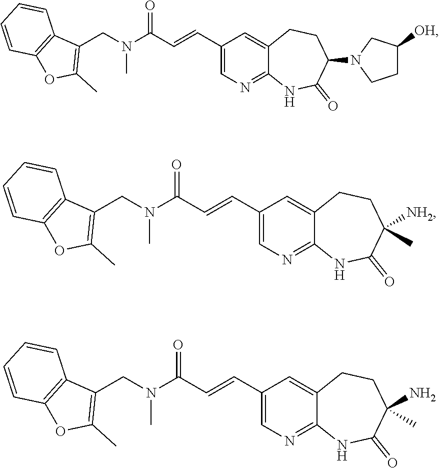 [SMILES: Cc1oc2ccccc2c1CN(C)C(=O)/C=C/c1cnc2c(c1)CC[C@@H](N1CC[C@H](O)C1)C(=O)N2 ; Cc1oc2ccccc2c1CN(C)C(=O)/C=C/c1cnc2c(c1)CC[C@](C)(N)C(=O)N2 ; Cc1oc2ccccc2c1CN(C)C(=O)/C=C/c1cnc2c(c1)CC[C@@](C)(N)C(=O)N2]
and any pharmaceutically acceptable prodrugs, salts and/or solvates thereof.

13. A pharmaceutical composition comprising a compound according to claim 1, or a pharmaceutically acceptable prodrug, salt and/or solvate thereof.

14. A method of treating a bacterial infection comprising administering a compound according to claim 1, or a pharmaceutically acceptable prodrug, salt and/or solvate thereof, or a composition thereof to a subject in need thereof.

15. A method for producing a compound according to claim 1, or a pharmaceutically acceptable prodrug, salt and/or solvate thereof, wherein said method is selected from a first variant that comprises the step of coupling a precursor compound of formula M1 or M1′ 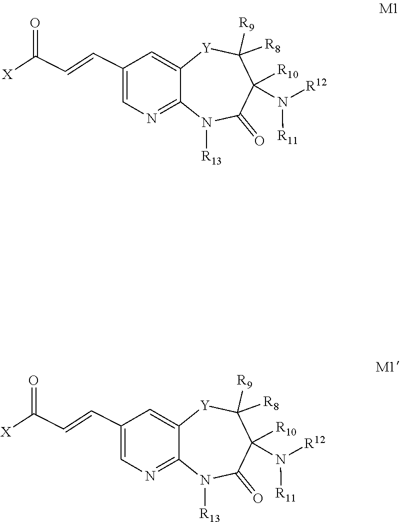
wherein X represents a leaving group, and Pg represents a protective group,
with an amine compound of formula M2b 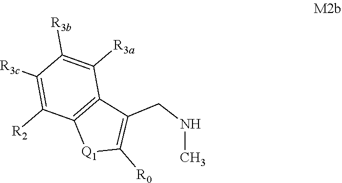
wherein Y, Q₁, and all R groups have the same meanings as specified in claims 1 to 12 and R₁₁ and/or R₁₂ in addition of being defined as specified hereinabove may also comprise a protective group;
and a second variant that comprises the step of coupling a compound of formula M6 or M6′ 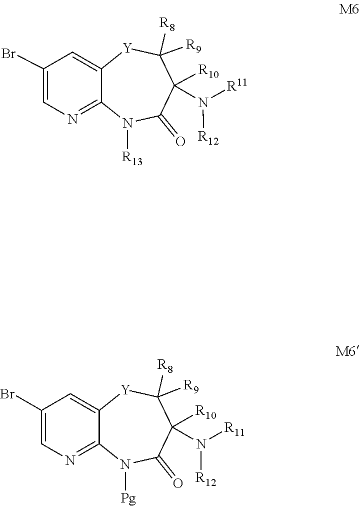
with a compound of formula M7b 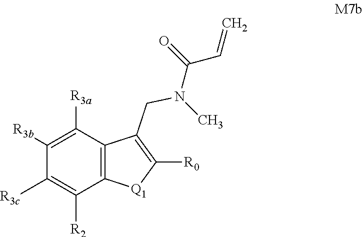
wherein Pg represents a protective group, wherein Y, Q₁ and all R groups have the same meaning as specified in claims 1 to 12, wherein R₁₁ and R₁₂ may be a group as defined in any of claims 1 to 12 or may be such a defined group that also comprises a protective group.

16. The method according to claim 14, wherein the bacterial infection is associated with one or more bacteria selected from the group consisting of: S. aureus, E. coli, Klebsiella pneumoniae and A. baumannii.

17. The method according to claim 14, wherein the bacterial infection is pneumonia.

18. The method according to claim 14, wherein the bacterial infection is nosocomial pneumonia.

19. The method according to claim 15, wherein the leaving group is selected from the group consisting of a hydroxyl group, a tosylate group, a triflate group, a mesylate group, iodide, bromide, chloride, methoxy, and ethoxy.

20. The method according to claim 15, wherein the protective group is selected from the group consisting of a Boc group, PMB group, and DMB group.
